(19) United States
(12) Patent Application Publication
(10) Pub. No.: US 2021/0205365 A1
(43) Pub. Date: Jul. 8, 2021

(54) Chimeric Growth Factor Receptors

(71) Applicant: Instil Bio (UK) Ltd., Manchester (GB)

(72) Inventors: Nicola Kaye Price, Manchester (GB); John Stephen Bridgeman, Manchester (GB)

(21) Appl. No.: 17/124,922

(22) Filed: Dec. 17, 2020

Related U.S. Application Data
(63) Continuation-in-part of application No. PCT/GB2019/051745, filed on Jun. 21, 2019.

(30) Foreign Application Priority Data
Jun. 21, 2018 (GB) 1810181.6

(51) Int. Cl. A61K 35/17; C07K 14/71; C07K 14/725; A61K 38/19; A61K 31/4152

(52) U.S. Cl. CPC A61K 35/17; C07K 14/71; A61K 31/4152; A61K 38/196; C07K 14/7051

(57) ABSTRACT

Adoptive cell therapy involves the transfer of autologous or allogeneic cells to patients in an effort to treat a variety of diseases. In the area of immunotherapy, tumour specific T-cells can be grown ex vivo, or engrafted with tumour specificity via genetic engineering approaches, prior to reinfusion. T-cell infusions require a pre-conditioning treatment, and often a post infusion treatment of IL-2, in an effort to enhance persistence and engraftment. Herein Applicants show that T-cells can be engineered to express a Chimeric recombinant Growth Factor Receptor (CrGFR) which allows the selective survival and/or expansion of T-cells upon administration of a clinically available drug, Eltrombopag.

### RELATED APPLICATIONS AND INCORPORATION BY REFERENCE

This application is a continuation-in-part application of international patent application Serial No. PCT/GB2019/051745 filed Jun. 21, 2019 and published as PCT Publication No. WO2019/243835 on Dec. 26, 2019 and which claims benefit of priority to patent application Serial No. GB 1810181.6 filed Jun. 21, 2018.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing, which has been submitted electronically and is hereby incorporated by reference in its entirety. Said ASCII copy, is named 00011SL.txt and is 570 kb in size.

### FIELD OF THE INVENTION

The present invention relates to T-cells engineered to express a Chimeric recombinant Growth Factor Receptor (CrGFR) which allows the selective survival and/or expansion of T-cells.

### BACKGROUND OF THE INVENTION

Adoptive cell therapy (ACT) using autologous T-cells to mediate cancer regression has shown much promise in early clinical trials. Several general approaches have been taken such as the use of naturally occurring tumour reactive or tumour infiltrating lymphocytes (TILs) expanded ex vivo. Additionally, T-cells may be modified genetically to retarget them towards defined tumour antigens. This can be done via the gene transfer of peptide (p)-major histocompatibility complex (MHC) specific T-cell Receptors (TCRs) or synthetic fusions between tumour specific single chain antibody fragment (scFv) and T-cell signalling domains (e.g. CD3), the latter being termed chimeric antigen receptors (CARs). TIL and TCR transfer has proven particularly good when targeting Melanoma (Rosenberg et al. 2011; Morgan 2006), whereas CAR therapy has shown much promise in the treatment of certain B-cell malignancies (Grupp et al. 2013).

The current general treatment protocol for ACT requires an initial non-myeloablative preconditioning treatment using cyclophosphamide and/or fludarabine which removes most of the circulating lymphocytes in the patients prior to reinfusion of the ex vivo grown cells. This allows space for the new cells to expand and removes potential ‘cytokine sinks’ by which normal cells compete with the newly infused cells for growth and survival signals. Along with the cells patients receive cytokine support via infusions of high doses of interleukin (IL)-2 which helps the new cells engraft and expand.

There are a number of factors which currently limit the technology of T-cell ACT. Current preconditioning therapy described above requires hospital admission and potentially leaves patients in an immunocompromised state. Furthermore, many patients are not in a healthy enough state to be able to withstand the rigours of this treatment regimen. Beyond preconditioning the use of IL-2 as a supportive therapy is associated with severe toxicity and potential intensive care treatment. Indeed, TIL therapy itself, unlike TCR and CAR therapy, has not been associated with any serious on or off target toxicities, with the majority of toxicity events being associated with the accompanying IL-2 infusions.

Methods by which preconditioning and IL-2 supportive treatments can be minimised or reduced will have major benefits in that they will: (i) reduce patient hospitalisation, (ii) increase the proportion of potential patients who could be treated by ACT, (iii) reduce the clinical costs associated with extensive hospital admission, thus again opening up the possibility of ACT to more patients.

### SUMMARY OF THE INVENTION

Thus there is a need for new ACT therapies that minimise the need for preconditioning treatments and/or IL-2 supportive treatments.

The present invention uses cells that express recombinant chimeric growth factor receptors which can be turned on or off by the administration of a ligand for the CrGFR, which may be a clinically validated drug. This permits expansion of target cells in-vivo with minimal toxicity to other cells.

A number of reports have used the idea of growth factor receptor engineering as a means of expanding certain populations of cells or for the development of selection processes for antibody engineering strategies. For example, a number of reports have demonstrated that antibody-TpoR or EpoR fusions could be used to for a number of biotechnology strategies such as single chain antibody selections (Ueda et al. 2000, Kawahara et. Al. 2004), and a number of reports have demonstrated that growth factor receptor fusions can successfully expand the megakaryocyte cell line Ba/F3 and/or haematopoietic stem cells (Jin et al. 2000; Richard et al. 2000; Nagashima et al. 2003; Kawahara et al 2011; Saka et al. 2013).

The thrombopoietin (Tpo) receptor (TpoR; CD110, c-mpl) is normally expressed in cells of the megakaryocyte lineage. In its normal state the TpoR is switched on in response to thrombopoietin, which causes megakaryocyte production of platelets. There is also an active negative feedback loop by which platelet expression of TpoR can be used as a sink to reduce circulating levels of Tpo. Importantly TpoR is not expressed on any other normal tissue or cancer cells (Columbyova 1995).

Recently a report demonstrated that T-cells could be engineered with the wild-type TpoR which could permit controlled survival and expansion of T-cells via administration of Tpo or Eltrombopag (Nishimura et al. 2018). However, there have been no reports of T-cells, or other lymphocytes, being engineered to express chimeric growth factor receptors such as thrombopoietin fusion receptors, and no reports of the use of these cells in ACT.

The present inventors have shown that it is possible to engineer lymphocytes, including T cells and NK cells that comprise a CrGFR that can function as a growth switch. This allows the lymphocytes to be expanded in-vivo by administering the CrGFR ligand to the patient. The inventors have shown that a CrGFR, for example, based on the thrombopoietin (Tpo) receptor (TpoR; CD110, c-mpl), induces proliferation of the engineered lymphocyte following binding of a CrGFR ligand to the receptor. Thus the ligand causes proliferation of cells, or protection from activation-induced cell death, that express the CrGFR but is expected to have low toxicity due to the absence, or low expression, of receptors on other cells in the patient. CrGFRs based on TpoR or other related growth factor receptors would be a valuable tool to augment lymphocyte expansion in vitro and in vivo for adoptive cell therapies.

Thus in a first aspect, the present invention provides a lymphocyte, including a T cell or NK cell, comprising a chimeric recombinant growth factor receptor (CrGFR) comprising:

(i) an extracellular (EC) domain;
(ii) a thrombopoietin transmembrane (TM) domain; and
(iii) a first intracellular (IC) domain; and, optionally, (iv) a second intracellular domain.

The CrGFR is designed such that binding of the receptor ligand to the CrGFR results in receptor activation and growth signalling to the cell to induce proliferation and/or survival.

The ligand may be human thrombopoietin, or a thrombopoietin receptor agonist, e.g. Eltrombopag, Lusotrombopag, Avatrombopag or Romiplastim.

The EC domain may be the human c-mpl EC domain (which binds to human Tpo) or may be one or more of i) a truncated EC domain, ii) a truncated c-mpl EC domain, iii) a selection marker, for example CD34.

The IC domain of the CrGFR may include a JAK binding domain. The IC domain consists of two or more growth factor receptor or other signalling domains where one may be from the list of: human growth hormone receptor, human prolactin receptor or the human thrombopoietin receptor (c-mpl) and additional growth factor or other signalling domains which may be derived from the list of (but not limited to): cytokine receptor signalling domains (e.g. IL2 receptor), Cosignalling domains (e.g. CD40), viral oncogenic proteins (e.g. LMP1), costimulatory domains (e.g. CD28, CD137, CD150 etc) or other mitogenic domains (e.g. Toll like receptors, immunorecptor tyrosine-based activation motifs, CD3 signalling domains etc).

The lymphocyte may be a T cell, including a Tumour Infiltrating Lymphocyte (TIL) a T Regulatory Cell (Treg) or a primary T cell, or an NK cell, or a dendritic cell.

In addition to the CrGFR the lymphocyte, T or NK cell, may include a recombinant T-cell receptor (TCR) or Chimeric Antigen Receptor (CAR).

In a second aspect the invention provides a nucleic acid sequence encoding the CrGFR.

In a third aspect the invention provides a vector which comprises a nucleic acid sequence according to the second aspect and, if present, a TCR and/or CAR nucleic acid sequence.

In a fourth aspect the invention provides a method for making a lymphocyte, or T or NK cell, according to the first aspect of the invention, which comprises the step of introducing a nucleic acid encoding the CrGFR, or vector, into the lymphocyte.

In a fifth aspect the invention provides a pharmaceutical composition which comprises a vector according to the third aspect, or lymphocyte (including a T or NK cell) according to the first aspect, together with a pharmaceutically acceptable carrier, diluent or excipient.

In a sixth aspect the invention provides a method of in-vivo cell expansion comprising administering the lymphocytes, or T or NK cells, of the first aspect, or pharmaceutical composition of the fifth aspect to a subject. The cells may be expanded in-vivo by administering thrombopoietin, or a thrombopoietin agonist such as Eltrombopag, to a subject.

In a seventh aspect the invention provides a lymphocyte, including a T or NK cell, according to the first aspect, or vector according to the third aspect, for use in adoptive cell therapy.

In an eighth aspect the invention provides a lymphocyte, including a T or NK cell, according to the first aspect, or vector according to the third aspect, for use in a method of treating cancer.

In a ninth aspect the invention provides the use of a lymphocyte according to the first aspect, or the use of the vector according to the third aspect in the manufacture of a medicament for treating cancer.

In a tenth aspect the invention provides Eltrombopag or Tpo for use in adoptive cell therapy.

In an eleventh aspect the invention provides Eltrombopag or Tpo for use in the in-vivo expansion of lymphocytes, including T or NK cells.

In a twelfth aspect the invention provides a lymphocyte of the first aspect for use in combination with thrombopoietin or a thrombopoietin receptor agonist, for example Eltrombopag, in the treatment of a cancer.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1—Schematic representation of Chimeric recombinant Growth Factor Receptors containing growth factor domains. These receptors consist of the TpoR extracellular domain and transmembrane domain which spans the plasma membrane. The intracellular domain consists of the TpoR cytoplasmic domain fused to one or more additional domains which augment the overall activity of the receptor and may be derived from a selection of a growth factor domain, cosignalling domain or costimulatory domain as detailed in the figure legend. Δ60=TpoR with 60 amino acid C-terminus deletion, IL2rβcyt=cytoplasmic domain of IL2 receptor beta chain, SLAM=SLAM/CD150, TIAF1=TGFβ1 induced anti-apoptotic factor 1, TLR1=Toll-like receptor 1, CD40=CD40/TNFRSF5, IL2rγ=IL-2 receptor common gamma chain, ITAM1=Immunoreceptor tyrosine based activation motif from CD3, LMP1=Epstein Barr Virus Latent membrane protein 1.

FIG. 2—Schematic representation of Chimeric recombinant Growth Factor Receptors containing costimulatory domains. These receptors consist of the TpoR extracellular domain and transmembrane domain which spans the plasma membrane. The intracellular domain consists of a costimulatory domain obtained from a defined costimulatory receptor such as, but not limited to, CD28 or CD137.

FIG. 3—Schematic representation of the gene organisation of the lentiviral transgene. The TpoR transgene was codon optimised and cloned downstream of the EF1α promoter by way of an XbaI and NheI restriction digest pair in the pSF. Lenti Lentiviral vector.

FIG. 4—Flow analysis of non-transduced, wildtype (WT) and variant Chimeric recombinant Growth Factor Receptors in Jurkat E6.1 cells. Jurkat E6.1 T-cells were transduced with lentiviral particles carrying the indicated transgenes. Expression was assessed 72 h post infection using anti-CD110-PE antibodies.

FIGS. 5A-5B—Analysis of Chimeric recombinant Growth Factor Receptor activity in Ba/F3 cells. The cytokine dependent murine B-cell line Ba/F3 was transduced with the indicated CrGFRs and Incubated with either IL-3 or Eltrombopag for 10 days. Expression of CrGFR was assessed by flow cytometry at the indicated time points using CD110 antibodies.

FIGS. 6A-6B—Analysis of Eltrombopag and IL-2 on primary human T-cells from Donor 1. Primary human T-cells from donor 1 were transduced with the WT TpoR or variant CrGFR and incubated in the presence of IL2 or Eltrombopag. Cells were removed at time points up to 21 days and the proportion of cells expressing the receptor assessed using PE conjugated anti-CD110 antibodies and a MACSQuant analyser.

FIGS. 7A-7B—Analysis of Eltrombopag and IL-2 on primary human T-cells from Donor 2. Primary human T-cells from donor 2 were transduced with the WT TpoR or variant CrGFR and incubated in the presence of IL2 or Eltrombopag. Cells were removed at time points up to 21 days and the proportion of cells expressing the receptor assessed using PE conjugated anti-CD110 antibodies and a MACSQuant analyser.

FIGS. 8A-8B—Analysis of Eltrombopag and IL-2 on primary human T-cells from Donor 3. Primary human T-cells from donor 3 were transduced with the WT TpoR or variant CrGFR and incubated in the presence of IL2 or Eltrombopag. Cells were removed at time points up to 21 days and the proportion of cells expressing the receptor assessed using PE conjugated anti-CD110 antibodies and a MACSQuant analyser.

FIGS. 9A-9B—Selection of optimal CrGFRs for next round of analysis. Flow cytometry plots showing expression of CrGFRs in ×3 donor primary human T-cells after 21 days incubation in Eltrombopag. The receptors TpoR.CD40, TpoR.IL2rγ, TpoR.ITAM1, TpoR.A60, TpoR.LMP1-cyto and TpoR.TpoR-cyto.LMP1-cyto were chosen for future comparison with the wt TpoR.

FIG. 10—Analysis of Eltrombopag and IL-2 on CrGFR sorted primary human T-cells from Donor 4. Primary human T-cells from donor 4 were transduced with the WT TpoR or variant CrGFR, and enriched for expression by Miltenyi MACS technology selected for and incubated in the presence of IL2 or Eltrombopag. Cells were removed at time points up to 7 days and the number of cells expressing the receptor assessed using PE conjugated anti-CD110 antibodies, DRAQ7 viability dye and a MACSQuant analyser.

FIG. 11—Analysis of Eltrombopag and IL-2 on CrGFR sorted primary human T-cells from Donor 5. Primary human T-cells from donor 5 were transduced with the WT TpoR or variant CrGFR, and enriched for expression by Miltenyi MACS technology selected for and incubated in the presence of IL2 or Eltrombopag. Cells were removed at time points up to 7 days and the number of cells expressing the receptor assessed using PE conjugated anti-CD110 antibodies, DRAQ7 viability dye and a MACSQuant analyser.

FIG. 12—Analysis of Eltrombopag and IL-2 on CrGFR sorted primary human T-cells from Donor 6. Primary human T-cells from donor 6 were transduced with the WT TpoR or variant CrGFR, and enriched for expression by Miltenyi MACS technology selected for and incubated in the presence of IL2 or Eltrombopag. Cells were removed at time points up to 7 days and the number of cells expressing the receptor assessed using PE conjugated anti-CD110 antibodies, DRAQ7 viability dye and a MACSQuant analyser.

FIG. 13—Analysis of Chimeric recombinant Growth Factor Receptors in TIL042. Tumour Infiltrating Lymphocytes from TIL042 were transduced with the WT TpoR or indicated variant CrGFR and incubated in the presence of patient matched tumour lines with the addition of IL2, Eltrombopag, IL-2+Eltrombopag, or no growth factors. Cells were analysed and counted at days 4 and 7 and the number of cells expressing the receptor assessed using PE conjugated anti-CD110 antibodies, DRAQ7 viability dye and a MACSQuant analyser. Graphs show counts between days 4 and 7 when recovery of TIL occurs after an initial contraction in numbers driven by tumour regulatory factors and/or activation induced cell death.

FIG. 14—Analysis of Chimeric recombinant Growth Factor Receptors in Ovarian TIL. Tumour Infiltrating Lymphocytes from ×3 ovarian TIL were transduced with the WT TpoR or indicated variant CrGFR and incubated in the presence of patient matched tumour cells with either Eltrombopag or no growth factors. Cells were analysed and counted at days 4 and 7 and the number of cells expressing the receptor assessed using PE conjugated anti-CD110 antibodies, DRAQ7 viability dye and a MACSQuant analyser. Graphs show counts between days 4 and 7 when recovery of TIL occurs after an initial contraction in numbers driven by tumour regulatory factors and/or activation induced cell death.

FIG. 15—Induction of pSTAT by chimeric recombinant growth factor receptors. Primary human T-cells were isolated and transduced with the indicated CrGFR. Cells were enriched for CrGFR expression using Miltenyi MACS technology and expanded via polyclonal stimulation. The enriched cells were stimulated for 4 h with either media alone (RPMI), IL2, IL12, Tpo or Eltrombopag (Elt) before methanol fixation and intracellular staining with antibodies towards phospho-STAT5.

### DETAILED DESCRIPTION OF THE INVENTION

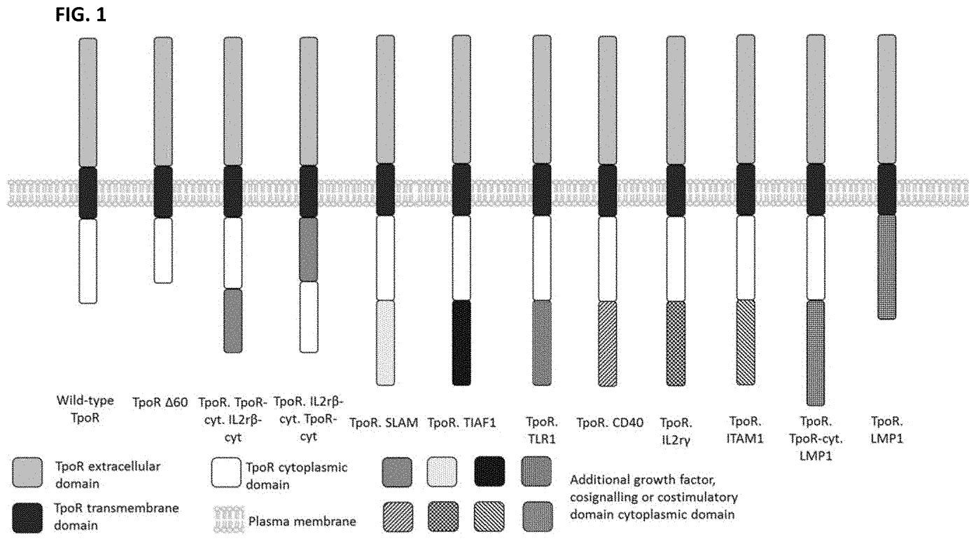

Provided herein are recombinant growth factor receptors (CrGFR) comprising: (i) an extracellular (EC) domain; (ii) a thrombopoietin transmembrane (TM) domain; and (iii) a chimeric growth factor receptor intracellular (IC) domain. In a simple form the CrGFR may contain the full length human Tpo receptor (as provided in FIG. 1 herein) or derivative or variant thereof that maintains signalling and cell proliferation in response to ligand binding (for example this may include a truncated thrombopoietin signalling domain which has been shown to maintain signalling capacity). The CrGFR may be of modular form with the EC, TM and IC domains derived from different receptors. However, the CrGFR must maintain its ability to transmit a growth signal to the cell upon ligand binding. The CrGFR may be activated and transmit a growth signal to the cell upon ligand binding to the TM domain. The signalling domain may contain one or more additional signalling domains.

Suitable CrGFRs may be selected based on GFRs with limited expression on normal human tissue, for example, GFRs that are expressed on only a small cell population or confined to a specific cell type, for example, c-kit. Alternatively, the native ligand binding domain of the growth factor receptor may be removed and e.g. replaced with a marker or other EC domain.

The CrGFR may comprise an EC domain without growth factor binding function (for example a truncated form of the TpoR EC domain) and/or a marker, for example CD34), and the TM and IC domains from TpoR. Growth of cells carrying this type of receptor may then be stimulated by Eltrombopag binding to the TM domain.

The CrGFR may be expressed alone under the control of a promoter in a therapeutic population of cells that have therapeutic activity, for example, Tumour Infiltrating Lymphocytes (TILs).

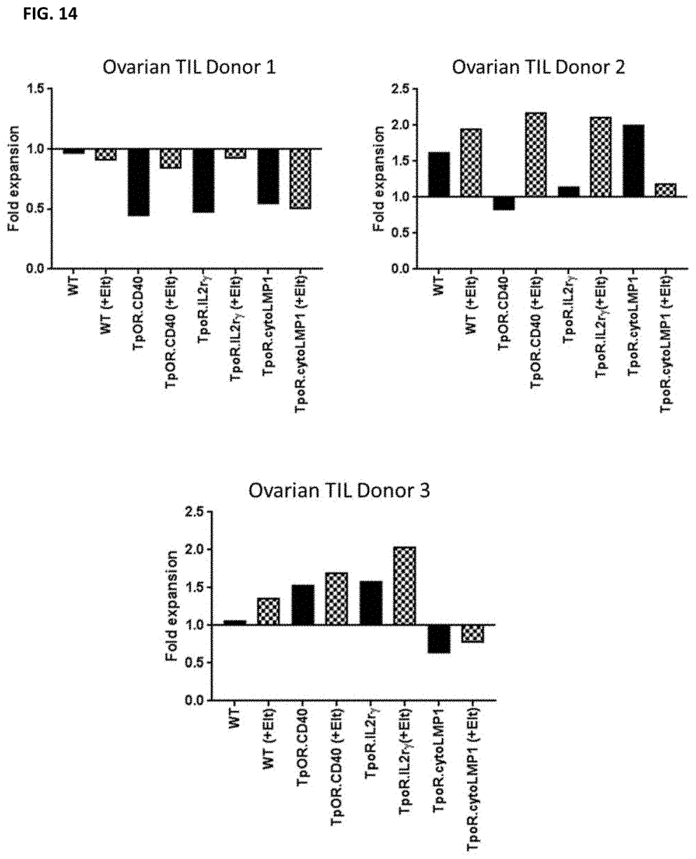

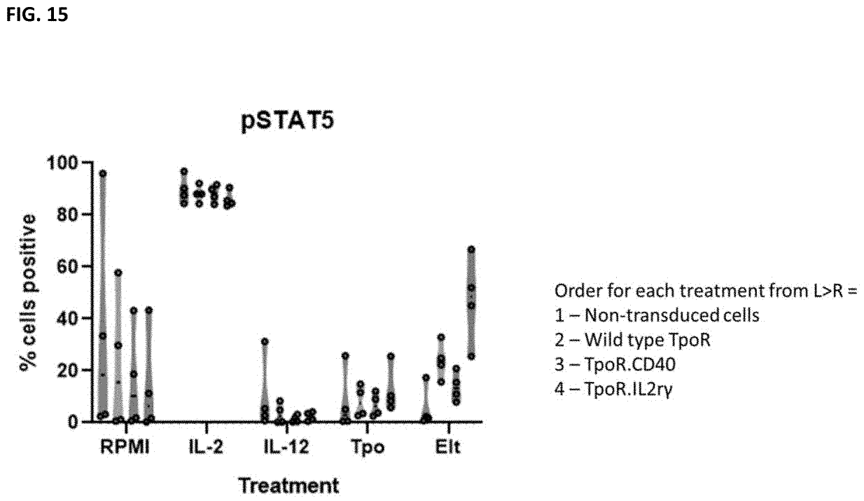

Alternatively, the CrGFR may be expressed along with a therapeutic transgene such as a Chimeric Antigen Receptor (CAR) and/or T-cell Receptor (TCR), for example as described in FIG. 14. Suitable TCRs and CARs are well known in the literature, for example HLA-A*02-NYESO-1 specific TCRs (Rapoport et al. Nat Med 2015) or anti-CD19scFv.CD3ζ fusion CARs (Kochenderfer et al. J Clin Oncol 2015) which have been successfully used to treat Myeloma or B-cell malignancies respectively. The CrGFRs described herein may be expressed with any known CAR or TCR thus providing the cell with a regulatable growth switch to allow cell expansion/survival in-vitro or in-vivo, and a conventional activation mechanism in the form of the TCR or CAR for anti-cancer activity. Thus the invention provides a cell for use in adoptive cell therapy comprising a CrGFR as described herein and a TCR and/or CAR that specifically binds to a tumour associated antigen.

The CrGFR may have the TM domain and first IC domain of the human Tpo receptor and a wildtype or truncated Tpo receptor EC domain (without native ligand binding function).

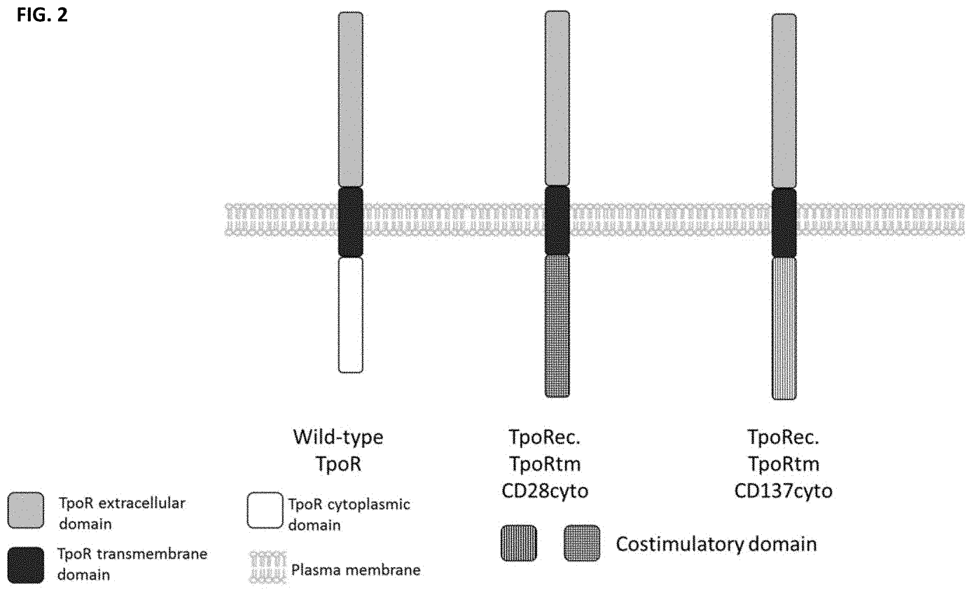

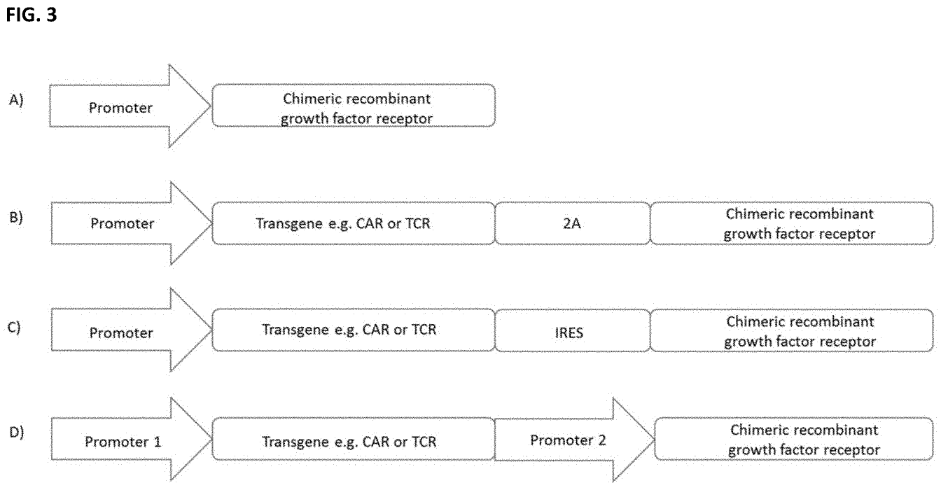

Particular embodiments of the CrGFR include those shown in FIGS. 1 and 2.

In some embodiments the growth factor receptor (CrGFR) is constructed such that the CrGFR is based on the TpoR receptor with at least the TM region and IC region (see SEQ ID NO: 1 which shows the TpoR TM domain and 514-635 and TpoR cytoplasmic domain) being retained and with an additional (second) IC domain being added to the construct to enhance signalling in response to Tpo or Tpo agonist binding. Thus in some embodiments the CrGFR comprises: (i) an TpoR extracellular (EC) domain, or a truncated TpoR EC domain; (ii) a thrombopoietin transmembrane (TM) domain; and (iii) a first intracellular (IC) domain comprising a human thrombopoietin IC domain (or a truncated version thereof, e.g delta 60); and (iv) a second intracellular domain, wherein the second intracellular domain is selected from an IC domain from a costimulatory receptor, a cytokine receptor, a cosignalling receptor, or human thrombopoietin receptor (c-mpl). For example, the second IC domain may the IC domain from CD40, IL2R 4243, IL2Rγ), ITAM1 or LMP1.

In some embodiments the crGFR comprises i) an EC domain; and the TM and IC domains shown in SEQ ID NOS: 3, 4, 5, 6, 7, 8, 9, 10, 11, 12, 13, or 14, or variants thereof having at least 80%, 85%, 90% 95% 97% or 99% sequence identity. Suitable EC domains include those described herein, for example a truncated TpoR EC domain. These receptors retain their ability to bind human thrombopoietin or a thrombopoietin receptor agonist.

In other embodiments the IC domain of wt Tpo is replaced with an IC domain from a suitable receptor, for example LMP1, IL2R, CD28 or CD137; examples of such constructs are shown in FIG. 1 as and “TpoR. LMP1” “TpoR. IL2rβ-cyt.TpoR-cyt” and FIG. 2 “TpoRec.TpoRtm CD28cyto” and “TpoRec.TpoRtm CD137cyto”.

The EC domain may be the EC domain from TpoR (SEQ ID NO: 1) or derivative or variant thereof that maintains signalling and cell proliferation in response to ligand binding to the receptor.

The EC domain may not be required for CrGFR signalling for example if TM domain is used that can cause receptor activation upon ligand binding e.g. the TpoR TM domain. The EC domain may then be a truncated or mutated native domain (e.g. without ligand binding function), for example, a truncated TpoR EC domain. The native EC domain may be replaced by a marker such as truncated CD34 for selection and/or in vivo monitoring.

The TM domain (shown in FIG. 1) from the Tpo receptor (TpoR) may be used, including a derivative or variant thereof that maintains signalling and cell proliferation in response to ligand binding to the receptor. This may be useful because TpoR is known to have limited expression in normal human tissues and it is also known to bind to Eltrombopag Lusutrombopag and Avatrombopag, thus a CrGFR comprising a TM domain from the Tpo receptor can a be activated by exposing the cells in-vitro or in-vivo to a clinically validated compound with a known toxicity profile.

The growth factor receptor intracellular (IC) domain (shown in SEQ ID NO: 1) from the Tpo receptor may be used including a derivative or variant thereof that maintains signalling and cell proliferation in response to ligand binding to the receptor (e.g. a truncated TpoR signalling domain such as that shown in SEQ ID NO: 2). This may be combined with the TM domain from the Tpo receptor to achieve good levels of cell proliferation in response to ligand binding.

Other IC domains that are growth factor receptor like may be suitable for use in constructing the CrGFRs of the present invention, as these receptors are known to activate the same cell signalling pathways as the Tpo receptor. For example, the IC domains from G-CSF, GM-CSF, prolactin or human growth hormone may be used to construct CrGFRs when combined with the TpoR TM domain. The ability of a CrGFR comprising these IC domains to induce cell proliferation in response to a receptor agonist, for example, Eltrombopag, may then be determined using the methods described in the Examples herein. The TpoR IC domain may be truncated by up to 79 amino acids at the C-terminus. Truncations above this have been shown to completely knock out TpoR activity (Gurney et al. PNAS 1995).

Additionally, the IC domain may also comprise a second domain derived from one of the following (but not limited to): cytokine receptor signalling domains (e.g. IL2 receptor), Cosignalling domains (e.g. CD40), viral oncogenic proteins (e.g. LMP1), costimulatory domains (e.g. CD28, CD137, CD150 etc) or other mitogenic domains (e.g. Toll like receptors, immunoreceptor tyrosine-based activation motifs, CD3 signalling domains etc).

Cytokine receptors are a broad group of receptors expressed on a multitude of cell types and are involved in sensing extracellular environmental cues by binding to soluble cytokines. This binding event elicits a signalling cascade via JAK/STAT signalling resulting in upregulation of genes involved in survival and expansion. Such receptors include the IL-2 receptor, IL-4 receptor and Thrombopoietin receptor (Liongue et al. 2016). Costimulatory receptors are proteins involved in enhancing the activity of T-cells when the cell receives a primary signal through the T-cell receptor. This is based on the concept of Signal 1 and Signal 2, whereby Signal 1 is delivered through engagement of T-cell receptor with peptide-MHC, and signal 2 is delivered through engagement of costimulatory receptors on the T-cell with costimulatory ligands on the target cells (e.g. dendritic cell). The signal 2 delivered through the costimulatory domain provides crucial survival signals for the T-cell. Common costimulatory receptors include CD28, CD137 and CD150 (Leitner et al. 2010). The term cosignalling defines groups of cell membrane proteins which provide similar supportive signals to those described for costimulatory receptors but under certain circumstances may not normally be considered co-stimulatory as they may not be expressed on T-cells, such receptors include CD40 which is normally expressed in antigen presenting cells where it enhances survival upon engagement of CD40-ligand expressed on T-cells (He et al. 2012; Kumar et al. 2018).

This second IC domain may be fused directly, or via a linker domain, to the C-terminus of the first IC domain (e.g TpoR IC domain which is disposed next to the transmembrane Tpo domain). Thus the chimeric growth factor receptor may comprise a TpoR transmembrane domain and a TpoR IC domain (first IC domain) and a second IC domain which may be from TpoR, or may be a cytokine receptor signalling domain, Cosignalling domain, viral oncogenic proteins (e.g. LMP1) or costimulatory domains such as those discussed in the preceding paragraph.

Additionally the costimulatory, coinhibitory or cosignalling domain may be fused directly to the TpoR transmembrane domain to create receptors such as those shown in FIG. 2 and SEQ ID NO: 13 and 14. These receptors may comprise a further (second) IC domain, such as a TpoR domain.

The cells used in the present invention may be any lymphocyte that is useful in adoptive cell therapy, such as a T-cell or a natural killer (NK) cell, an NKT cell, a gamma/delta T-cell or T regulatory cell. The cells may be allogenic or autologous.

T cells or T lymphocytes are a type of lymphocyte that have a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Cytotoxic T cells (TC cells, or CTLs) destroy virally infected cells and tumor cells, and are also implicated in transplant rejection. CTLs express the CD8 molecule at their surface. These cells recognize their targets by binding to antigen associated with WIC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3.

Adaptive Treg cells (also known as Tr1 cells or Th3 cells) may originate during a normal immune response.

Natural Killer Cells (or NK cells) are a type of cytolytic cell which form part of the innate immune system. NK cells provide rapid responses to innate signals from virally infected cells in an MHC independent manner.

NK cells (belonging to the group of innate lymphoid cells) are defined as large granular lymphocytes (LGL) and constitute the third kind of cells differentiated from the common lymphoid progenitor generating B and T lymphocytes.

An aspect of the invention provides a nucleic acid sequence of the invention, encoding any of the CrGFRs, polypeptides, or proteins described herein (including functional portions and functional variants thereof).

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed, e.g. codon optimisation.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

The nucleic acid sequence may encode the protein sequences shown in SEQ ID NOS: 3 to 14 or variants thereof, including a nucleic acid sequence encoding or comprising a truncated form of the Tpo receptor such as that shown in SEQ ID NO: 2.

The nucleotide sequence may comprise the nucleotide sequence of TpoR shown in SEQ ID NOS: 17 to 28, or variants thereof.

The invention also provides a nucleic acid sequence which comprises a nucleic acid sequence encoding a CrGFR and a further nucleic acid sequence encoding a T-cell receptor (TCR) and/or chimeric antigen receptor (CAR).

The nucleic acid sequences may be joined by a sequence allowing co-expression of the two or more nucleic acid sequences. For example, the construct may comprise an internal promoter, an internal ribosome entry sequence (IRES) sequence or a sequence encoding a cleavage site. The cleavage site may be self-cleaving, such that when the polypeptide is produced, it is immediately cleaved into the discrete proteins without the need for any external cleavage activity.

Various self-cleaving sites are known, including the Foot-and-Mouth disease virus (FMDV) and the 2a self-cleaving peptide.

The co-expressing sequence may be an internal ribosome entry sequence (IRES). The co-expressing sequence may be an internal promoter.

In an aspect, the present invention provides a vector which comprises a nucleic acid sequence or nucleic acid construct of the invention.

Such a vector may be used to introduce the nucleic acid sequence(s) or nucleic acid construct(s) into a host cell so that it expresses one or more CrGFR(s) according to the first aspect of the invention and, optionally, one or more other proteins of interest (POI), for example a TCR or a CAR.

The vector may, for example, be a plasmid or a viral vector, such as a retroviral vector or a lentiviral vector, or a transposon based vector or synthetic mRNA. Vectors derived from retroviruses, such as the lentivirus, are suitable tools to achieve long-term gene transfer since they allow long-term, stable integration of a transgene or transgenes and its propagation in daughter cells.

The vector may be capable of transfecting or transducing a lymphocyte including a T cell or an NK cell.

The present invention also provides vectors in which a nucleic acid of the present invention is inserted.

The expression of natural or synthetic nucleic acids encoding a CrGFR, and optionally a TCR or CAR is typically achieved by operably linking a nucleic acid encoding the CrGFR and TCR/CAR polypeptide or portions thereof to one or more promoters, and incorporating the construct into an expression vector. The vectors can be suitable for replication and integration in eukaryotic cells. Typical cloning vectors contain transcription and translation terminators, initiation sequences, and promoters useful for regulation of the expression of the desired nucleic acid sequence.

Viral vector technology is well known in the art and is described, for example, in Sambrook et al. (2001, Molecular Cloning: A Laboratory Manual, Cold Spring Harbor Laboratory, New York), and in other virology and molecular biology manuals, see also, WO 01/96584; WO 01/29058; and U.S. Pat. No. 6,326,193).

In some embodiments, the nucleic acid constructs are as shown in the figures herein. In some embodiments the nucleic acids are multicystronic constructs that permit the expression of multiple transgenes (e.g., CrGFR and a TCR and/or CAR etc.) under the control of a single promoter. In some embodiments, the transgenes (e.g., CrGFR and a TCR and/or CAR etc.) are separated by a self-cleaving 2A peptide. Examples of 2A peptides useful in the nucleic acid constructs of the invention include F2A, P2A, T2A and E2A. In other embodiments of the invention, the nucleic acid construct of the invention is a multicystronic construct comprising two promoters; one promoter driving the expression of CrGFR and the other promoter driving the expression of the TCR or CAR. In some embodiments, the dual promoter constructs of the invention are uni-directional. In other embodiments, the dual promoter constructs of the invention are bi-directional.

In order to assess the expression of the CrGFR polypeptide or portions thereof, the expression vector to be introduced into a cell can also contain either a selectable marker gene or a reporter gene or both to facilitate identification and selection of expressing cells from the population of cells sought to be transfected or transduced through viral vectors. The CrGFR polypeptide may incorporate a marker, such as CD34, as part of the EC domain.

The present invention also relates to a pharmaceutical composition containing a vector or a CrGFR expressing cell of the invention together with a pharmaceutically acceptable carrier, diluent or excipient, and optionally one or more further pharmaceutically active polypeptides and/or compounds. Such a formulation may, for example, be in a form suitable for intravenous infusion.

Cells, including T and NK cells, expressing CrGFRs for use in the methods of the present may either be created ex vivo either from a patient's own peripheral blood (autologous), or in the setting of a haematopoietic stem cell transplant from donor peripheral blood or peripheral blood from an unconnected donor (allogenic). Alternatively, T-cells or NK cells may be derived from ex-vivo differentiation of inducible progenitor cells or embryonic progenitor cells to T-cells or NK cells. In these instances, T-cells expressing a CrGFR and, optionally, a CAR and/or TCR, are generated by introducing DNA or RNA coding for the CrGFR and, optionally, a CAR and/or TCR, by one of many means including transduction with a viral vector, transfection with DNA or RNA.

T or NK cells expressing a CrGFR of the present invention and, optionally, expressing a TCR and/or CAR may be used for the treatment of haemotological cancers or solid tumours.

A method for the treatment of disease relates to the therapeutic use of a vector or cell, including a T or NK cell, of the invention. In this respect, the vector, or T or NK cell may be administered to a subject having an existing disease or condition in order to lessen, reduce or improve at least one symptom associated with the disease and/or to slow down, reduce or block the progression of the disease. The method of the invention may cause or promote T-cell mediated killing of cancer cells.

The vector, or T or NK cell according to the present invention may be administered to a patient with one or more additional therapeutic agents. The one or more additional therapeutic agents can be coadministered to the patient. By “coadministering” is meant administering one or more additional therapeutic agents and the vector, or T or NK cell of the present invention sufficiently close in time such that the vector, or T or NK cell can enhance the effect of one or more additional therapeutic agents, or vice versa. In this regard, the vectors or cells can be administered first and the one or more additional therapeutic agents can be administered second, or vice versa. Alternatively, the vectors or cells and the one or more additional therapeutic agents can be administered simultaneously. Suitable therapeutic agents that may be co-administered with the vectors or cells of the present invention include any growth factor receptor agonist that activates the CrGFR, for example, Eltrombopag (rINN, codenamed SB-497115-GR) Lusutrombopag and Avatrombopag or Romiplostim.

Eltrombopag may be particularly useful in the methods of the invention as its toxicity profile is known. In preclinical studies, the compound was shown to interact selectively with the thrombopoietin receptor, leading to activation of the JAK-STAT signalling pathway and increased proliferation and differentiation of megakaryocytes. Animal studies confirmed that administration could increase platelet counts. In 73 healthy volunteers, higher doses of Eltrombopag caused larger increases in the number of circulating platelets without tolerability problems, see, for example, Jenkins J M, Williams D, Deng Y, Uhl J, Kitchen V, Collins D, Erickson-Miller C L (June 2007). “Phase 1 clinical study of eltrombopag, an oral, nonpeptide thrombopoietin receptor agonist”. Blood 109 (11): 4739-41. Thus in the methods of the invention suitable dosages of Eltrombopag may be determined based on previously published clinical studies and the in-vitro assays described herein.

Another agent that may be useful is IL-2, as this is currently used in existing cell therapies to boost the activity of administered cells. However, as stated earlier, IL-2 treatment is associated with toxicity and tolerability issues. Thus it is an aim of present invention to stimulate cell proliferation using an agonist that binds to the CrGFR and, therefore, reduce the amount of IL-2 that must be administered (e.g. to levels that are less toxic) or even eliminate the need for IL-2 administration.

For purposes of the inventive methods, wherein cells are administered to the patient, the cells can be cells that are allogeneic or autologous to the patient.

Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

Certain aspects and embodiments of the invention will now be illustrated by way of example and with reference to the figures described above and tables described below.

### EXAMPLES

### Example 1—Production and Evaluation of T-Cells Expressing CrGFR

### Materials and Methods

### Plasmids

The pSF.Lenti.EF1α plasmid was generated by Oxford Genetics by replacing the existing CMV promoter in pSF.Lenti.CMV.PGK.puro with the elongation factor (EF)1α promoter to generate pSF.Lenti.EF1α.PGK.puro. The PGK.Puro segment was then removed by and TpoR constructs cloned in via an XbaI/NheI digestion with the NheI site downstream of the puromycin resistance gene. The packaging plasmids pVSVg, pCgpV and pRSV.Rev (ViraSafe Lentiviral packaging system—Pantropic) were obtained from Cell Biolabs (VPK-206).

### Reagents

The following reagents were sourced from the following manufacturers:—

Abcam—DRAQ7 (AB 109202-1 ml)

Miltenyi Biotec—anti-Melanoma (MCSP)-PE (130-099-413); anti-CD34-APC (130-090-954), anti-CD45-FITC (130-080-202), anti-CD71-APC (130-099-239), anti-CD110-PE

BD Biosciences—anti-CD34-PE (555822);

E-Biosciences—Fixable Viability dye eFlor 450 (65-0863-18), Fixable Viability dye eFlor 780 (65-0865-18),

### Cell Lines

The Jurkat E6.1 cell line and Ba/F3 cell line were cultured in RPMI supplemented with % FCS (F9665-500 ml: Sigma), 1% 1M HEPES (H0887-100 ml) and 1% Penicillin/streptomycin (P0781-100 ml) (T-cell media: TCM). The cell line 293T and was routinely cultured in DMEM supplemented with 10% FCS and 1% Penicillin/streptomycin (P0781-100 ml) (D10).

### T-Cell Isolation

T-cells were isolated from PBMC from buffy coats. In brief buffy coats were obtained from NHSBT, and PBMC isolated by Ficoll-mediated density centrifugation. Untouched T-cells were isolated using paramagnetic beads (see below). T-cells were cultured in RPMI supplemented with 10% FCS (F9665-500 ml: Sigma), 1% 1M HEPES (H0887-100 ml) and 1% Penicillin/streptomycin (P0781-100 ml) (T-cell media: TCM).

### Lentivirus Production

6×10⁶ 293 T cells were plated in 10 ml D10 the day prior to transfection in a poly-d-lysine coated T75 flask (Greiner). On the day of transfection 0.025 M HEPES buffered serum-free DMEM (pH 7.1) and 0.025 M HEPES buffered D10 (pH 7.9) were prepared. 1.5 ml transfection mixes were prepared per flask using 10 μg lentiviral transfer plasmid (pSF.Lenti) and 10 μg each of pVSVg, pCgpV and pRSV.Rev and CaCl2 to a final concentration of 0.05 M in pH7.1 media. Transfection complexes were allowed to form for 30 min before being added dropwise to the flasks containing 6 ml pH7.9 media. 24 h later the media was exchanged for 10 ml fresh D10. 24 and 48 h later the media was harvested, combined and concentrated using Lenti-X concentrator (Clontech-Takara: 631232). Concentrated lentiviral particles were resuspended at 10× the original supernanat volume and stored at −80° C. until use.

### T-Cell Transduction

1×10⁵ T-cells were added per well of a flat bottom 96-well plate. The plate was centrifuged and the supernatant aspirated before adding 50-100 μl of lentiviral supernatant supplemented with 4 μg/ml Polybrene (Hexadimethrine bromide—Sigma: H9268-5G) and IL-2 at the indicated concentration. In some instances activation reagents were added: DynabeadsTM Human T-Activator CD3/CD28 (Thermo Fisher: 11131D), DynabeadsTM Human T-Activator CD3/CD28/CD137 (Thermo Fisher 11162D) at the manufacturer recommended concentrations.

### Paramagnetic Bead Sorts

Paramagnetic bead sorts were conducted as per the manufacturers' instructions using either anti-PE microbeads (Miltenyi Biotec or StemCell Technologies), or T-cell isolation beads (17951: StemCell Technologies).

### Rapid Expansion Protocol (REP)

T-cells were expanded using irradiated buffy coat feeders. In brief 10 irradiated buffy coats were obtained from NHSBT, PBMC were isolated by Ficoll-mediated density centrifugation, mixed and cryopreserved. Thawed buffy coat feeders were mixed with T-cells at a 1:20-1:100 ratio at a final concentration of cells of 1×10⁶/ml in TCM+200 IU/ml IL-2 and 1 μg/ml phytohaemagglutinin in a T25 culture flask. The upright flask was positioned at 45° angle for the first five days after which the flask was put back upright and the media changed by half media exchange. Media exchanges were performed every 2-3 days with fresh IL-2 added to a final concentration of 200 IU/ml for 14 days after which cells were cryopreserved or put straight into assay.

### Construct Design

Previously Applicants have validated that the TpoR can have activity in primary human T-cells. However attempts to modify the receptor were not always straightforward. For example fusions between TpoR Ec domain and GCSF IC domain failed to express at the cell surface. Furthermore, Prolactin receptor fusions did not appear to be wholly surface stable. Furthermore, Applicants felt that they could improve the signalling capacity of TpoR-based receptors in T-cells by including signalling components which activate JAK3, a signalling molecule involved in IL-2 signalling but not in TpoR signalling, and therefore more likely to drive IL-2 like signals in engineered cells.

Applicants therefore aimed to generate fusion receptors wherein additional domains were fused directly to the C-terminus of the TpoR IC domain. Applicants first generated a fusion between TpoR and the IL2rβ signalling domain. Previous attempts at generating fusions between TpoR and IL2rβ by completely removing the TpoR intracellular domain resulted in receptors which did not express sufficiently well. Applicants therefore took an alternative approach where a hybrid TpoR-IL2rβ signalling domain was created whereby the I2rβ signalling region was fused N- or C-terminal to TpoR signalling domain. Next Applicants generated receptors where the cytoplasmic domain of TIAF1, TLR1, CD150, IL2rγ, CD40, LMP1 and ITAM1 from CD3ζ were fused C-terminal to the TpoR signalling domain. The reason for the choice of these receptors was as follows: TIAF1—There is evidence that TIAF1 binds JAK3 (Ji et al. 2000); TLR1/CD40—Synergy between TLRs and CD40 have been shown to induce T-cell expansion (Ahonen et al. 2004), furthermore, CD40 has been shown to bind to JAK3 and require JAK3 for signalling in B-cells (Hanissian & Geha 1997); CD150—There is evidence that CD150 may protect T-cells from IL-2 deprivation (Aversa et al. 1997); ITAM1—Applicants decided to fuse a single ITAM from CD3ζ onto the C-terminus of TpoR in an effort to induce a mitogenic response; LMP1—LMP1 from EBV virus has been shown to interact with JAK3 (Gires et al. 1999), additionally Applicants also fused LMP1 directly to the TpoR transmembrane domain as Applicants felt the TpoR cytoplasmic domain fusion would be quite large and might fail to express sufficiently well. Applicants also generated CrGFR consisting of TpoR extracellular and transmembrane domain fused to the cytoplasmic domain of CD28 and CD137 as Applicants felt these would provide a costimulatory growth signal upon Eltrombopag administration, sequences of these constructs are provided below.

The constructs were cloned into pSF.Lenti (Oxford Genetics) via an XbaI and NheI site. All fragments and constructs were codon optimised, gene synthesised and cloned by Genewiz.

Lentiviral Production—Lentiviral production was performed using a three-plasmid packaging system (Cell Biolabs, San Diego, USA) by mixing 10 μg of each plasmid, plus 10 μg of the pSF.Lenti lentiviral plasmid containing the transgene, together in serum free RPMI containing 50 mM CaCl2. The mixture was added dropwise to a 50% confluent monolayer of 293T cells in 75 cm2 flasks. The viral supernatants were collected at 48 and 72 h post transfection, pooled and concentrated using LentiPac lentiviral supernatant concentration (GeneCopoeia, Rockville, Md., USA) solution according to the manufacturer's instructions. Lentiviral supernatants were concentrated 10-fold and used to directly infect primary human T-cells in the presence of 4 μg/ml polybrene (Sigma-Aldrich, Dorset, UK).

Peripheral blood mononuclear cells were isolated from normal healthy donors before activation for 24 hours with T-cell activation and expansion beads (Invitrogen) according to the manufacturer's instructions before addition of lentiviral supernatants.

Following expansion cells were washed excessively to remove any exogenous IL2 and plated into 96-well U-bottom plates. Cells were supplemented with IL2 (Proleukin) or Eltrombopag (Stratech Scientific, Suffolk, UK). At various time points thereafter cells were either stained with a 1:400 dilution of eFlor-450 fixable viability dye (eBioscience, UK) and counted directly from the wells using a MACSQuant Cytometer, or were stained with DRAQ7 viability dye plus phycoerythrin conjugated anti-CD110 antibodies (Miltenyi Biotec, UK) and analysed using a MACSQuant cytomter. Cell viability and/or transduction level was then analysed using MACSQuantify software (Miltenyi Biotec, UK).

### Results

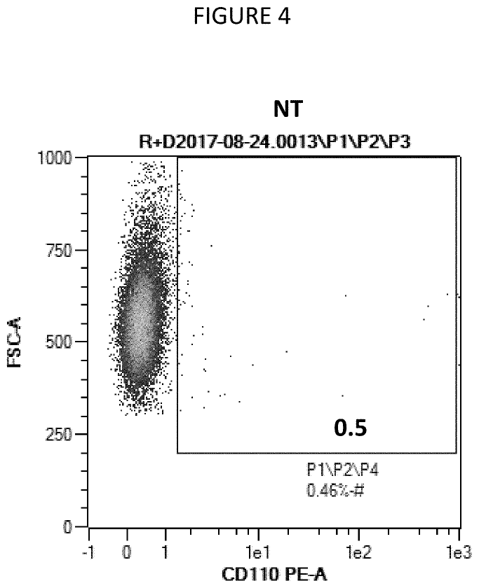

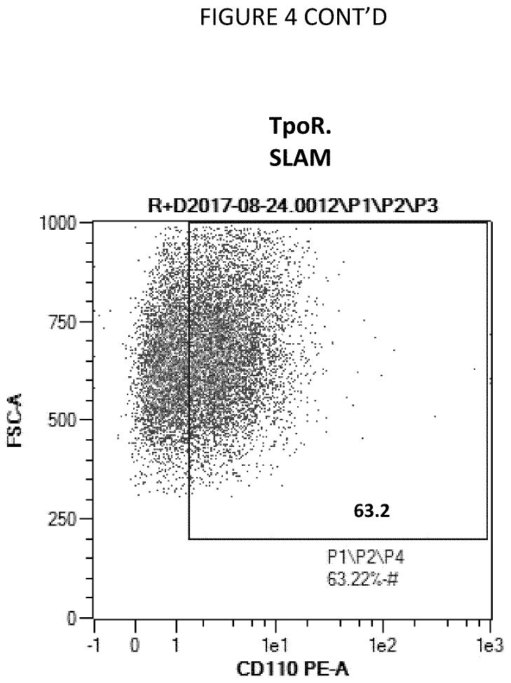

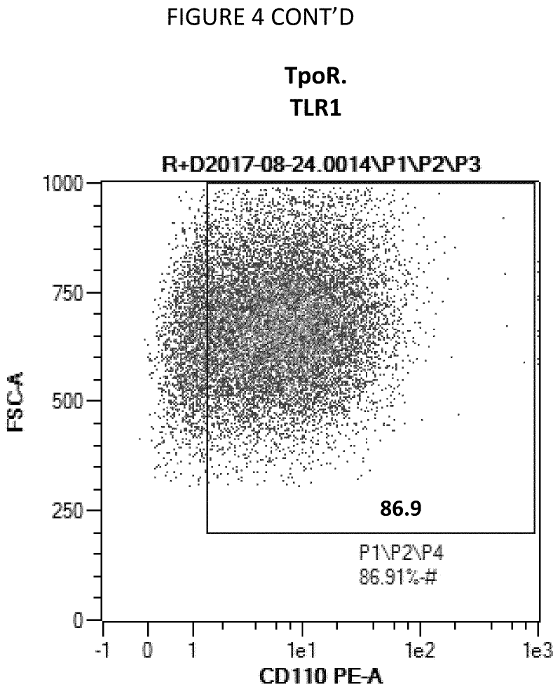

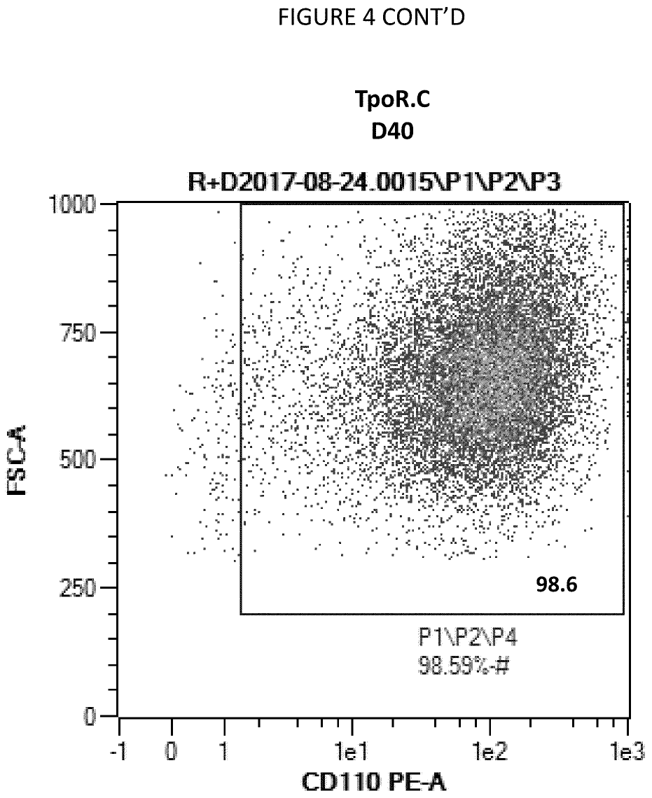

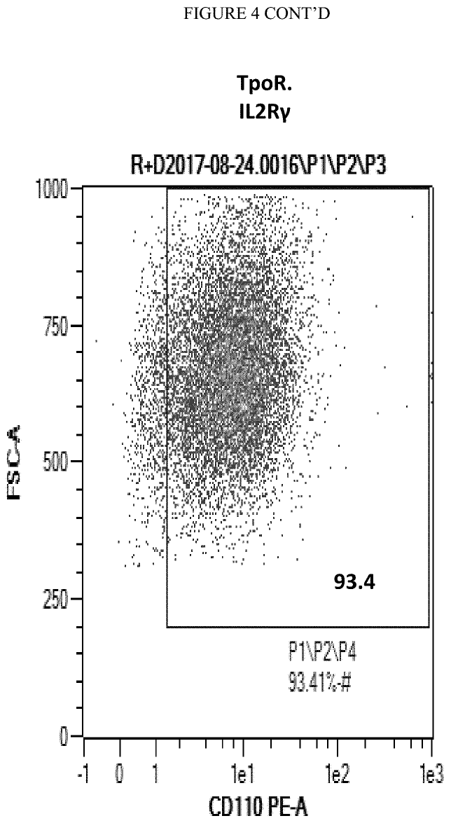

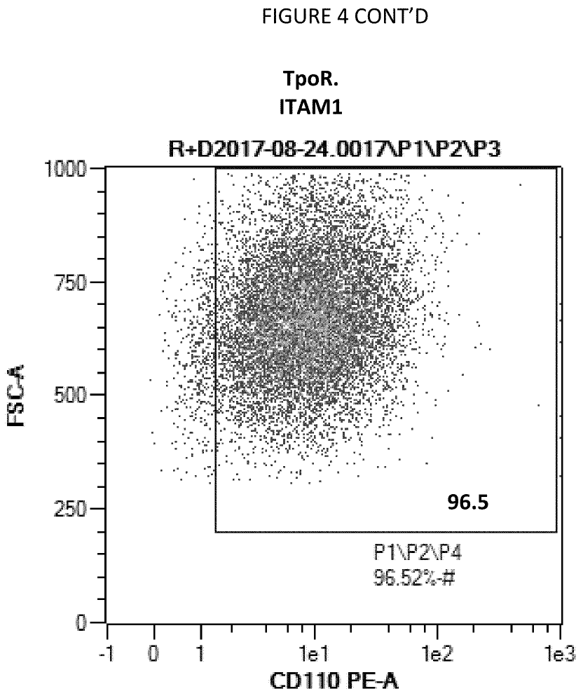

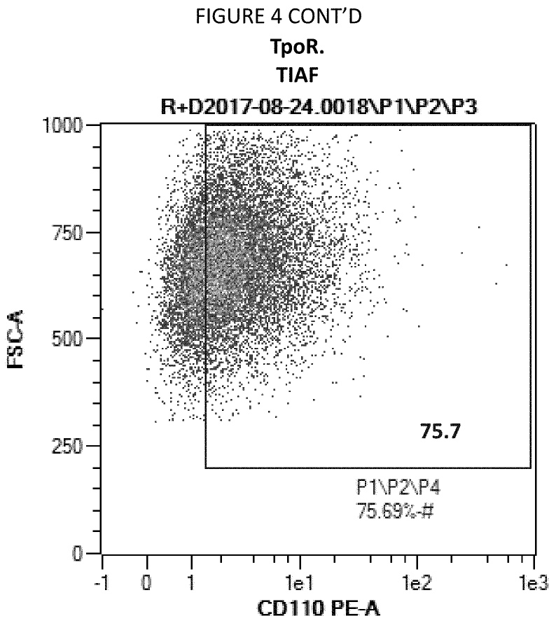

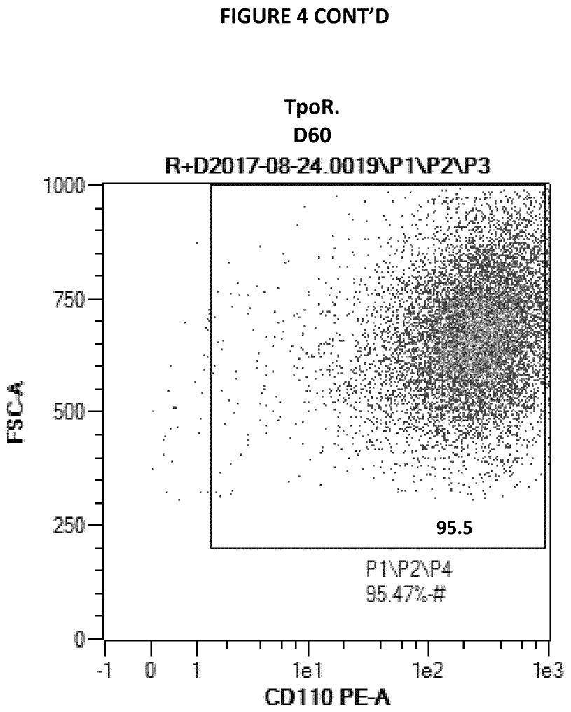

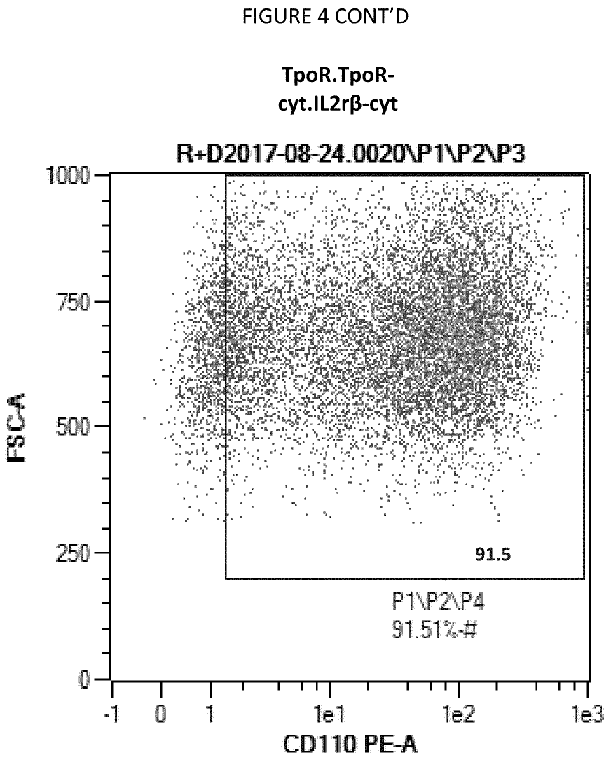

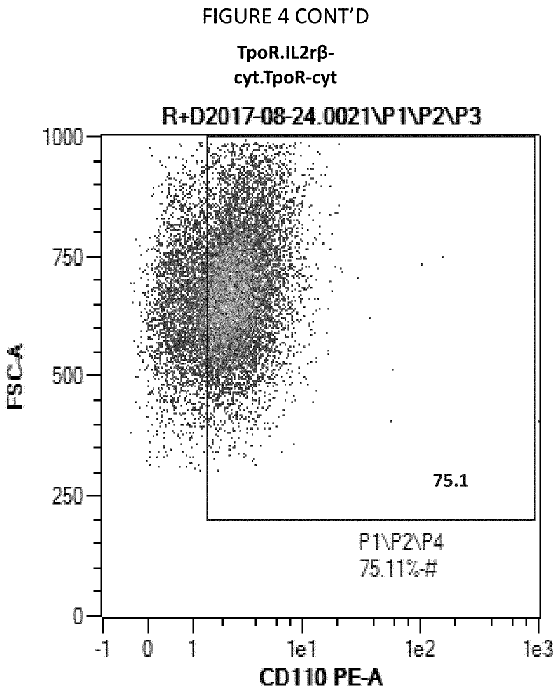

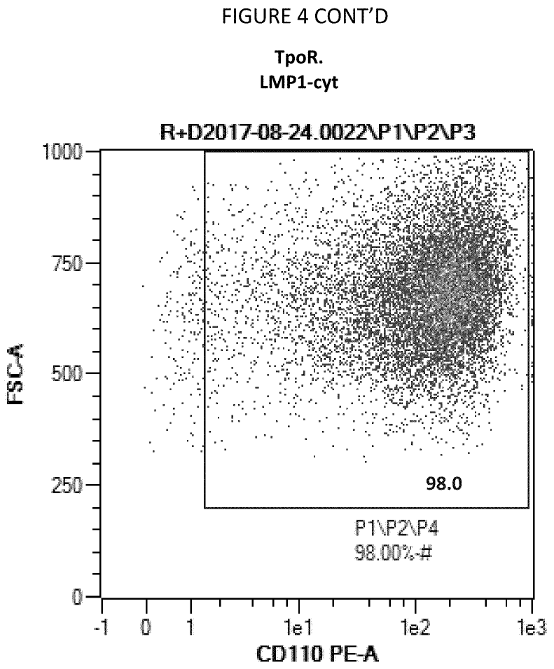

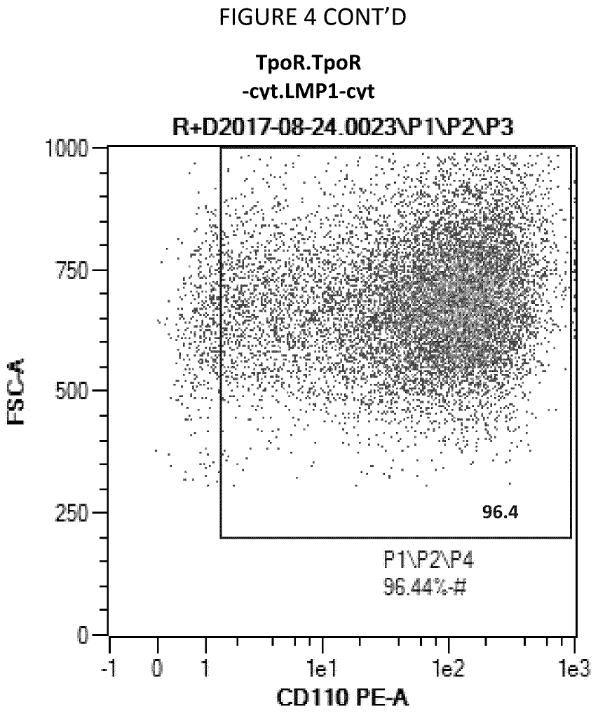

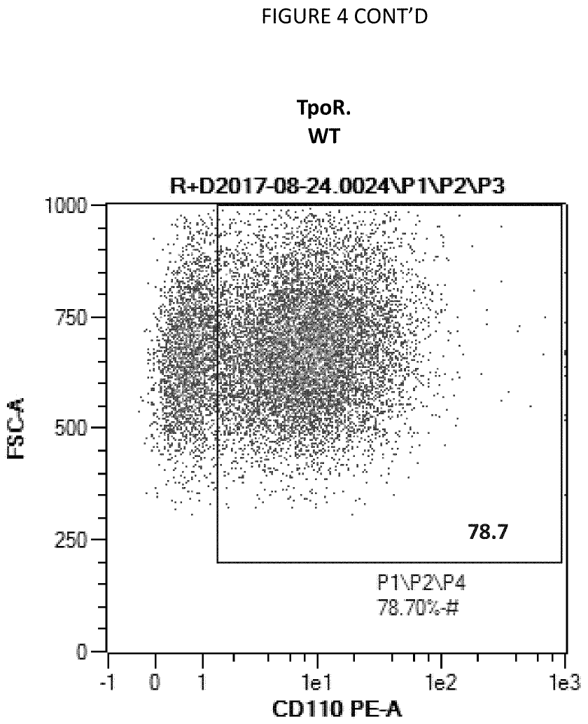

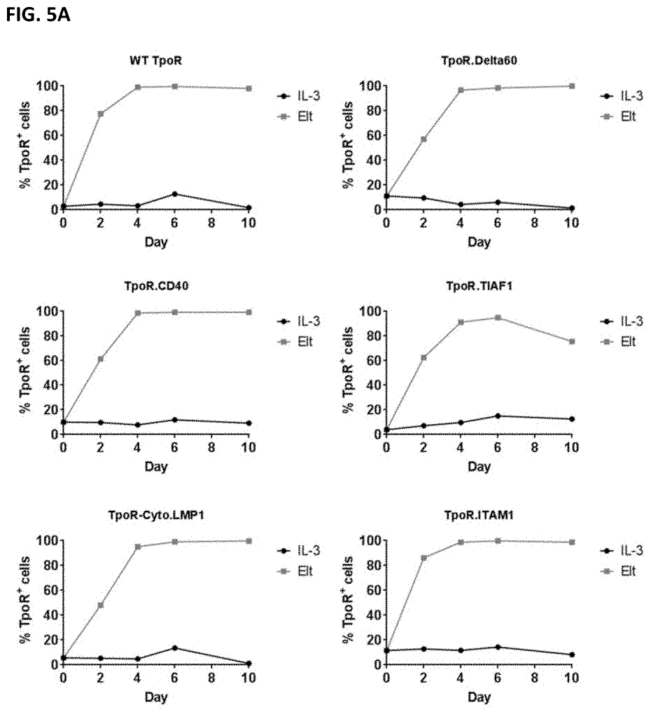

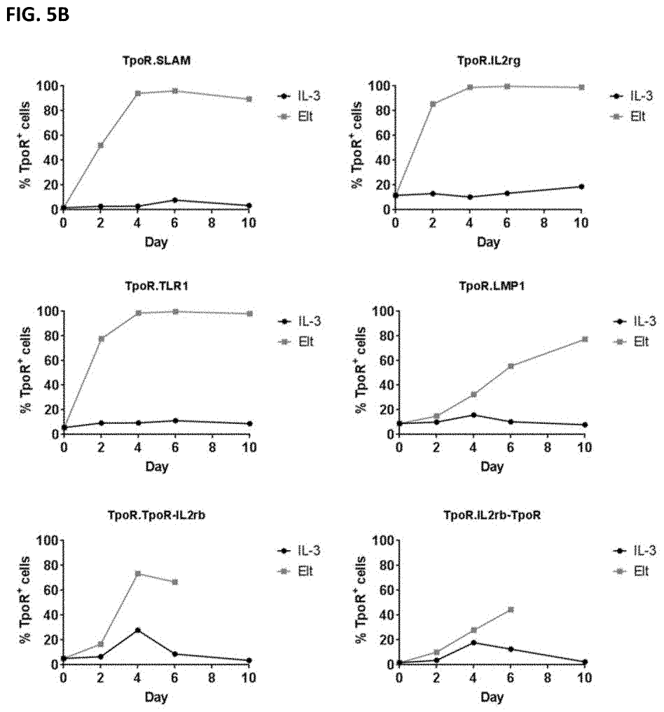

Applicants initially tested the functionality and expression profiles of the CrGFR in comparison to the wt receptor in Jurkat E6.1 and Ba/F3 cells which are human T-cell lymphoma and IL-3 dependent murine B-cell lines respectively. Although Ba/F3 are not human nor a T-cell they would at least show whether the receptors can fold properly and express, and whether they are capable of transmitting a signal. Lentiviral particles were made and used to directly infect Jurkat E6.1 and Ba/F3 cells. The Jurkat cells were analysed after 48 h for expression by use of a PE conjugated anti-CD110 antibody. Ba/F3 cells were incubated with Eltrombopag or murine IL-3 and expression of the CrGFR assessed over a number of days via analysis of CD110 expression by flow cytometry. As FIG. 4 shows all the receptors could be successfully detected in Jurkat E6.1 cells, although three receptors (TpoR.SLAM, TpoR.TIAF1 and TpoR.IL2rβ-cyt.TpoR-cyt) had a low expression profile suggesting they do not express particularly well at the surface. In Ba/F3 cells all the receptors expressed and could be enriched in the population by the addition of Eltrombopag but not IL-3 as predicted (FIG. 5). However, the two IL2rβ fusion receptors—although capable of being enriched in the population—had a poor survival profile in the Ba/F3 and the assay had to be cut short with these receptors due to a lack of viable cells.

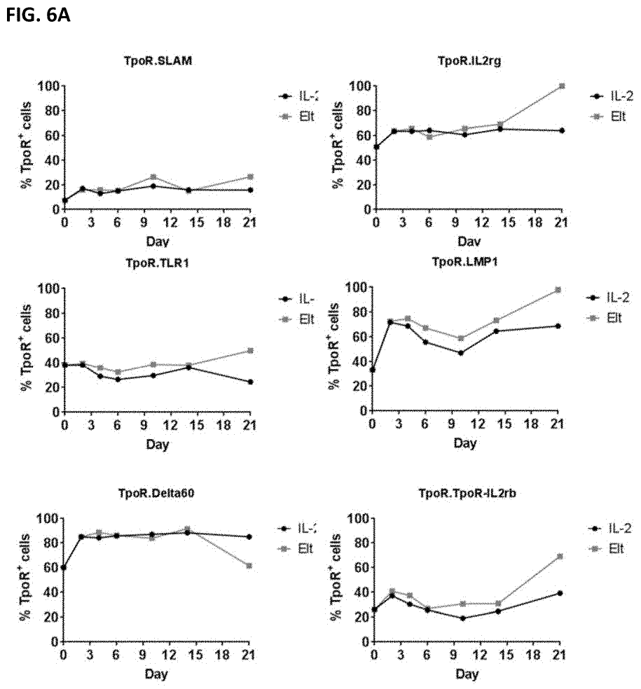

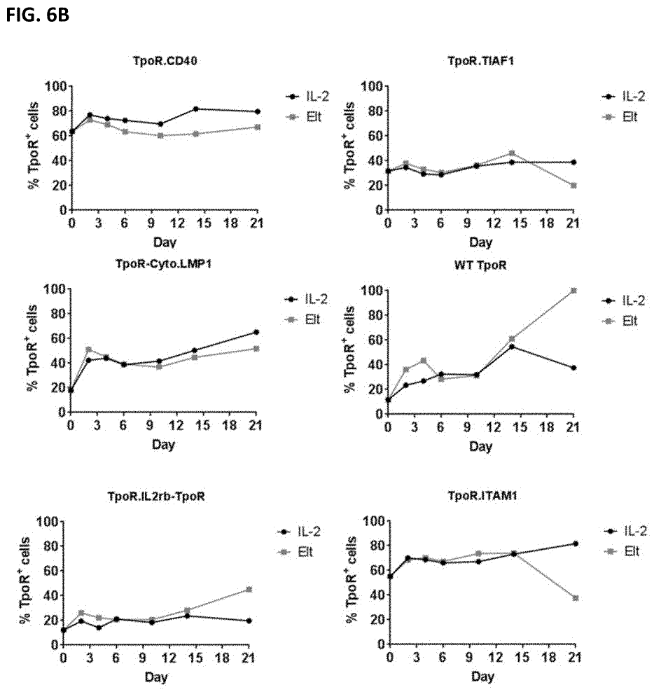

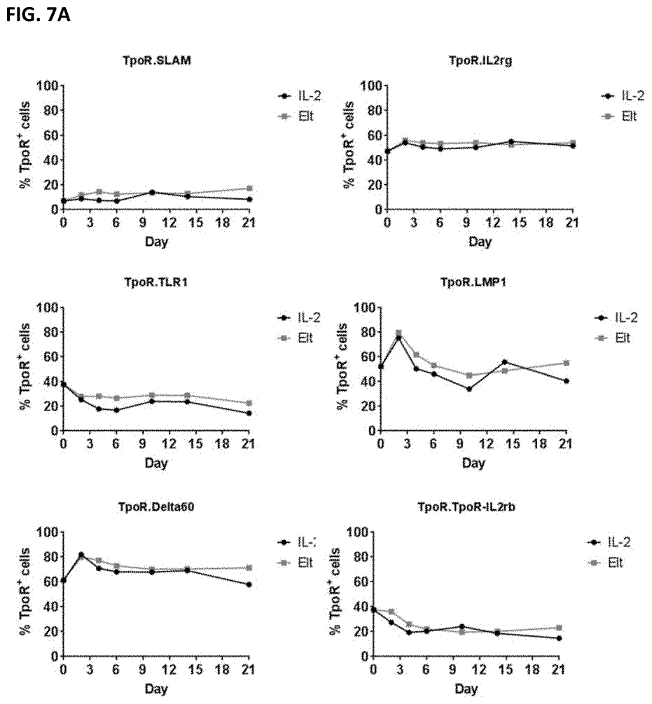

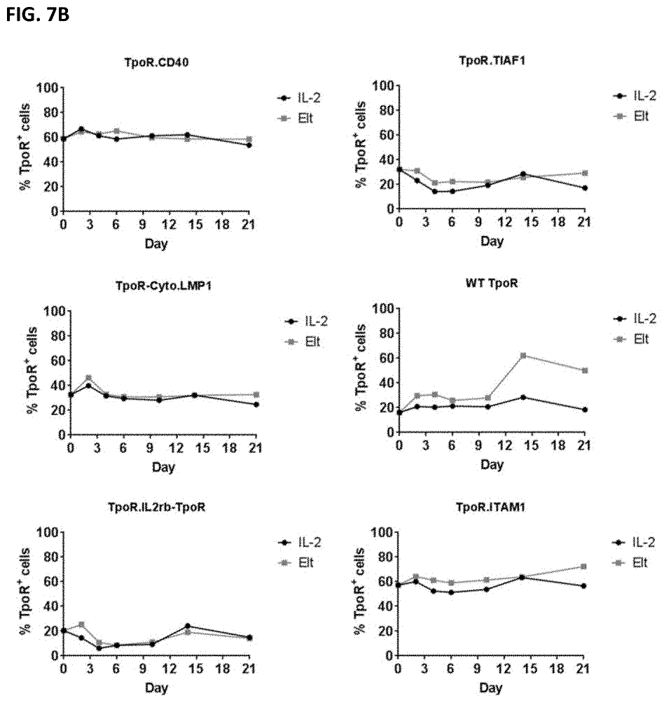

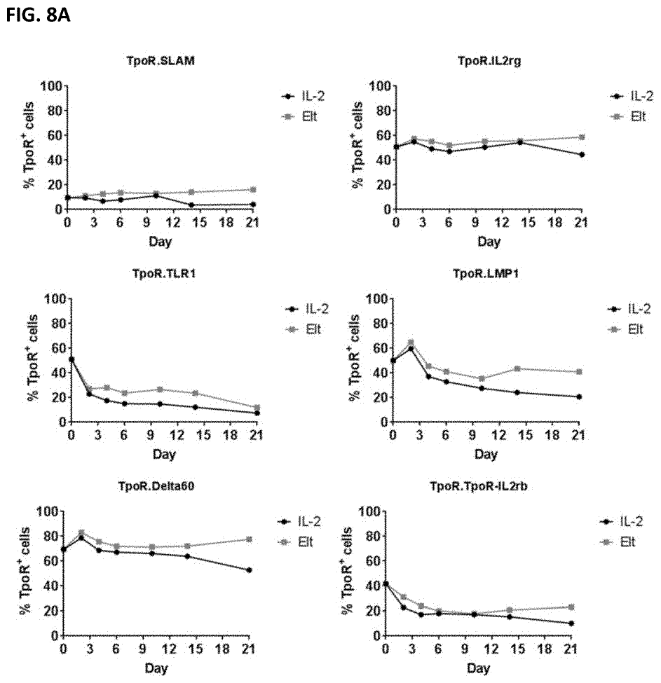

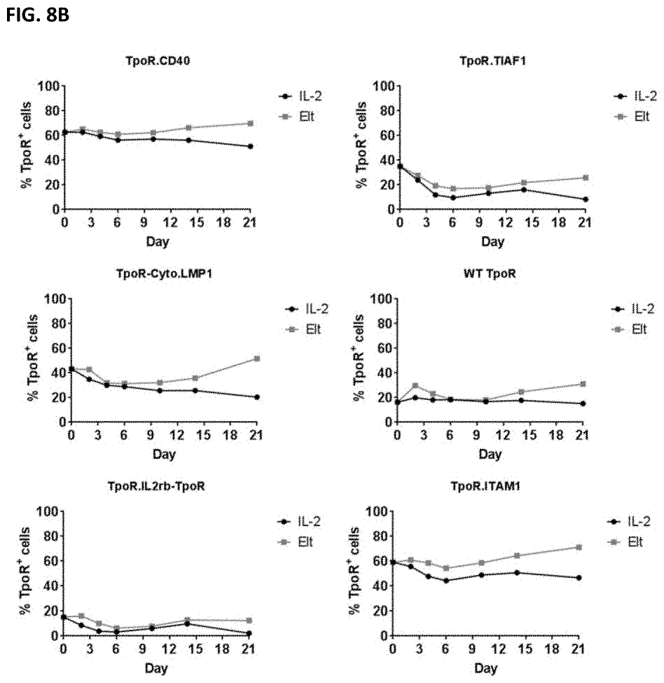

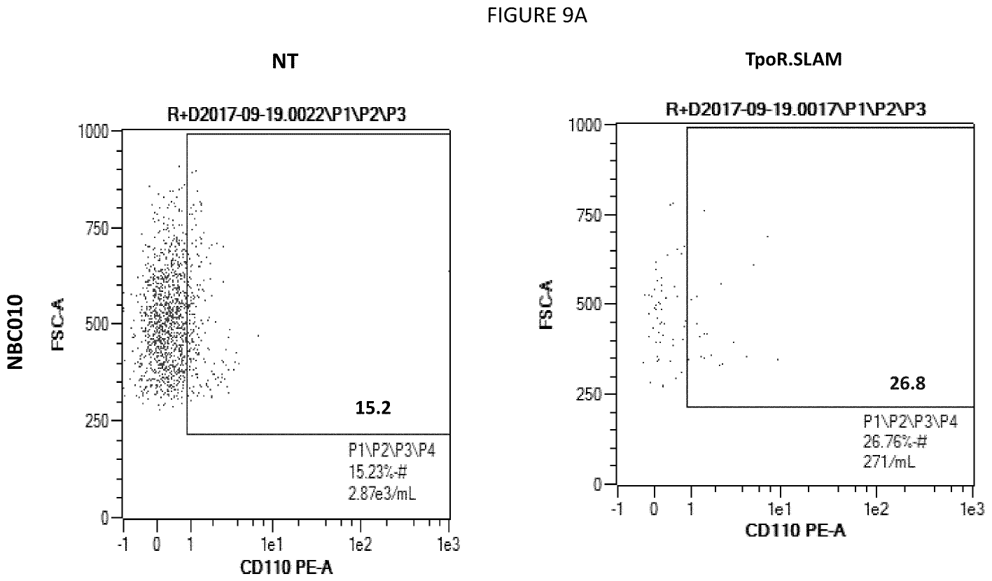

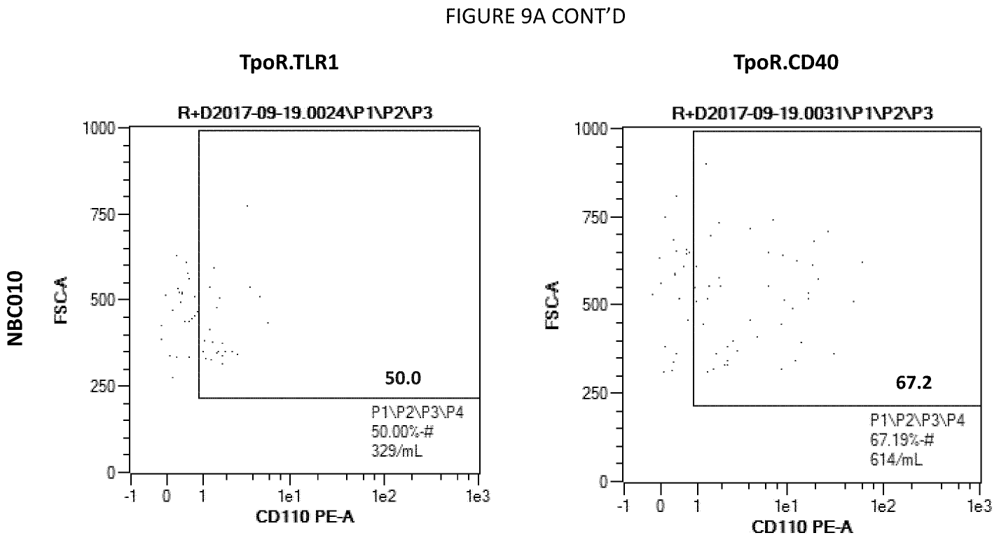

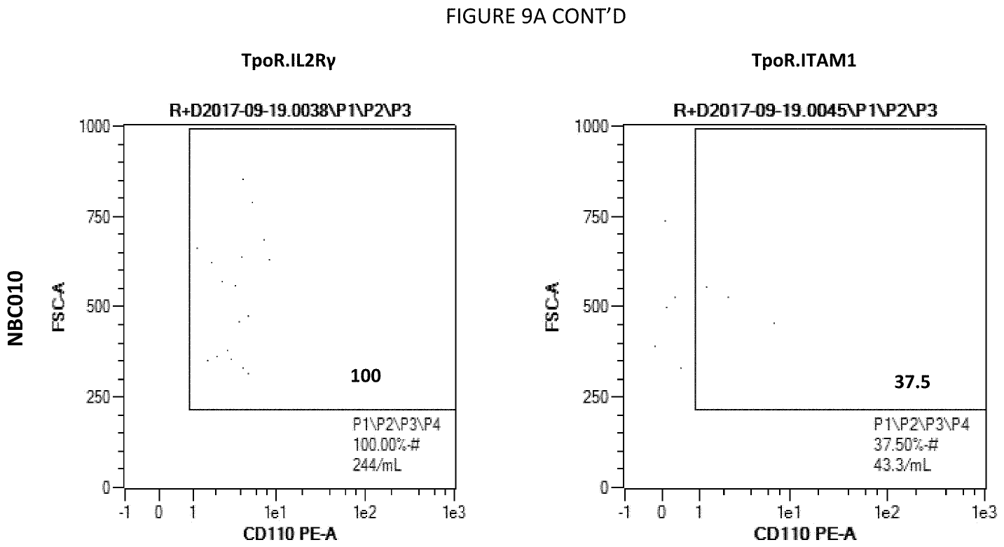

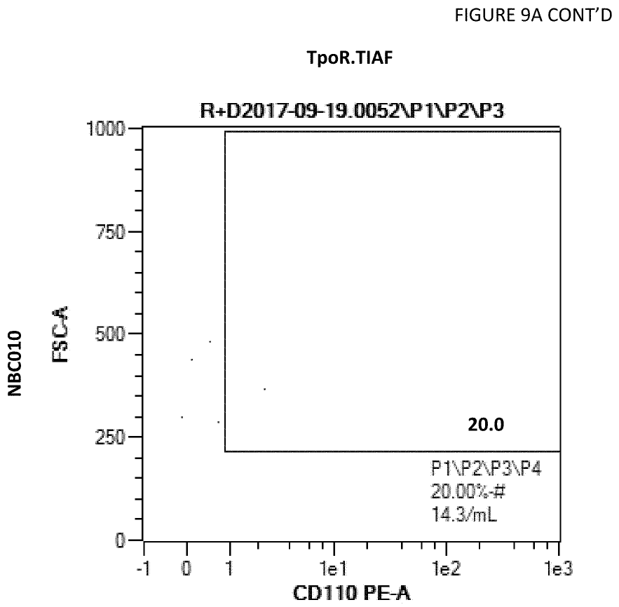

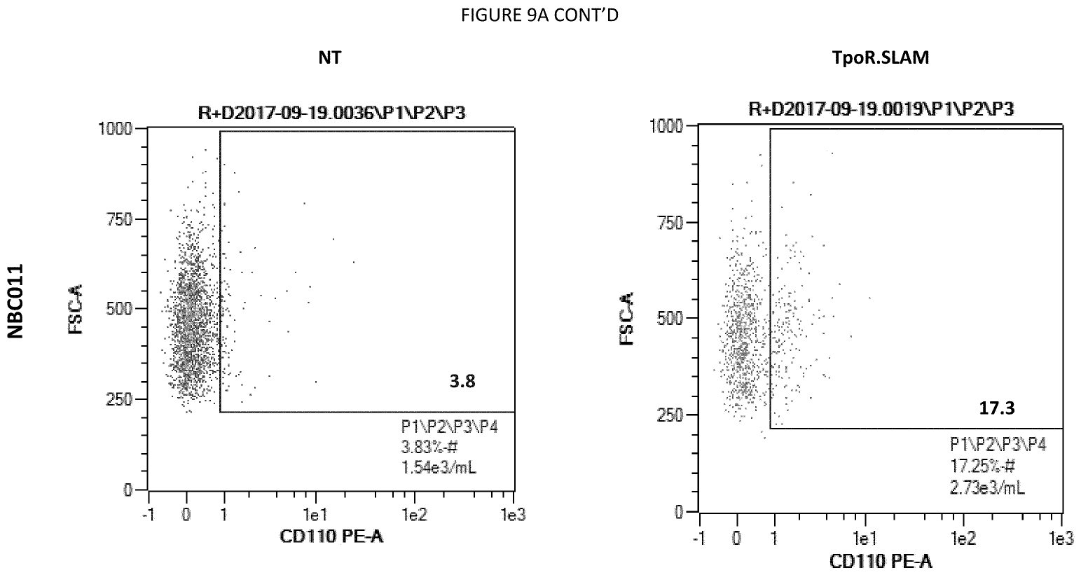

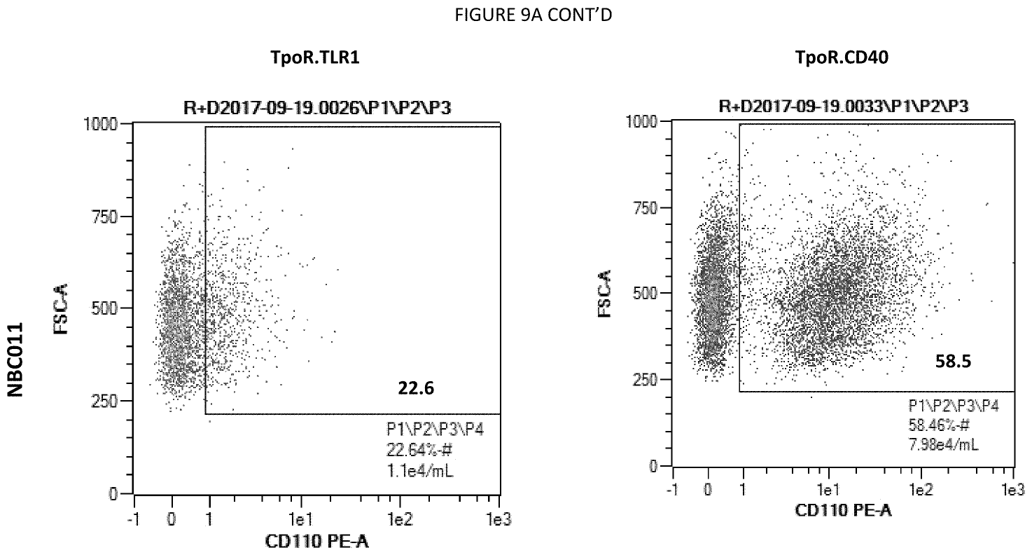

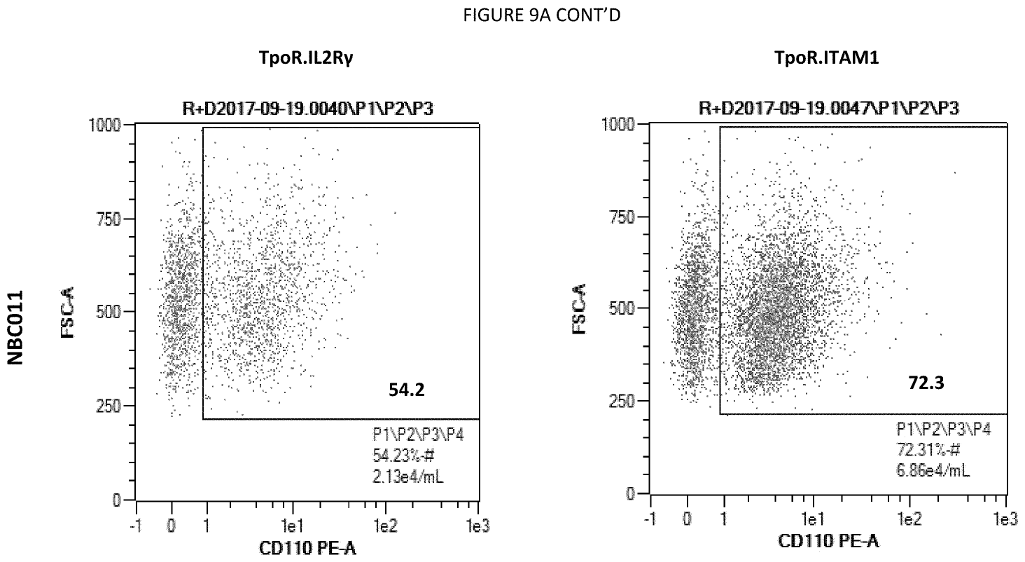

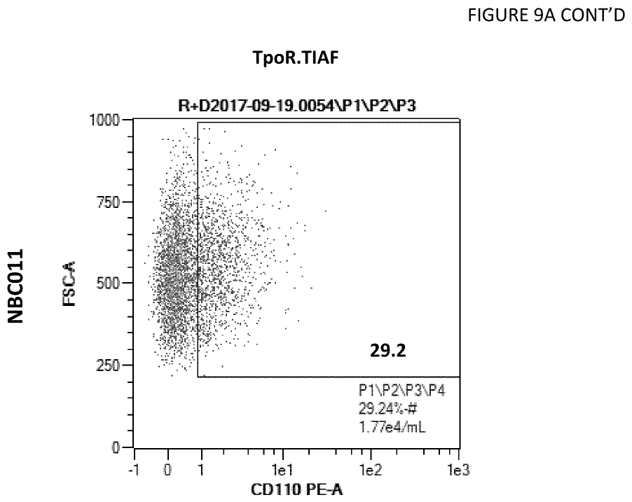

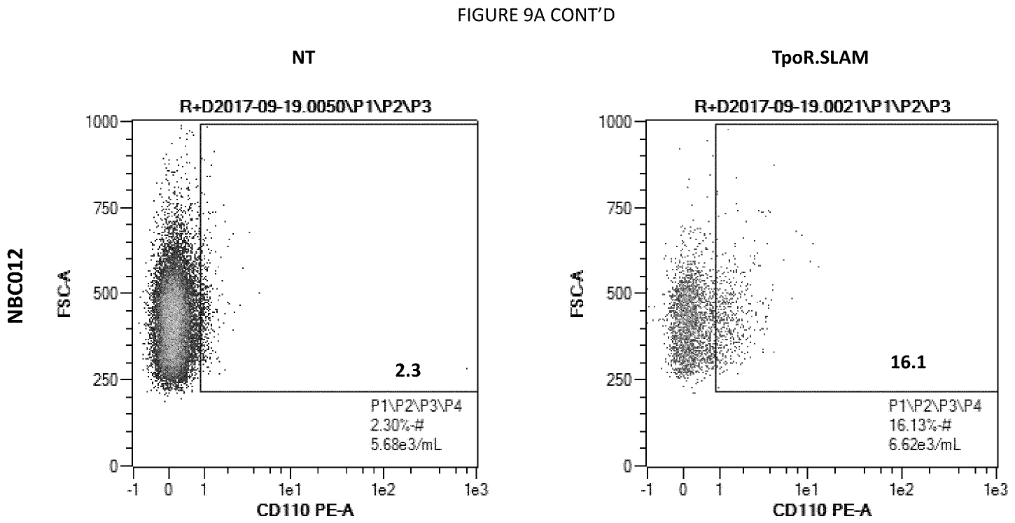

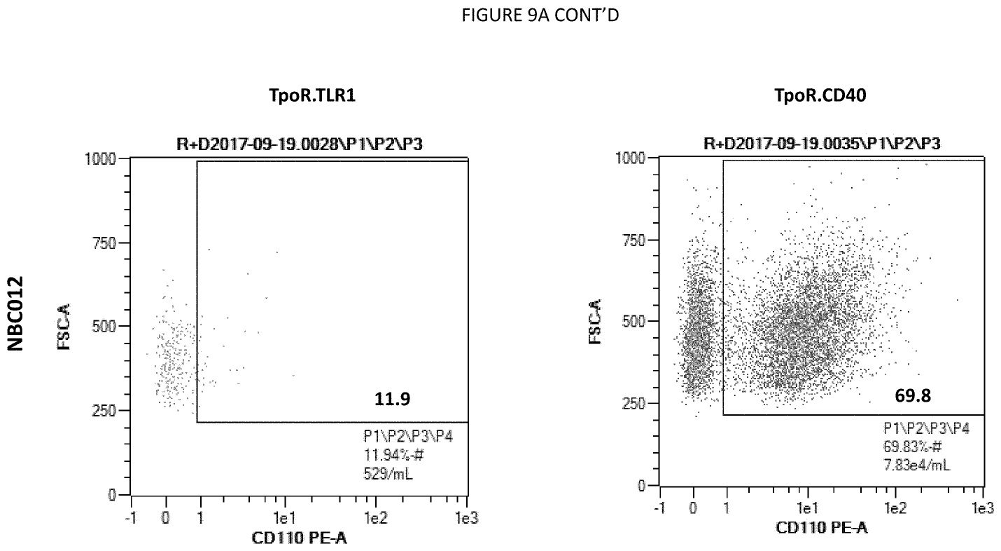

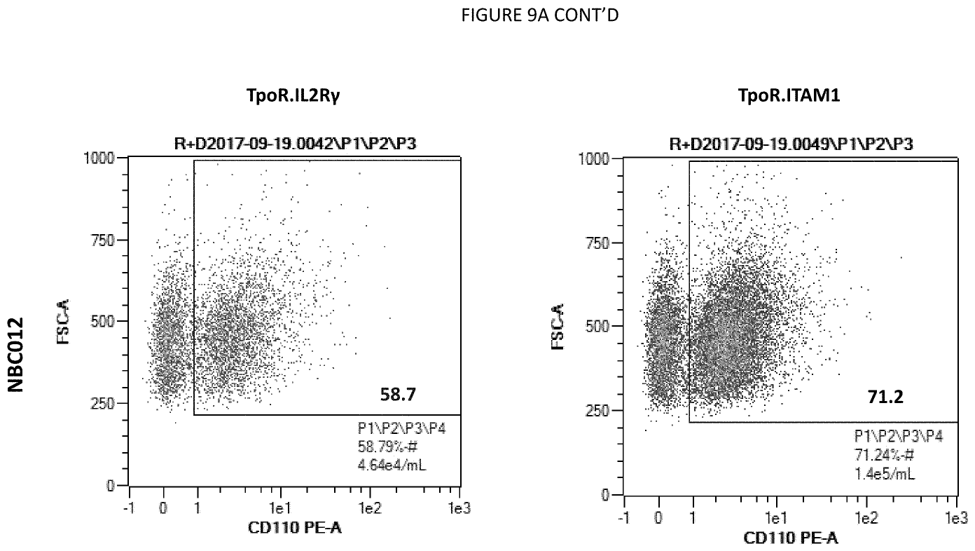

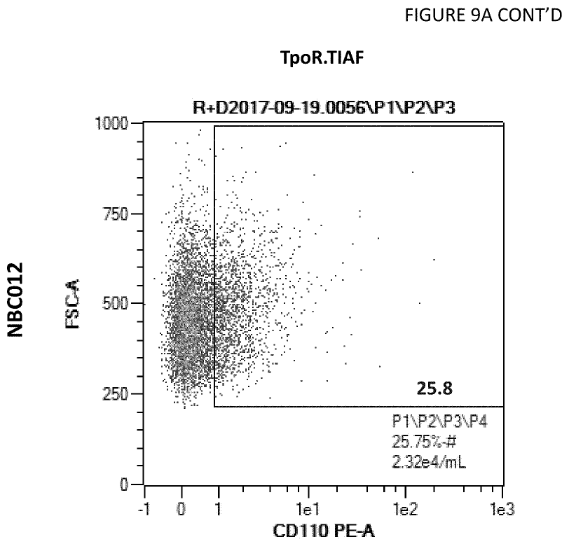

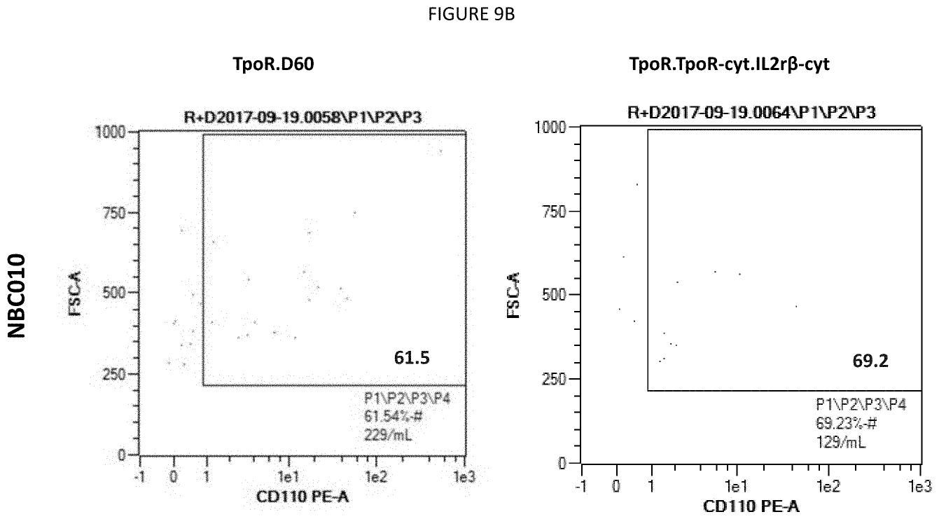

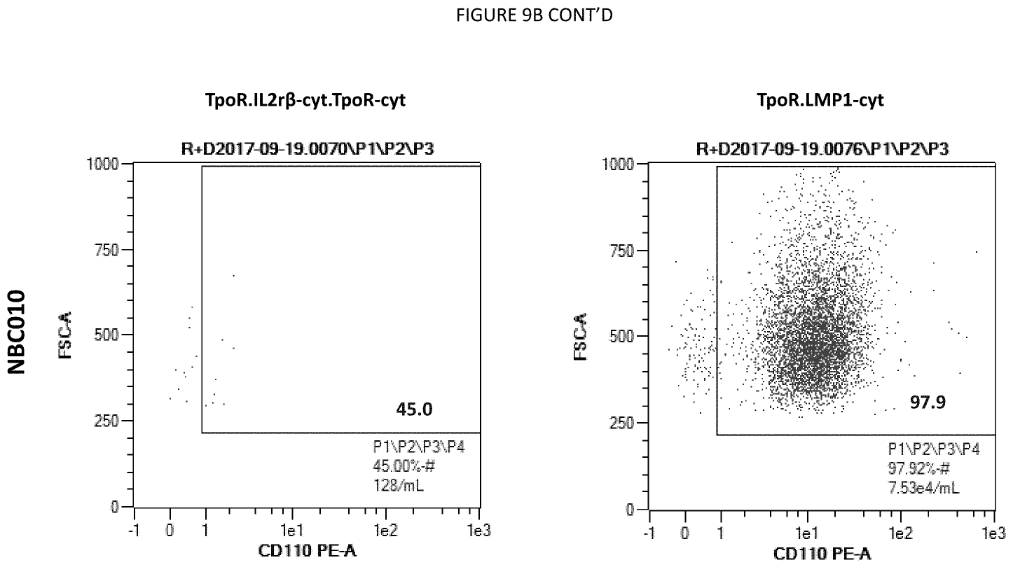

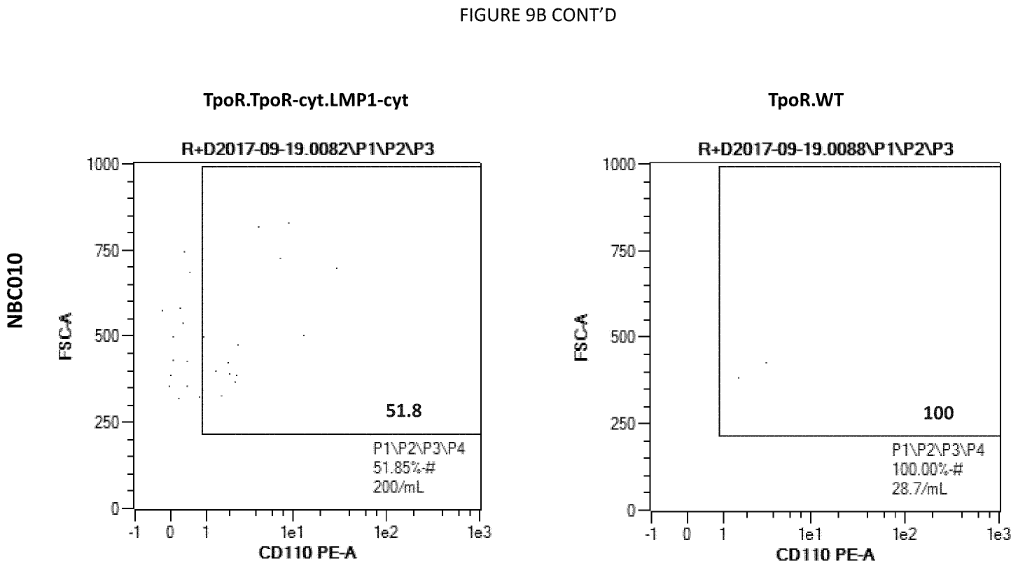

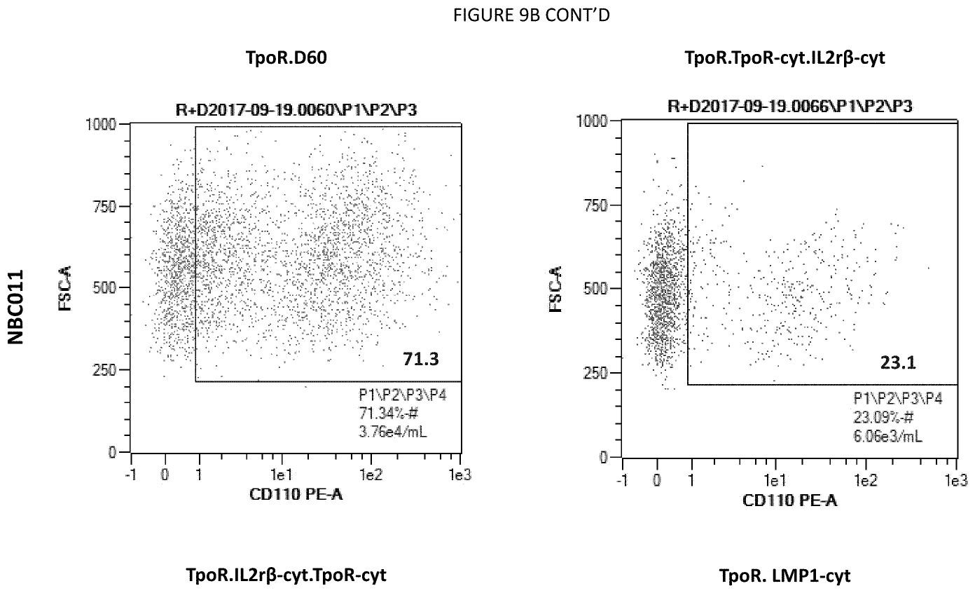

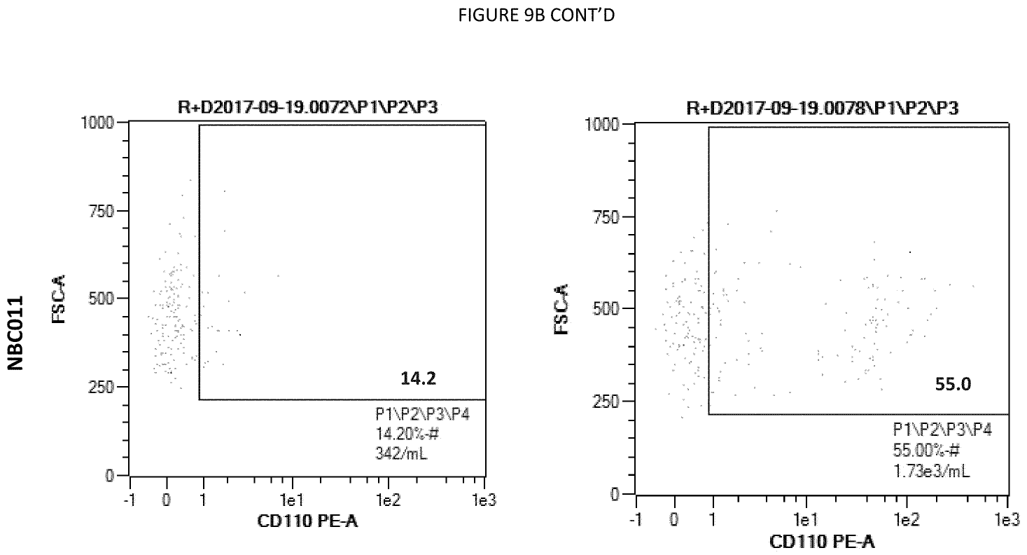

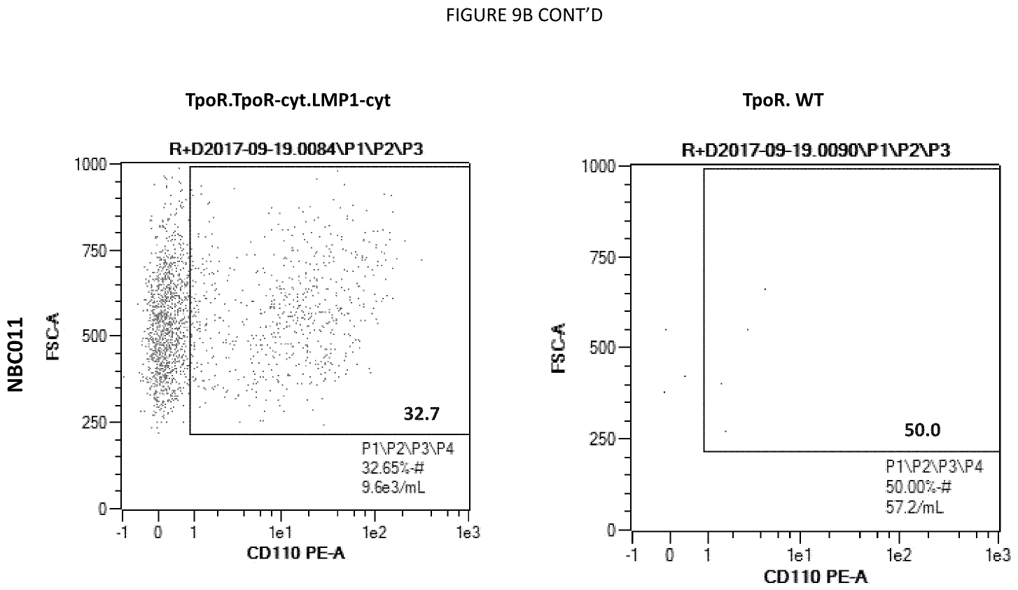

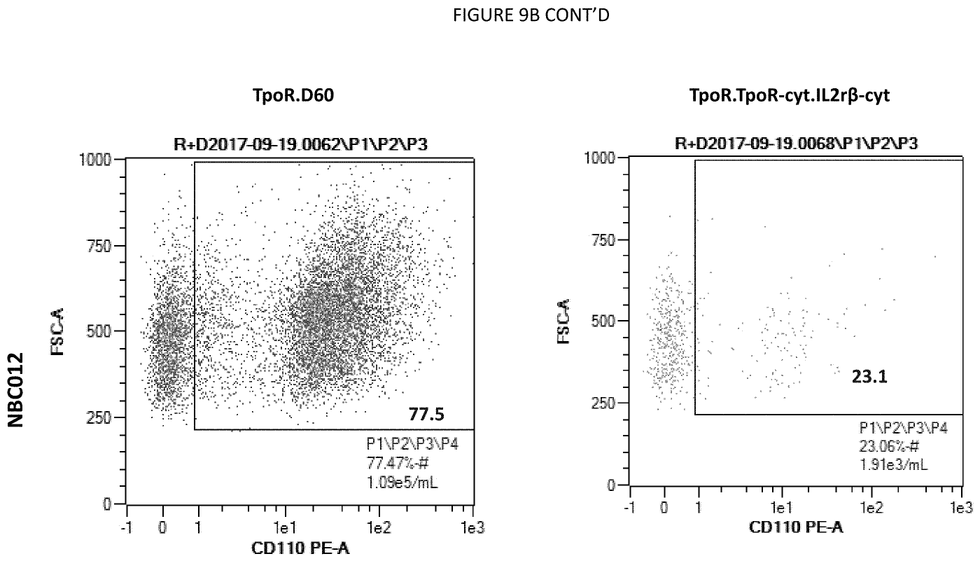

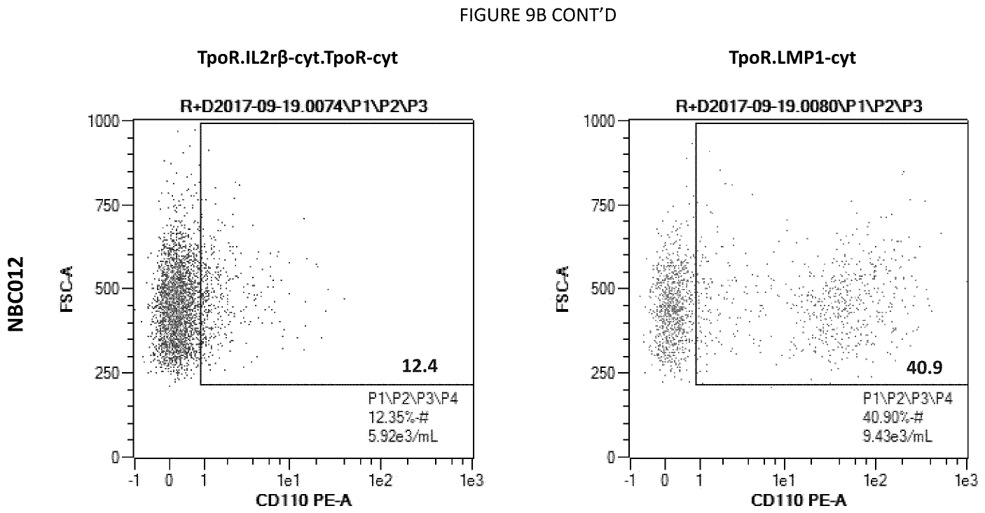

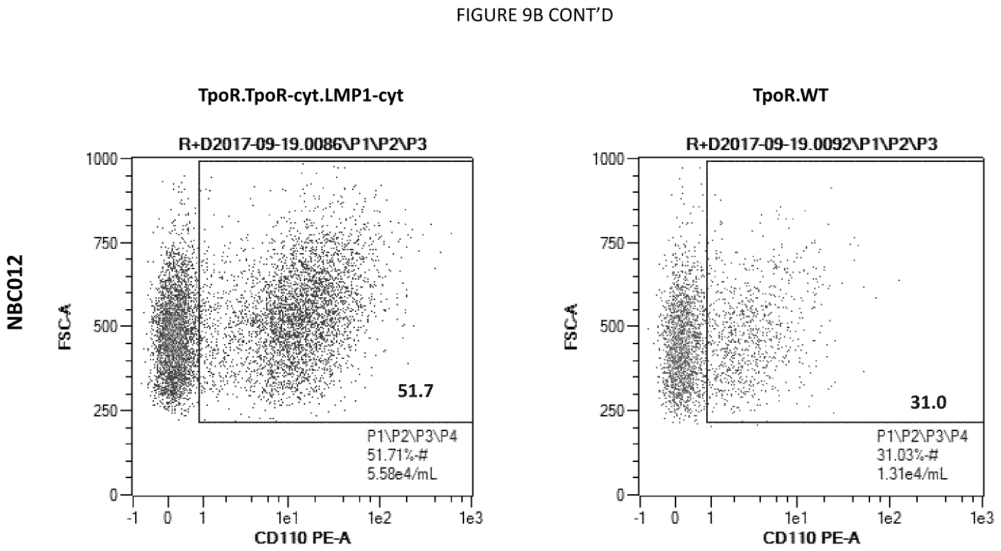

Next Applicants took these receptors and expressed them in primary human T-cells and exposed these cells to IL-2 or Eltrombopag. Three donor primary human T-cell populations were isolated from buffy coats and transduced with the indicated lentiviral constructs in the presence of CD3/CD28 Dynabeads. Following expansion the cells were incubated with IL-2 or Eltrombopag. The results are shown in FIGS. 6, 7 and 8 (×3 donors). Applicants saw an increase in expansion/survival of T-cells with some of the receptors in some of the donors. Applicants analysed this data set overall by looking at the proportion of cells expressing a good proportion of viable cells with a distinct population of CD110+ cells after 21 days. This narrowed Applicants' panel of receptors to analyse further to: TpoR.CD40, TpOR.IL2rγ, TpoR.ITAM1, TpOR.LMP1-cyt, and TpoR.TpoR-cyt-LMP1-cyt. TpoR.A60 also looked good, but Applicants did not pursue this initially with the idea this could be later incorporated into later generation fusion receptors.

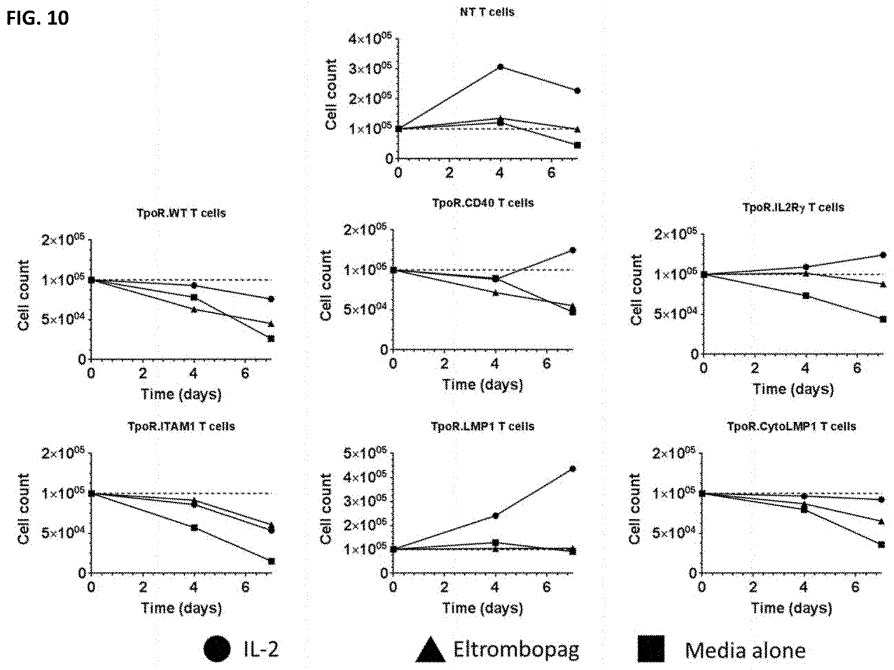

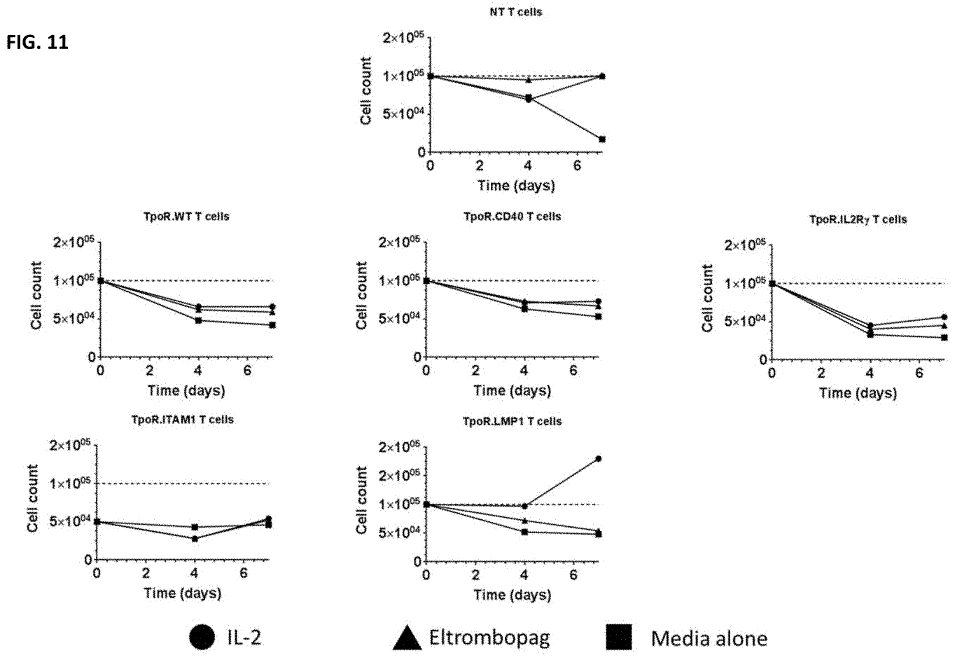

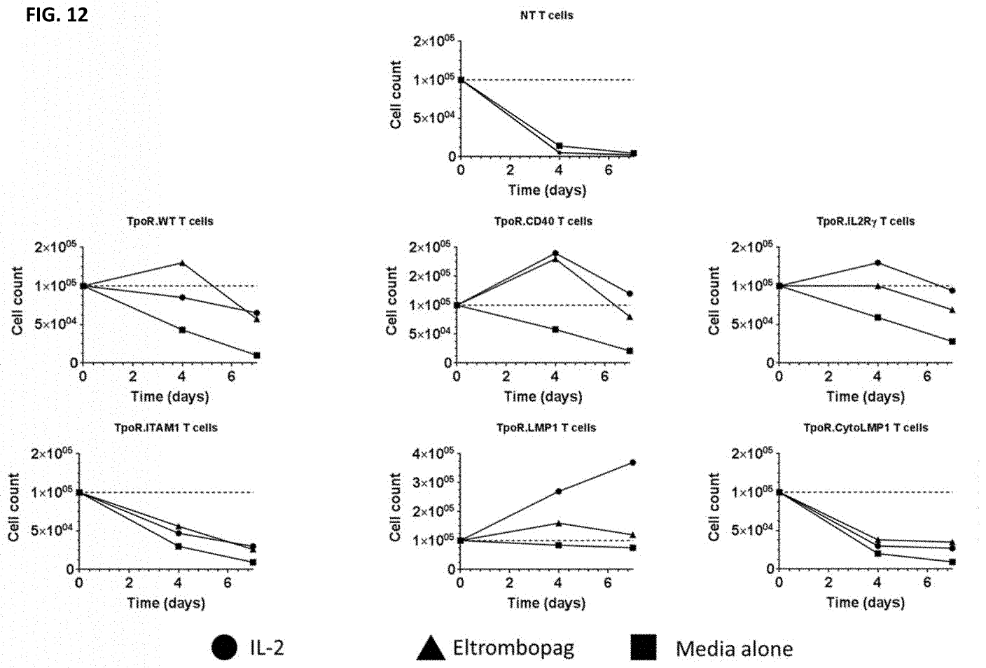

Next Applicants repeated the experiment but sorted the CrGFR+ cells using CD110+ selection by paramagnetic bead selection using the receptors identified from the first round of selections (FIGS. 10, 11 and 12). Applicants observed enhanced survival of T-cells engrafted with the majority of the CrGFR in all three donors. In particular Applicants saw expansion of WT-TpoR, TpOR.CD40, TpOR.IL2rγ and TpoR.LMP1-cyto cells in the second donor (FIG. 12) above that with media alone.

Applicants next assessed the ability of these receptors to promote survival/expansion in a model of adoptive cell therapy by engineering tumour infiltrating lymphocytes. TIL from patient TIL042 (Uveal melanoma) were engineered with the variant or wt CrGFR and mixed with patient matched tumour cells (CTUM42.1). On days 4 and 7 counts were made of the total cells as well as the CD110+ cells. Applicants found an initial decline in cell numbers, probably driven by AICD or intrinsic inhibitory factors. However between days 4 and 7 Applicants observed an increase in the numbers of CD110+ cells with all the receptors tested with Eltrombopag or Eltrombopag+low dose IL-2. The effect of the TpoR.CD40 in particular was encouraging as it demonstrated no non-specific enrichment in IL2 alone, an effect seen in with the other receptors tested.

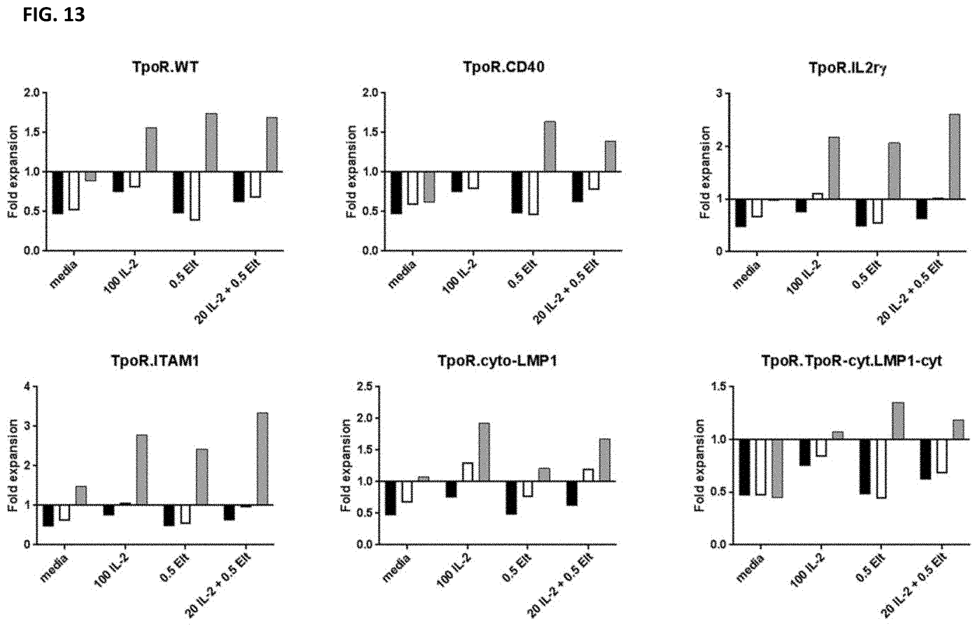

Applicants evaluated further the effect of the CrGFR in ovarian TIL. Three ovarian TIL populations were engineered to express either the WT, or TpoR.CD40, TpoR.IL2rγ or TpoR.LMP1-Cyt variant receptors and mixed with patient matched tumour cells in the presence or absence of Eltrombopag. Counts of total and CD110+ cells were made after 4 and 7 days. Applicants observed specific expansion of the CrGFR+ cells in the presence of tumour between days 4 and 7 in donors 2 and 3 with all the receptors except the TpOR.LMP1.cyt. In donor 1 Applicants found that although there was no specific expansion of CrGFR+ cells, the addition of Eltrombopag appeared to protect the cells from AICD (activation-induced cell death). Importantly Applicants found that in all three donors the activity of the TpoR.IL2rγ and TpoR.CD40 variants was superior to that of the WT receptor (FIG. 13).

Finally, Applicants validated the signalling potential of the novel CrGFR by conducting phospho STAT analysis upon treatment of CrGFR expressing T-cells with media, cytokine or drug. To this end T-cells from 4 donors were transduced with either the wt TpoR, TpoR. CD40 or TpoR.IL2rγ, enriched for CrGFR expression using paramagnetic bead selection protocols and then expanded using polyclonal stimulation. The cells were treated for four hours with media alone (RPMI), IL-2, Tpo or Eltrombopag (Elt) before methanol fixation, permeabilization and analysis using pSTAT specific antibodies. STAT molecules are the key drivers of cell signalling upon cytokine activation of cells, pSTAT5 in particular is key to IL-2 activity. Indeed Applicants saw induction of pSTAT5 upon IL-2 but not media incubation. IL-12 as a control is unable to induce STAT5 activation as observed in this experiment. Tpo and Eltrombopag in particular showed induction of STAT5 activity. This was most clearly seen with the TpoR.IL2rγ CrGFR demonstrating clear activation of the correct STAT5 activation pathway when stimulated with Eltrombopag.

### Conclusion

Growth factor receptors responsive to clinically available drugs can be transferred to T-cells by gene transfer technology and therein maintain their functional capacity to deliver cell growth/survival signals. Importantly Applicants show that as an example, TpoR-based CrGFR engrafted primary human T-cells respond to the clinically available drug Eltrombopag and expand and survive in the absence of IL-2 which is normally required for optimal T-cell growth.

Here Applicants tested a number of functional variants; based on TpoR fused to the signalling domains from a number of costimulatory or cosignalling molecules or other growth factor receptors. Applicants have shown that these receptors confer IL-2 independent growth and survival in primary human T-cells and Tumour Infiltrating Lymphocytes in the presence of the TpoR agonist Eltrombopag. In particular Applicants found that a TpoR.CD40 fusion CrGFR confers very specific Eltrombopag mediated survival/expansion of TIL and shows optimal activity in primary human T-cells.

Aspects and embodiments of the invention are also set out in the following clauses:

1. A T or NK cell comprising a chimeric recombinant growth factor receptor (CrGFR) comprising:
(i) an extracellular (EC) domain;
(ii) a thrombopoietin transmembrane (TM) domain; and
(iii) a chimeric growth factor receptor intracellular (IC) domain.
2. The T or NK cell according to clause 1 wherein binding of a ligand to the CrGFR induces proliferation of the T or NK cell.
3. The T or NK cell according to clause 2 wherein the ligand is human thrombopoietin, a thrombopoietin receptor agonist, or a tumour associated antigen.
4. The T or NK cell according to clause 3 wherein the thrombopoietin receptor agonist binds to the TM domain.
5. The T or NK cell according to clause 3 or clause 4 wherein the thrombopoietin receptor agonist is selected from Eltrombopag and Romiplostim.
6. The T or NK cell according to the preceding clauses wherein the EC domain comprises the human c-mpl EC domain.
7. The T or NK cell according to the preceding clauses wherein the EC domain comprises one or more of i) a truncated EC domain, ii) a truncated c-mpl EC domain, iii) a domain that binds to a tumour associated antigen, iv) an antibody or antibody fragment that binds to a tumour associated antigen; and v) a selection marker.
8. The T or NK cell according to the preceding clauses wherein the IC domain comprises a costimulatory, coinhibitory or cosignalling domain derived from any costimulatory, coinhibitory or cosignalling molecule such as—but not limited to—CD2, CD27, CD28, CD29, CD134, CD137, CD150, PD1 etc.
9. The T or NK cell according to the preceding clauses wherein the first IC domain is selected from: human growth hormone receptor, human prolactin receptor, human thrombopoietin receptor (c-mpl), G-CSF receptor or GM-CSF receptor.
10. The T or NK cell according to the preceding clauses wherein the additional IC domain is selected from human growth hormone receptor, human prolactin receptor, human thrombopoietin receptor (c-mpl), G-CSF receptor or GM-CSF receptor, or a costimulatory or cosignalling receptor. Additionally, the IC domain also comprises a second domain derived from one of the following (but not limited to): cytokine receptor signalling domains (e.g. IL2 receptor), Cosignalling domains (e.g. CD40), viral oncogenic proteins (e.g. LMP1), costimulatory domains (e.g. CD28, CD137, CD150 etc) or other mitogenic domains (e.g. Toll like receptors, immunoreceptor tyrosine-based activation motifs, CD3 signalling domains etc). This second domain is fused directly, or via a linker domain, to the C- or N-terminus of the TpoR IC domain.
10. The T or NK cell according to the preceding clauses having the human thrombopoietin receptor TM domain or a variant thereof having at least 80% sequence identity which binds human thrombopoietin or a thrombopoietin receptor agonist.
11. The T or NK cell according to the preceding claims, wherein the CrGFR comprises the sequence shown as SEQ ID NO: 3 or a variant thereof having at least 80% sequence identity at the protein level, or with the TpoR IC domain truncated at the C-terminus by up to 79 amino acids, or with an alternative EC domain which maintains ability to respond to a synthetic agonist drug such as Eltrombopag,
12. The T or NK cell according to the preceding claims, wherein the CrGFR comprises the sequence shown as SEQ ID NO: 4 or a variant thereof having at least 80% sequence identity at the protein level, or with the TpoR IC domain truncated by up to 79 amino acids, or with an alternative EC domain which maintains ability to respond to a synthetic agonist drug such as Eltrombopag.
13. The T or NK cell according to the preceding claims, wherein the CrGFR comprises the sequence shown as SEQ ID NO: 5 or a variant thereof having at least 80% sequence identity at the protein level, or with the TpoR IC domain truncated at the C-terminus by up to 79 amino acids, or with an alternative EC domain which maintains ability to respond to a synthetic agonist drug such as Eltrombopag.
14. The T or NK cell according to the preceding claims, wherein the CrGFR comprises the sequence shown as SEQ ID NO: 6 or a variant thereof having at least 80% sequence identity at the protein level, or with the TpoR IC domain truncated at the C-terminus by up to 79 amino acids, or with an alternative EC domain which maintains ability to respond to a synthetic agonist drug such as Eltrombopag.
15. The T or NK cell according to the preceding claims, wherein the CrGFR comprises the sequence shown as SEQ ID NO: 7 or a variant thereof having at least 80% sequence identity at the protein level, or with the TpoR IC domain truncated at the C-terminus by up to 79 amino acids, or with an alternative EC domain which maintains ability to respond to a synthetic agonist drug such as Eltrombopag,
16. The T or NK cell according to the preceding claims, wherein the CrGFR comprises the sequence shown as SEQ ID NO: 8 or a variant thereof having at least 80% sequence identity at the protein level, or with the TpoR IC domain truncated at the C-terminus by up to 79 amino acids, or with an alternative EC domain which maintains ability to respond to a synthetic agonist drug such as Eltrombopag.
17. The T or NK cell according to the preceding claims, wherein the CrGFR comprises the sequence shown as SEQ ID NO: 9 or a variant thereof having at least 80% sequence identity at the protein level, or with the TpoR IC domain truncated at the C-terminus by up to 79 amino acids, or with an alternative EC domain which maintains ability to respond to a synthetic agonist drug such as Eltrombopag.
18. The T or NK cell according to the preceding claims, wherein the CrGFR comprises the sequence shown as SEQ ID NO: 10 or a variant thereof having at least 80% sequence identity at the protein level, or with the TpoR IC domain truncated at the C-terminus by up to 79 amino acids, or with an alternative EC domain which maintains ability to respond to a synthetic agonist drug such as Eltrombopag.
19. The T or NK cell according to the preceding claims, wherein the CrGFR comprises the sequence shown as SEQ ID NO: 11 or a variant thereof having at least 80% sequence identity at the protein level, or with the TpoR IC domain truncated at the C-terminus by up to 79 amino acids, or with an alternative EC domain which maintains ability to respond to a synthetic agonist drug such as Eltrombopag.
20. The T or NK cell according to the preceding claims, wherein the CrGFR comprises the sequence shown as SEQ ID NO: 12 or a variant thereof having at least 80% sequence identity at the protein level, or with the TpoR IC domain truncated at the C-terminus by up to 79 amino acids, or with an alternative EC domain which maintains ability to respond to a synthetic agonist drug such as Eltrombopag.
21. The T or NK cell according to the preceding claims, wherein the CrGFR comprises the sequence shown as SEQ ID NO: 13 or a variant thereof having at least 80% sequence identity at the protein level, or with an alternative EC domain which maintains ability to respond to a synthetic agonist drug such as Eltrombopag.
22. The T or NK cell according to the preceding claims, wherein the CrGFR comprises the sequence shown as SEQ ID NO: 14 or a variant thereof having at least 80% sequence identity at the protein level, or with an alternative EC domain which maintains ability to respond to a synthetic agonist drug such as Eltrombopag.
23. A T or NK cell according to the preceding claims, which comprises the sequence shown in any of SEQ ID NOS: 3 to 14, or a variant thereof which has at least 80% sequence identity but retains the capacity to i) bind to human thrombopoietin, or a human thrombopoietin receptor agonist; and ii) induce cell proliferation or survival.
24. The T cell or NK cell according to any preceding clause which binds to Eltrombopag.
25. The T cell or NK cell according to any preceding clause wherein the T cell is selected from a Tumour Infiltrating Lymphocyte (TIL) a T Regulatory Cell (Treg) or a primary T cell.
26. The T cell or NK cell according to any preceding clause further comprising a recombinant T-cell receptor (TCR) and/or Chimeric Antigen Receptor (CAR).
27. A nucleic acid sequence encoding the CrGFR as defined in any preceding claim.
28. A nucleic acid sequence according to clause 27 which comprises the sequence shown as SEQ ID NOS: 17 to 28 or a variant thereof which does not alter the translated protein sequence.
29—A nucleic acid sequence according to clause 27 which comprises the sequences shown in SEQ ID 3-12 but with the IC domain shown in SEQ ID NO: 2.
30. A vector which comprises a nucleic acid sequence according to clause 27-29, or any variant thereof which does not alter the translated protein sequence.
31. A method for making a T cell or NK cell according to any of clauses 1-26, which comprises the step of introducing a nucleic acid according to clause 27-29, or vector according to clause 19-28, into a T cell or NK cell.
32. A pharmaceutical composition which comprises a vector according to clause 30 or a T or NK cell according to clauses 1-26, together with a pharmaceutically acceptable carrier, diluent or excipient.
33. A method of in-vivo cell expansion comprising administering the cells of clauses 1-26, or pharmaceutical composition of clause 32 to a subject.
34. A method of in-vivo cell expansion according to clause 33 comprising administering thrombopoietin, or a thrombopoietin receptor agonist such as Eltrombopag or Romiplostim, to a subject.
35. A T or NK cell according to any of clauses 1-26, or vector according to clause 30, for use in adoptive cell therapy.
36. A T or NK cell according to any of clauses 1-26, or vector according to clause 30, for use in a method of treating cancer.
37. A method for treating cancer which comprises the step of administering the T cell or NK cell according to any of clauses 1-26 to a subject.
38. The use of a vector according to clause 30 or the T or NK cell according to any of clauses 1-26 in the manufacture of a medicament for treating cancer.
39. Eltrombopag for use in adoptive cell therapy.
40. Eltrombopag for use in the in-vitro or in-vivo expansion of T or NK cells according to any of clauses 1-26.
41. A composition comprising a T or NK cell according to clauses 1 to 26 for use in combination with thrombopoietin or a thrombopoietin receptor agonist in the treatment of a cancer.

### REFERENCES

Ahonen C L, Doxsee C L, McGurran S M, Riter T R, Wade W F, Barth R J, Vasilakos J P, Noelle R J, Kedl R M. J Exp Med. 2004 Mar. 15; 199(6):775-84. Combined TLR and CD40 triggering induces potent CD8+ T cell expansion with variable dependence on type I IFN.
Aversa G, Chang C C, Carballido J M, Cocks B G, de Vries J E. J Immunol. 1997 May 1; 158(9):4036-44. Engagement of the signaling lymphocytic activation molecule (SLAM) on activated T cells results in IL-2-independent, cyclosporin A-sensitive T cell proliferation and IFN-gamma production.
Columbyova L, Loda M, Scadden D T. Cancer Res. 1995 Aug. 15; 55(16):3509-12. Thrombopoietin receptor expression in human cancer cell lines and primary tissues.
Grupp S A, Kalos M, Barrett D, Aplenc R, Porter D L, Rheingold S R, Teachey D T, Chew A, Hauck B, Wright J F, Milone M C, Levine B L, June C H. N Engl J Med. 2013 Apr. 18; 368(16):1509-18. Chimeric antigen receptor-modified T cells for acute lymphoid leukemia.
Erickson-Miller C L, Delorme E, Tian S S, Hopson C B, Landis A J, Valoret E I, Sellers T S, Rosen J, Miller S G, Luengo J I, Duffy K J, Jenkins J M.
Stem Cells. 2009 February; 27(2):424-30. Preclinical activity of eltrombopag (SB-497115), an oral, nonpeptide thrombopoietin receptor agonist.
Fox N E, Lim J, Chen R, Geddis A E. Exp Hematol. 2010 May; 38(5):384-91. F104S c-Mpl responds to a transmembrane domain-binding thrombopoietin receptor agonist: proof of concept that selected receptor mutations in congenital amegakaryocytic thrombocytopenia can be stimulated with alternative thrombopoietic agents.
Gires O, Kohlhuber F, Kilger E, Baumann M, Kieser A, Kaiser C, Zeidler R, Scheffer B, Ueffing M, Hammerschmidt W. EMBO J. 1999 Jun. 1; 18(11):3064-73. Latent membrane protein 1 of Epstein-Barr virus interacts with JAK3 and activates STAT proteins.
Hanissian S H, Geha R S. Immunity. 1997 April; 6(4):379-87. Jak3 is associated with CD40 and is critical for CD40 induction of gene expression in B cells.
Kawahara M, Kimura H, Ueda H, Nagamune T. Biochem Biophys Res Commun. 2004 Feb. 27; 315(1):132-8. Selection of genetically modified cell population using hapten-specific antibody/receptor chimera.
Kochenderfer J N, Dudley M E, Kassim S H, Somerville R P, Carpenter R O, Stetler-Stevenson M, Yang J C, Phan G Q, Hughes M S, Sherry R M, Raffeld M, Feldman S, Lu L, Li Y F, Ngo L T, Goy A, Feldman T, Spaner D E, Wang M L, Chen C C, Kranick S M, Nath A, Nathan D A, Morton K E, Toomey M A, Rosenberg S A. J Clin Oncol. 2015 33(6):540-9. Chemotherapy-refractory diffuse large B-cell lymphoma and indolent B-cell malignancies can be effectively treated with autologous T cells expressing an anti-CD19 chimeric antigen receptor.
Jin L, Zeng H, Chien S, Otto K G, Richard R E, Emery D W, Blau C A. Nat Genet. 2000 September; 26(1):64-6. In vivo selection using a cell-growth switch.
Ji H, Zhai Q, Zhu J, Yan M, Sun L, Liu X, Zheng Z. A novel protein MAJN binds to Jak3 and inhibits apoptosis induced by IL-2 deprival. Biochem Biophys Res Commun. 2000 Apr. 2; 270(1):267-71.
Kawahara M, Chen J, Sogo T, Teng J, Otsu M, Onodera M, Nakauchi H, Ueda H, Nagamune T. Cytokine. 2011 September; 55(3):402-8. Growth promotion of genetically modified hematopoietic progenitors using an antibody/c-Mpl chimera.
Morgan R A, Dudley M E, Wunderlich J R, Hughes M S, Yang J C, Sherry R M, Royal R E, Topalian S L, Kammula U S, Restifo N P, Zheng Z, Nahvi A, de Vries C R, Rogers-Freezer L J, Mavroukakis S A, Rosenberg S A. Science. 2006 Oct. 6; 314(5796):126-9. Cancer regression in patients after transfer of genetically engineered lymphocytes.
Nagashima T, Ueda Y, Hanazono Y, Kume A, Shibata H, Ageyama N, Terao K, Ozawa K, Hasegawa M. J Gene Med. 2004 January; 6(1):22-31. In vivo expansion of gene-modified hematopoietic cells by a novel selective amplifier gene utilizing the erythropoietin receptor as a molecular switch.
Nishimura C D, Brenner D A, Mukherjee M, Hirsch R A, Ott L, Wu M F, Liu H, Dakhova O, Orange J S, Brenner M K, Lin C Y, Arber C. Blood. 2017 Dec. 21; 130(25):2739-2749. c-A/IPL provides tumor-targeted T-cell receptor-transgenic T cells with costimulation and cytokine signals.
Rapoport A P, Stadtmauer E A, Binder-Scholl G K, Goloubeva O, Vogl D T, Lacey S F, Badros A Z, Garfall A, Weiss B, Finklestein J, Kulikovskaya I, Sinha S K, Kronsberg S, Gupta M, Bond S, Melchiori L, Brewer J E, Bennett A D, Gerry A B, Pumphrey N J, Williams D, Tayton-Martin HK, Ribeiro L, Holdich T, Yanovich S, Hardy N, Yared J, Kerr N, Philip S, Westphal S, Siegel D L, Levine B L, Jakobsen B K, Kalos M, June C H. Nat Med. 2015 August; 21(8):914-21. NY-ESO-1-specific TCR-engineered T cells mediate sustained antigen-specific antitumor effects in myeloma.
Richard R E, Wood B, Zeng H, Jin L, Papayannopoulou T, Blau C A. Blood. 2000 Jan. 15; 95(2):430-6. Expansion of genetically modified primary human hemopoietic cells using chemical inducers of dimerization.
Rosenberg S A, Yang J C, Sherry R M, Kammula U S, Hughes M S, Phan G Q, Citrin D E, Restifo N P, Robbins P F, Wunderlich J R, Morton K E, Laurencot C M, Steinberg S M, White D E, Dudley M E. Clin Cancer Res. 2011 Jul. 1; 17(13):4550-7. Durable complete responses in heavily pretreated patients with metastatic melanoma using T-cell transfer immunotherapy.
Saka K, Kawahara M, Teng J, Otsu M, Nakauchi H, Nagamune T. J Biotechnol. 2013 December; 168(4):659-65. Top-down motif engineering of a cytokine receptor for directing ex vivo expansion of hematopoietic stem cells.
Saka K, Kawahara M, Ueda H, Nagamune T. Biotechnol Bioeng. 2012 June; 109(6):1528-37. Activation of target signal transducers utilizing chimeric receptors with signaling-molecule binding motifs.
Yamane N, Tanaka Y, Ohyabu N, Yamane S, Maekawa K, Ishizaki J, Suzuki R, Itoh T, Takemoto H. Eur J Pharmacol. 2008 May 31; 586(1-3):44-51. Characterization of novel non-peptide thrombopoietin mimetics, their species specificity and the activation mechanism of the thrombopoietin receptor.

### Sequences

In the amino acid sequences below, bold indicates TpoR derived sequence.

In the nucleotide sequences below, degenerate bases are indicated using the standard IUPAC code:

| IUPAC | | IUPAC | |
|---|---|---|---|
| nucleotide code | Base | nucleotide code | Base |
| A | Adenine | K | G or T |
| C | Cytosine | M | A or C |
| G | Guanine | B | C or G or T |
| T (or U) | Thymine (or Uracil) | D | A or G or T |
| R | A or G | H | A or C or T |
| Y | C or T | V | A or C or G |
| S | G or C | N | any base |
| W | A or T | . or - | gap |

** denotes Stop codons

Transmembrane domain underlined (in SEQ ID NOS: 1 to 15).

SEQ ID NO: 1: Wild type TpoR.

635 amino acids presented in the N- to C-terminus direction, of which 1-491 (bold): TpoR extracellular domain, 492-513 (bold, underlined): TpoR TM domain, 514-635 (bold, italics): TpoR cytoplasmic domain.

MPSWALFMVTSCLLLAPQNLAQVSSQDVSLLASDSEPLKCFSRTFEDLT
CFWDEEEAAPSGTYQLLYAYPREKPRACPLSSQSMPHFGTRYVCQFPDQ
EEVRLFFPLHLWVKNVFLNQTRTQRVLFVDSVGLPAPPSIIKAMGGSQP
GELQISWEEPAPEISDFLRYELRYGPRDPKNSTGPTVIQLIATETCCPA
LQRPHSASALDQSPCAQPTMPWQDGPKQTSPSREASALTAEGGSCLISG
LQPGNSYWLQLRSEPDGISLGGSWGSWSLPVTVDLPGDAVALGLQCFTL
DLKNVTCQWQQQDHASSQGFFYHSRARCCPRDRYPIWENCEEEEKTNPG
LQTPQFSRCHFKSRNDSIIHILVEVTTAPGTVHSYLGSPFWIHQAVRLP
TPNLHWREISSGHLELEWQHPSSWAAQETCYQLRYTGEGHQDWKVLEPP
LGARGGTLELRPRSRYRLQLRARLNGPTYQGPWSSWSDPTRVETATETA
WISLVTALHLVLGLSAVLGLLLL

[symbol]

[symbol]

[symbol]

[symbol]

[symbol]

[symbol]

[symbol]

[symbol]

[symbol]

**
SEQ ID NO: 15: Wild type TpoR
atgccnwsntgggcnytnttyatggtnacnwsntgyytnytnytngcnc
cncaraayytngcncargtnwsnwsncargaygtnwsnytnytngcnws
ngaywsngarccnytnaartgyttywsnmgnacnttygargayytnacn
tgyttytgggaygargargargcngcnccnwsnggnacntaycarytny
tntaygcntayccnmgngaraarccnmgngcntgyccnytnwsnwsnca
rwsnatgccncayttyggnacnmgntaygtntgycarttyccngaycar
gargargtnmgnytnttyttyccnytncayytntgggtnaaraaygtnt
tyytnaaycaracnmgnacncarmgngtnytnttygtngaywsngtngg
nytnccngcnccnccnwsnathathaargcnatgggnggnwsncarccn
ggngarytncarathwsntgggargarccngcnccngarathwsngayt
tyytnmgntaygarytnmgntayggnccnmgngayccnaaraaywsnac
nggnccnacngtnathcarytnathgcnacngaracntgytgyccngcn
ytncarmgnccncaywsngcnwsngcnytngaycarwsnccntgygcnc
arccnacnatgccntggcargayggnccnaarcaracnwsnccnwsnmg
ngargcnwsngcnytnacngcngarggnggnwsntgyytnathwsnggn
ytncarccnggnaaywsntaytggytncarytnmgnwsngarccngayg
gnathwsnytnggnggnwsntggggnwsntggwsnytnccngtnacngt
ngayytnccnggngaygcngtngcnytnggnytncartgyttyacnytn
gayytnaaraaygtnacntgycartggcarcarcargaycaygcnwsnw
sncarggnttyttytaycaywsnmgngcnmgntgytgyccnmgngaymg
ntayccnathtgggaraaytgygargargargaraaracnaayccnggn
ytncaracnccncarttywsnmgntgycayttyaarwsnmgnaaygayw
snathathcayathytngtngargtnacnacngcnccnggnacngtnca
ywsntayytnggnwsnccnttytggathcaycargcngtnmgnytnccn
acnccnaayytncaytggmgngarathwsnwsnggncayytngarytng
artggcarcayccnwsnwsntgggcngcncargaracntgytaycaryt
nmgntayacnggngarggncaycargaytggaargtnytngarccnccn
ytnggngcnmgnggnggnacnytngarytnmgnccnmgnwsnmgntaym
gnytncarytnmgngcnmgnytnaayggnccnacntaycarggnccntg
gwsnwsntggwsngayccnacnmgngtngaracngcnacngaracngcn
tggathwsnytngtnacngcnytncayytngtnytnggnytnwsngcng
tnytnggnytnytnytnytnmgntggcarttyccngcncaytaymgnmg
nytnmgncaygcnytntggccnwsnytnccngayytncaymgngtnytn
ggncartayytnmgngayacngcngcnytnwsnccnccnaargcnacng
tnwsngayacntgygargargtngarccnwsnytnytngarathytncc
naarwsnwsngarmgnacnccnytnccnytntgywsnwsncargcncar
atggaytaymgnmgnytncarccnwsntgyytnggnacnatgccnytnw
sngtntgyccnccnatggcngarwsnggnwsntgytgyacnacncayat
hgcnaaycaywsntayytnccnytnwsntaytggcarcarccntrrtrr

SEQ ID NO: 2: TpoR.Δ60

580 amino acids presented in the N- to C-terminus direction, of which 1-491 (bold): TpoR extracellular domain, 492-513 (bold, underlined): TpoR TM domain, 514-580 (bold, italics): TpoR cytoplasmic domain with C-terminal truncation.

MPSWALFMVTSCLLLAPQNLAQVSSQDVSLLASDSEPLKCFSRTFEDLTC
FWDEEEAAPSGTYQLLYAYPREKPRACPLSSQSMPHFGTRYVCQFPDQEE
VRLFFPLHLWVKNVFLNQTRTQRVLFVDSVGLPAPPSIIKAMGGSQPGEL
QISWEEPAPEISDFLRYELRYGPRDPKNSTGPTVIQLIATETCCPALQRP
HSASALDQSPCAQPTMPWQDGPKQTSPSREASALTAEGGSCLISGLQPGN
SYWLQLRSEPDGISLGGSWGSWSLPVTVDLPGDAVALGLQCFTLDLKNVT
CQWQQQDHASSQGFFYHSRARCCPRDRYPIWENCEEEEKTNPGLQTPQFS
RCHFKSRNDSIIHILVEVTTAPGTVHSYLGSPFWIHQAVRLPTPNLHWRE
ISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHL
VLGLSAVLGLLLL [symbol] [symbol] [symbol] [symbol] [symbol] [symbol]
**
SEQ ID NO: 16: TpoR.Δ60
atgccnwsntgggcnytnttyatggtnacnwsntgyytnytnytngcncc
ncaraayytngcncargtnwsnwsncargaygtnwsnytnytngcnwsng
aywsngarccnytnaartgyttywsnmgnacnttygargayytnacntgy
ttytgggaygargargargcngcnccnwsnggnacntaycarytnytnta
ygcntayccnmgngaraarccnmgngcntgyccnytnwsnwsncarwsna
tgccncayttyggnacnmgntaygtntgycarttyccngaycargargar
gtnmgnytnttyttyccnytncayytntgggtnaaraaygtnttyytnaa
ycaracnmgnacncarmgngtnytnttygtngaywsngtnggnytnccng
cnccnccnwsnathathaargcnatgggnggnwsncarccnggngarytn
carathwsntgggargarccngcnccngarathwsngayttyytnmgnta
ygarytnmgntayggnccnmgngayccnaaraaywsnacnggnccnacng
tnathcarytnathgcnacngaracntgytgyccngcnytncarmgnccn
caywsngcnwsngcnytngaycarwsnccntgygcncarccnacnatgcc
ntggcargayggnccnaarcaracnwsnccnwsnmgngargcnwsngcny
tnacngcngarggnggnwsntgyytnathwsnggnytncarccnggnaay
wsntaytggytncarytnmgnwsngarccngayggnathwsnytnggngg
nwsntggggnwsntggwsnytnccngtnacngtngayytnccnggngayg
cngtngcnytnggnytncartgyttyacnytngayytnaaraaygtnacn
tgycartggcarcarcargaycaygcnwsnwsncarggnttyttytayca
ywsnmgngcnmgntgytgyccnmgngaymgntayccnathtgggaraayt
gygargargargaraaracnaayccnggnytncaracnccncarttywsn
mgntgycayttyaarwsnmgnaaygaywsnathathcayathytngtnga
rgtnacnacngcnccnggnacngtncaywsntayytnggnwsnccnttyt
ggathcaycargcngtnmgnytnccnacnccnaayytncaytggmgngar
athwsnwsnggncayytngarytngartggcarcayccnwsnwsntgggc
ngcncargaracntgytaycarytnmgntayacnggngarggncaycarg
aytggaargtnytngarccnccnytnggngcnmgnggnggnacnytngar
ytnmgnccnmgnwsnmgntaymgnytncarytnmgngcnmgnytnaaygg
nccnacntaycarggnccntggwsnwsntggwsngayccnacnmgngtng
aracngcnacngaracngcntggathwsnytngtnacngcnytncayytn
gtnytnggnytnwsngcngtnytnggnytnytnytnytnmgntggcartt
yccngcncaytaymgnmgnytnmgncaygcnytntggccnwsnytnccng
ayytncaymgngtnytnggncartayytnmgngayacngcngcnytnwsn
ccnccnaargcnacngtnwsngayacntgygargargtngarccnwsnyt
nytngarathytnccnaarwsnwsngarmgnacnccnytntrrtrr

SEQ ID NO: 3: TpoR-cyt.IL2rβ-cyt

626 amino acids presented in the N- to C-terminus direction, of which 1-491 (bold): TpoR extracellular domain, 492-513 (bold, underlined): TpoR TM domain, 514-538 (bold, italics): TpoR cytoplasmic domain with C-terminal truncation, 539-626 (unformatted): IL2rβ cytoplasmic domain.

MPSWALFMVTSCLLLAPQNLAQVSSQDVSLLASDSEPLKCFSRTFEDLTC
FWDEEEAAPSGTYQLLYAYPREKPRACPLSSQSMPHFGTRYVCQFPDQEE
VRLFFPLHLWVKNVFLNQTRTQRVLFVDSVGLPAPPSIIKAMGGSQPGEL
QISWEEPAPEISDFLRYELRYGPRDPKNSTGPTVIQLIATETCCPALQRP
HSASALDQSPCAQPTMPWQDGPKQTSPSREASALTAEGGSCLISGLQPGN
SYWLQLRSEPDGISLGGSWGSWSLPVTVDLPGDAVALGLQCFTLDLKNVT
CQWQQQDHASSQGFFYHSRARCCPRDRYPIWENCEEEEKTNPGLQTPQFS
RCHFKSRNDSIIHILVEVTTAPGTVHSYLGSPFWIHQAVRLPTPNLHWRE
ISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHL
VLGLSAVLGLLLL [symbol] [symbol] PRDWDPQPLGP
PTPGVPDLVDFQPPPELVLREAGEEVPDAGPREGVSFPWSRPPGQGEFRA
LNARLPLNTDAYLSLQELQGQDPTHLV**
SEQ ID NO: 17: TpoR.TpoR-cyt.IL2rβ-cyt
atgccnwsntgggcnytnttyatggtnacnwsntgyytnytnytngcncc
ncaraayytngcncargtnwsnwsncargaygtnwsnytnytngcnwsng
aywsngarccnytnaartgyttywsnmgnacnttygargayytnacntgy
ttytgggaygargargargcngcnccnwsnggnacntaycarytnytnta
ygcntayccnmgngaraarccnmgngcntgyccnytnwsnwsncarwsna
tgccncayttyggnacnmgntaygtntgycarttyccngaycargargar
gtnmgnytnttyttyccnytncayytntgggtnaaraaygtnttyytnaa
ycaracnmgnacncarmgngtnytnttygtngaywsngtnggnytnccng
cnccnccnwsnathathaargcnatgggnggnwsncarccnggngarytn
carathwsntgggargarccngcnccngarathwsngayttyytnmgnta
ygarytnmgntayggnccnmgngayccnaaraaywsnacnggnccnacng
tnathcarytnathgcnacngaracntgytgyccngcnytncarmgnccn
caywsngcnwsngcnytngaycarwsnccntgygcncarccnacnatgcc
ntggcargayggnccnaarcaracnwsnccnwsnmgngargcnwsngcny
tnacngcngarggnggnwsntgyytnathwsnggnytncarccnggnaay
wsntaytggytncarytnmgnwsngarccngayggnathwsnytnggngg
nwsntggggnwsntggwsnytnccngtnacngtngayytnccnggngayg
cngtngcnytnggnytncartgyttyacnytngayytnaaraaygtnacn
tgycartggcarcarcargaycaygcnwsnwsncarggnttyttytayca
ywsnmgngcnmgntgytgyccnmgngaymgntayccnathtgggaraayt
gygargargargaraaracnaayccnggnytncaracnccncarttywsn
mgntgycayttyaarwsnmgnaaygaywsnathathcayathytngtnga
rgtnacnacngcnccnggnacngtncaywsntayytnggnwsnccnttyt
ggathcaycargcngtnmgnytnccnacnccnaayytncaytggmgngar
athwsnwsnggncayytngarytngartggcarcayccnwsnwsntgggc
ngcncargaracntgytaycarytnmgntayacnggngarggncaycarg
aytggaargtnytngarccnccnytnggngcnmgnggnggnacnytngar
ytnmgnccnmgnwsnmgntaymgnytncarytnmgngcnmgnytnaaygg
nccnacntaycarggnccntggwsnwsntggwsngayccnacnmgngtng
aracngcnacngaracngcntggathwsnytngtnacngcnytncayytn
gtnytnggnytnwsngcngtnytnggnytnytnytnytnmgntggcartt
yccngcncaytaymgnmgnytnmgncaygcnytntggccnwsnytnccng
ayytncaymgngtnccnmgngaytgggayccncarccnytnggnccnccn
acnccnggngtnccngayytngtngayttycarccnccnccngarytngt
nytnmgngargcnggngargargtnccngaygcnggnccnmgngarggng
tnwsnttyccntggwsnmgnccnccnggncarggngarttymgngcnytn
aaygcnmgnytnccnytnaayacngaygcntayytnwsnytncargaryt
ncarggncargayccnacncayytngtntrrtrr

SEQ ID NO: 4: TpoR IL2rB-cyt.TpoR-cyt

808 amino acids presented in the N- to C-terminus direction, of which 1-491 (bold): TpoR extracellular domain, 492-513 (bold, underlined): TpoR TM domain, 514-709 (unformatted): IL2rB cytoplasmic domain, 710-808 (bold, italics): TpoR cytoplasmic domain with N-terminal truncation.

MPSWALFMVTSCLLLAPQNLAQVSSQDVSLLASDSEPLKCFSRTFEDLTC
FWDEEEAAPSGTYQLLYAYPREKPRACPLSSQSMPHFGTRYVCQFPDQEE
VRLFFPLHLWVKNVFLNQTRTQRVLFVDSVGLPAPPSIIKAMGGSQPGEL
QISWEEPAPEISDFLRYELRYGPRDPKNSTGPTVIQLIATETCCPALQRP
HSASALDQSPCAQPTMPWQDGPKQTSPSREASALTAEGGSCLISGLQPGN
SYWLQLRSEPDGISLGGSWGSWSLPVTVDLPGDAVALGLQCFTLDLKNVT
CQWQQQDHASSQGFFYHSRARCCPRDRYPIWENCEEEEKTNPGLQTPQFS
RCHFKSRNDSIIHILVEVTTAPGTVHSYLGSPFWIHQAVRLPTPNLHWRE
ISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHL
VLGLSAVLGLLLLNCRNTGPWLKKVLKCNTPDPSKFFSQLSSEHGGDVQK
WLSSPFPSSSFSPGGLAPEISPLEVLERDKVTQLLLQQDKVPEPASLSSN
HSLTSCFTNQGYFFFHLPDALEIEACQVYFTYDPYSEEDPDEGVAGAPTG
SSPQPLQPLSGEDDAYCTFPSRDDLLLFSPSLLGGPSPPSTAPGGSGAGE
ERMPPSLQE

[symbol]

[symbol]

[symbol]

[symbol]

[symbol]

[symbol]

[symbol]

[symbol]

[symbol]

**
SEQ ID NO: 18: TpoR.IL2rB-cyt.TpoR-cyt
atgccnwsntgggcnytnttyatggtnacnwsntgyytnytnytngcncc
ncaraayytngcncargtnwsnwsncargaygtnwsnytnytngcnwsng
aywsngarccnytnaartgyttywsnmgnacnttygargayytnacntgy
ttytgggaygargargargcngcnccnwsnggnacntaycarytnytnta
ygcntayccnmgngaraarccnmgngcntgyccnytnwsnwsncarwsna
tgccncayttyggnacnmgntaygtntgycarttyccngaycargargar
gtnmgnytnttyttyccnytncayytntgggtnaaraaygtnttyytnaa
ycaracnmgnacncarmgngtnytnttygtngaywsngtnggnytnccng
cnccnccnwsnathathaargcnatgggnggnwsncarccnggngarytn
carathwsntgggargarccngcnccngarathwsngayttyytnmgnta
ygarytnmgntayggnccnmgngayccnaaraaywsnacnggnccnacng
tnathcarytnathgcnacngaracntgytgyccngcnytncarmgnccn
caywsngcnwsngcnytngaycarwsnccntgygcncarccnacnatgcc
ntggcargayggnccnaarcaracnwsnccnwsnmgngargcnwsngcny
tnacngcngarggnggnwsntgyytnathwsnggnytncarccnggnaay
wsntaytggytncarytnmgnwsngarccngayggnathwsnytnggngg
nwsntggggnwsntggwsnytnccngtnacngtngayytnccnggngayg
cngtngcnytnggnytncartgyttyacnytngayytnaaraaygtnacn
tgycartggcarcarcargaycaygcnwsnwsncarggnttyttytayca
ywsnmgngcnmgntgytgyccnmgngaymgntayccnathtgggaraayt
gygargargargaraaracnaayccnggnytncaracnccncarttywsn
mgntgycayttyaarwsnmgnaaygaywsnathathcayathytngtnga
rgtnacnacngcnccnggnacngtncaywsntayytnggnwsnccnttyt
ggathcaycargcngtnmgnytnccnacnccnaayytncaytggmgngar
athwsnwsnggncayytngarytngartggcarcayccnwsnwsntgggc
ngcncargaracntgytaycarytnmgntayacnggngarggncaycarg
aytggaargtnytngarccnccnytnggngcnmgnggnggnacnytngar
ytnmgnccnmgnwsnmgntaymgnytncarytnmgngcnmgnytnaaygg
nccnacntaycarggnccntggwsnwsntggwsngayccnacnmgngtng
aracngcnacngaracngcntggathwsnytngtnacngcnytncayytn
gtnytnggnytnwsngcngtnytnggnytnytnytnytnaaytgymgnaa
yacnggnccntggytnaaraargtnytnaartgyaayacnccngayccnw
snaarttyttywsncarytnwsnwsngarcayggnggngaygtncaraar
tggytnwsnwsnccnttyccnwsnwsnwsnttywsnccnggnggnytngc
nccngarathwsnccnytngargtnytngarmgngayaargtnacncary
tnytnytncarcargayaargtnccngarccngcnwsnytnwsnwsnaay
caywsnytnacnwsntgyttyacnaaycarggntayttyttyttycayyt
nccngaygcnytngarathgargcntgycargtntayttyacntaygayc
cntaywsngargargayccngaygarggngtngcnggngcnccnacnggn
wsnwsnccncarccnytncarccnytnwsnggngargaygaygcntaytg
yacnttyccnwsnmgngaygayytnytnytnttywsnccnwsnytnytng
gnggnccnwsnccnccnwsnacngcnccnggnggnwsnggngcnggngar
garmgnatgccnccnwsnytncargarmgngtnytnggncartayytnmg
ngayacngcngcnytnwsnccnccnaargcnacngtnwsngayacntgyg
argargtngarccnwsnytnytngarathytnccnaarwsnwsngarmgn
acnccnytnccnytntgywsnwsncargcncaratggaytaymgnmgnyt
ncarccnwsntgyytnggnacnatgccnytnwsngtntgyccnccnatgg
cngarwsnggnwsntgytgyacnacncayathgcnaaycaywsntayytn
ccnytnwsntaytggcarcarccntrrtrr

SEQ ID NO: 5: TpoR SLAM

710 amino acids presented in the N- to C-terminus direction, of which 1-491 (bold): TpoR extracellular domain, 492-513 (bold, underlined): TpoR TM domain, 514-635 (bold, italics): TpoR cytoplasmic domain, 636-710 (unformatted): SLAM cytoplasmic domain.

MPSWALFMVTSCLLLAPQNLAQVSSQDVSLLASDSEPLKCFSRTFEDLTC
FWDEEEAAPSGTYQLLYAYPREKPRACPLSSQSMPHFGTRYVCQFPDQEE
VRLFFPLHLWVKNVFLNQTRTQRVLFVDSVGLPAPPSIIKAMGGSQPGEL
QISWEEPAPEISDFLRYELRYGPRDPKNSTGPTVIQLIATETCCPALQRP
HSASALDQSPCAQPTMPWQDGPKQTSPSREASALTAEGGSCLISGLQPGN
SYWLQLRSEPDGISLGGSWGSWSLPVTVDLPGDAVALGLQCFTLDLKNVT
CQWQQQDHASSQGFFYHSRARCCPRDRYPIWENCEEEEKTNPGLQTPQFS
RCHFKSRNDSIIHILVEVTTAPGTVHSYLGSPFWIHQAVRLPTPNLHWRE
ISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHL
VLGLSAVLGLLLL

[symbol]

[symbol]

[symbol]

[symbol]

[symbol]

[symbol]

[symbol]

[symbol]

[symbol]

[symbol]

RRRGKTNHYQTTVEKKSLTIYAQVQKPGPLQKKLDSFPAQDPCTTIYVAA
TEPVPESVQETNSITVYASVTLPES**
SEQ ID NO: 19: TpoR.SLAM
atgccnwsntgggcnytnttyatggtnacnwsntgyytnytnytngcncc
ncaraayytngcncargtnwsnwsncargaygtnwsnytnytngcnwsng
aywsngarccnytnaartgyttywsnmgnacnttygargayytnacntgy
ttytgggaygargargargcngcnccnwsnggnacntaycarytnytnta
ygcntayccnmgngaraarccnmgngcntgyccnytnwsnwsncarwsna
tgccncayttyggnacnmgntaygtntgycarttyccngaycargargar
gtnmgnytnttyttyccnytncayytntgggtnaaraaygtnttyytnaa
ycaracnmgnacncarmgngtnytnttygtngaywsngtnggnytnccng
cnccnccnwsnathathaargcnatgggnggnwsncarccnggngarytn
carathwsntgggargarccngcnccngarathwsngayttyytnmgnta
ygarytnmgntayggnccnmgngayccnaaraaywsnacnggnccnacng
tnathcarytnathgcnacngaracntgytgyccngcnytncarmgnccn
caywsngcnwsngcnytngaycarwsnccntgygcncarccnacnatgcc
ntggcargayggnccnaarcaracnwsnccnwsnmgngargcnwsngcny
tnacngcngarggnggnwsntgyytnathwsnggnytncarccnggnaay
wsntaytggytncarytnmgnwsngarccngayggnathwsnytnggngg
nwsntggggnwsntggwsnytnccngtnacngtngayytnccnggngayg
cngtngcnytnggnytncartgyttyacnytngayytnaaraaygtnacn
tgycartggcarcarcargaycaygcnwsnwsncarggnttyttytayca
ywsnmgngcnmgntgytgyccnmgngaymgntayccnathtgggaraayt
gygargargargaraaracnaayccnggnytncaracnccncarttywsn
mgntgycayttyaarwsnmgnaaygaywsnathathcayathytngtnga
rgtnacnacngcnccnggnacngtncaywsntayytnggnwsnccnttyt
ggathcaycargcngtnmgnytnccnacnccnaayytncaytggmgngar
athwsnwsnggncayytngarytngartggcarcayccnwsnwsntgggc
ngcncargaracntgytaycarytnmgntay acnggngarggncaycar
gaytggaargtnytngarccnccnytnggngcnmgnggnggnacnytnga
rytnmgnccnmgnwsnmgntaymgnytncarytnmgngcnmgnytnaayg
gnccnacntaycarggnccntggwsnwsntggwsngayccnacnmgngtn
garacngcnacngaracngcntggathwsnytngtnacngcnytncayyt
ngtnytnggnytnwsngcngtnytnggnytnytnytnytnmgntggcart
tyccngcncaytaymgnmgnytnmgncaygcnytntggccnwsnytnccn
gayytncaymgngtnytnggncartayytnmgngayacngcngcnytnws
nccnccnaargcnacngtnwsngayacntgygargargtngarccnwsny
tnytngarathytnccnaarwsnwsngarmgnacnccnytnccnytntgy
wsnwsncargcncaratggaytaymgnmgnytncarccnwsntgyytngg
nacnatgccnytnwsngtntgyccnccnatggcngarwsnggnwsntgyt
gyacnacncayathgcnaaycaywsntayytnccnytnwsntaytggcar
carccnmgnmgnmgnggnaaracnaaycaytaycaracnacngtngaraa
raarwsnytnacnathtaygcncargtncaraarccnggnccnytncara
araarytngaywsnttyccngcncargayccntgyacnacnathtaygtn
gcngcnacngarccngtnccngarwsngtncargaracnaaywsnathac
ngtntaygcnwsngtnacnytnccngarwsntrrtrr

SEQ ID NO: 6: TpoR.IL2rγ

721 amino acids presented in the N- to C-terminus direction, of which 1-491 (bold): TpoR extracellular domain, 492-513 (bold, underlined): TpoR TM domain, 514-635 (bold, italics): TpoR cytoplasmic domain, 636-721 (unformatted): IL2rγ cytoplasmic domain.

MPSWALFMVTSCLLLAPQNLAQVSSQDVSLLASDSEPLKCFSRTFEDLTC
FWDEEEAAPSGTYQLLYAYPREKPRACPLSSQSMPHFGTRYVCQFPDQEE
VRLFFPLHLWVKNVFLNQTRTQRVLFVDSVGLPAPPSIIKAMGGSQPGEL
QISWEEPAPEISDFLRYELRYGPRDPKNSTGPTVIQLIATETCCPALQRP
HSASALDQSPCAQPTMPWQDGPKQTSPSREASALTAEGGSCLISGLQPGN
SYWLQLRSEPDGISLGGSWGSWSLPVTVDLPGDAVALGLQCFTLDLKNVT
CQWQQQDHASSQGFFYHSRARCCPRDRYPIWENCEEEEKTNPGLQTPQFS
RCHFKSRNDSIIHILVEVTTAPGTVHSYLGSPFWIHQAVRLPTPNLHWRE
ISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHL
VLGLSAVLGLLLL

[symbol]

[symbol]

[symbol]

[symbol]

[symbol]

[symbol]

[symbol]

[symbol]

[symbol]

[symbol]

ERTMPRIPTLKNLEDLVTEYHGNFSAWSGVSKGLAESLQPDYSERLCLVS
EIPPKGGALGEGPGASPCNQHSPYWAPPCYTLKPET**
SEQ ID NO: 20: TpoR.IL2rγ
atgccnwsntgggcnytnttyatggtnacnwsntgyytnytnytngcncc
ncaraayytngcncargtnwsnwsncargaygtnwsnytnytngcnwsng
aywsngarccnytnaartgyttywsnmgnacnttygargayytnacntgy
ttytgggaygargargargcngcnccnwsnggnacntaycarytnytnta
ygcntayccnmgngaraarccnmgngcntgyccnytnwsnwsncarwsna
tgccncayttyggnacnmgntaygtntgycarttyccngaycargargar
gtnmgnytnttyttyccnytncayytntgggtnaaraaygtnttyytnaa
ycaracnmgnacncarmgngtnytnttygtngaywsngtnggnytnccng
cnccnccnwsnathathaargcnatgggnggnwsncarccnggngarytn
carathwsntgggargarccngcnccngarathwsngayttyytnmgnta
ygarytnmgntayggnccnmgngayccnaaraaywsnacnggnccnacng
tnathcarytnathgcnacngaracntgytgyccngcnytncarmgnccn
caywsngcnwsngcnytngaycarwsnccntgygcncarccnacnatgcc
ntggcargayggnccnaarcaracnwsnccnwsnmgngargcnwsngcny
tnacngcngarggnggnwsntgyytnathwsnggnytncarccnggnaay
wsntaytggytncarytnmgnwsngarccngayggnathwsnytnggngg
nwsntggggnwsntggwsnytnccngtnacngtngayytnccnggngayg
cngtngcnytnggnytncartgyttyacnytngayytnaaraaygtnacn
tgycartggcarcarcargaycaygcnwsnwsncarggnttyttytayca
ywsnmgngcnmgntgytgyccnmgngaymgntayccnathtgggaraayt
gygargargargaraaracnaayccnggnytncaracnccncarttywsn
mgntgycayttyaarwsnmgnaaygaywsnathathcayathytngtnga
rgtnacnacngcnccnggnacngtncaywsntayytnggnwsnccnttyt
ggathcaycargcngtnmgnytnccnacnccnaayytncaytggmgngar
athwsnwsnggncayytngarytngartggcarcayccnwsnwsntgggc
ngcncargaracntgytaycarytnmgntayacnggngarggncaycarg
aytggaargtnytngarccnccnytnggngcnmgnggnggnacnytngar
ytnmgnccnmgnwsnmgntaymgnytncarytnmgngcnmgnytnaaygg
nccnacntaycarggnccntggwsnwsntggwsngayccnacnmgngtng
aracngcnacngaracngcntggathwsnytngtnacngcnytncayytn
gtnytnggnytnwsngcngtnytnggnytnytnytnytnmgntggcartt
yccngcncaytaymgnmgnytnmgncaygcnytntggccnwsnytnccng
ayytncaymgngtnytnggncartayytnmgngayacngcngcnytnwsn
ccnccnaargcnacngtnwsngayacntgygargargtngarccnwsnyt
nytngarathytnccnaarwsnwsngarmgnacnccnytnccnytntgyw
snwsncargcncaratggaytaymgnmgnytncarccnwsntgyytnggn
acnatgccnytnwsngtntgyccnccnatggcngarwsnggnwsntgytg
yacnacncayathgcnaaycaywsntayytnccnytnwsntaytggcarc
arccngarmgnacnatgccnmgnathccnacnytnaaraayytngargay
ytngtnacngartaycayggnaayttywsngcntggwsnggngtnwsnaa
rggnytngcngarwsnytncarccngaytaywsngarmgnytntgyytng
tnwsngarathccnccnaarggnggngcnytnggngarggnccnggngcn
wsnccntgyaaycarcaywsnccntaytgggcnccnccntgytayacnyt
naarccngaracntrrtrr

SEQ ID NO: 7: TpoR-TLR1

817 amino acids presented in the N- to C-terminus direction, of which 1-491 (bold): TpoR extracellular domain, 492-513 (bold, underlined): TpoR TM domain, 514-635 (bold, italics): TpoR cytoplasmic domain, 636-817 (unformatted): TLR1 cytoplasmic domain.

MPSWALFMVTSCLLLAPQNLAQVSSQDVSLLASDSEPLKCFSRTFEDLTC
FWDEEEAAPSGTYQLLYAYPREKPRACPLSSQSMPHFGTRYVCQFPDQEE
VRLFFPLHLWVKNVFLNQTRTQRVLFVDSVGLPAPPSIIKAMGGSQPGEL
QISWEEPAPEISDFLRYELRYGPRDPKNSTGPTVIQLIATETCCPALQRP
HSASALDQSPCAQPTMPWQDGPKQTSPSREASALTAEGGSCLISGLQPGN
SYWLQLRSEPDGISLGGSWGSWSLPVTVDLPGDAVALGLQCFTLDLKNVT
CQWQQQDHASSQGFFYHSRARCCPRDRYPIWENCEEEEKTNPGLQTPQFS
RCHFKSRNDSIIHILVEVTTAPGTVHSYLGSPFWIHQAVRLPTPNLHWRE
ISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHL
VLGLSAVLGLLLL

[symbol]

[symbol]

[symbol]

[symbol]

[symbol]

[symbol]

[symbol]

[symbol]

[symbol]

[symbol]

DLPWYLRMVCQWTQTRRRARNIPLEELQRNLQFH
AFISYSGHDSFWVKNELLPNLEKEGMQICLHERNFVPGKSIVENIITCI
EKSYKSIFVLSPNFVQSEWCHYELYFAHHNLFHEGSNSLILILLEPIPQ
YSIPSSYEIKLKSLMARRTYLEWPKEKSKRGLFWANLRAAINIKLTEQA
KK**
SEQ ID NO: 21: TpoR-TLR1
atgccnwsntgggcnytnttyatggtnacnwsntgyytnytnytngcncc
ncaraayytngcncargtnwsnwsncargaygtnwsnytnytngcnwsng
aywsngarccnytnaartgyttywsnmgnacnttygargayytnacntgy
ttytgggaygargargargcngcnccnwsnggnacntaycarytnytnta
ygcntayccnmgngaraarccnmgngcntgyccnytnwsnwsncarwsna
tgccncayttyggnacnmgntaygtntgycarttyccngaycargargar
gtnmgnytnttyttyccnytncayytntgggtnaaraaygtnttyytnaa
ycaracnmgnacncarmgngtnytnttygtngaywsngtnggnytnccng
cnccnccnwsnathathaargcnatgggnggnwsncarccnggngarytn
carathwsntgggargarccngcnccngarathwsngayttyytnmgnta
ygarytnmgntayggnccnmgngayccnaaraaywsnacnggnccnacng
tnathcarytnathgcnacngaracntgytgyccngcnytncarmgnccn
caywsngcnwsngcnytngaycarwsnccntgygcncarccnacnatgcc
ntggcargayggnccnaarcaracnwsnccnwsnmgngargcnwsngcny
tnacngcngarggnggnwsntgyytnathwsnggnytncarccnggnaay
wsntaytggytncarytnmgnwsngarccngayggnathwsnytnggngg
nwsntggggnwsntggwsnytnccngtnacngtngayytnccnggngayg
cngtngcnytnggnytncartgyttyacnytngayytnaaraaygtnacn
tgycartggcarcarcargaycaygcnwsnwsncarggnttyttytayca
ywsnmgngcnmgntgytgyccnmgngaymgntayccnathtgggaraayt
gygargargargaraaracnaayccnggnytncaracnccncarttywsn
mgntgycayttyaarwsnmgnaaygaywsnathathcayathytngtnga
rgtnacnacngcnccnggnacngtncaywsntayytnggnwsnccnttyt
ggathcaycargcngtnmgnytnccnacnccnaayytncaytggmgngar
athwsnwsnggncayytngarytngartggcarcayccnwsnwsntgggc
ngcncargaracntgytaycarytnmgntayacnggngarggncaycarg
aytggaargtnytngarccnccnytnggngcnmgnggnggnacnytngar
ytnmgnccnmgnwsnmgntaymgnytncarytnmgngcnmgnytnaaygg
nccnacntaycarggnccntggwsnwsntggwsngayccnacnmgngtng
aracngcnacngaracngcntggathwsnytngtnacngcnytncayytn
gtnytnggnytnwsngcngtnytnggnytnytnytnytnmgntggcartt
yccngcncaytaymgnmgnytnmgncaygcnytntggccnwsnytnccng
ayytncaymgngtnytnggncartayytnmgngayacngcngcnytnwsn
ccnccnaargcnacngtnwsngayacntgygargargtngarccnwsnyt
nytngarathytnccnaarwsnwsngarmgnacnccnytnccnytntgyw
snwsncargcncaratggaytaymgnmgnytncarccnwsntgyytnggn
acnatgccnytnwsngtntgyccnccnatggcngarwsnggnwsntgytg
yacnacncayathgcnaaycaywsntayytnccnytnwsntaytggcarc
arccngayytnccntggtayytnmgnatggtntgycartggacncaracn
mgnmgnmgngcnmgnaayathccnytngargarytncarmgnaayytnca
rttycaygcnttyathwsntaywsnggncaygaywsnttytgggtnaara
aygarytnytnccnaayytngaraargarggnatgcarathtgyytncay
garmgnaayttygtnccnggnaarwsnathgtngaraayathathacntg
yathgaraarwsntayaarwsnathttygtnytnwsnccnaayttygtnc
arwsngartggtgycaytaygarytntayttygcncaycayaayytntty
caygarggnwsnaaywsnytnathytnathytnytngarccnathccnca
rtaywsnathccnwsnwsntaycayaarytnaarwsnytnatggcnmgnm
gnacntayytngartggccnaargaraarwsnaarmgnggnytnttytgg
gcnaayytnmgngcngcnathaayathaarytnacngarcargcnaaraa
rtrrtrr

SEQ ID NO: 8: TpoR-TIAF1

750 amino acids presented in the N- to C-terminus direction, of which 1-491 (bold): TpoR extracellular domain, 492-513 (bold, underlined): TpoR TM domain, 514-635 (bold, italics): TpoR cytoplasmic domain, 636-750 (unformatted): TIAF1 cytoplasmic domain.

MPSWALFMVTSCLLLAPQNLAQVSSQDVSLLASDSEPLKCFSRTFEDLTC
FWDEEEAAPSGTYQLLYAYPREKPRACPLSSQSMPHFGTRYVCQFPDQEE
VRLFFPLHLWVKNVFLNQTRTQRVLFVDSVGLPAPPSIIKAMGGSQPGEL
QISWEEPAPEISDFLRYELRYGPRDPKNSTGPTVIQLIATETCCPALQRP
HSASALDQSPCAQPTMPWQDGPKQTSPSREASALTAEGGSCLISGLQPGN
SYWLQLRSEPDGISLGGSWGSWSLPVTVDLPGDAVALGLQCFTLDLKNVT
CQWQQQDHASSQGFFYHSRARCCPRDRYPIWENCEEEEKTNPGLQTPQFS
RCHFKSRNDSIIHILVEVTTAPGTVHSYLGSPFWIHQAVRLPTPNLHWRE
ISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHL
VLGLSAVLGLLLL

[symbol]

[symbol]

[symbol]

[symbol]

[symbol]

[symbol]

[symbol]

[symbol]

[symbol]

[symbol]

MSSPSSPFREQ
SFLCAAGDAGEESRVQVLKNEVRRGSPVLLGWVEQAYADKCVCGPSAPPA
PTPPSLSQRVMCNDLFKVNPFQLQQFRADPSTASLLLCPGGLDHKLNLRG
KAWG**
SEQ ID NO: 22: TpoR-TIAF1
atgccnwsntgggcnytnttyatggtnacnwsntgyytnytnytngcncc
ncaraayytngcncargtnwsnwsncargaygtnwsnytnytngcnwsng
aywsngarccnytnaartgyttywsnmgnacnttygargayytnacntgy
ttytgggaygargargargcngcnccnwsnggnacntaycarytnytnta
ygcntayccnmgngaraarccnmgngcntgyccnytnwsnwsncarwsna
tgccncayttyggnacnmgntaygtntgycarttyccngaycargargar
gtnmgnytnttyttyccnytncayytntgggtnaaraaygtnttyytnaa
ycaracnmgnacncarmgngtnytnttygtngaywsngtnggnytnccng
cnccnccnwsnathathaargcnatgggnggnwsncarccnggngarytn
carathwsntgggargarccngcnccngarathwsngayttyytnmgnta
ygarytnmgntayggnccnmgngayccnaaraaywsnacnggnccnacng
tnathcarytnathgcnacngaracntgytgyccngcnytncarmgnccn
caywsngcnwsngcnytngaycarwsnccntgygcncarccnacnatgcc
ntggcargayggnccnaarcaracnwsnccnwsnmgngargcnwsngcny
tnacngcngarggnggnwsntgyytnathwsnggnytncarccnggnaay
wsntaytggytncarytnmgnwsngarccngayggnathwsnytnggngg
nwsntggggnwsntggwsnytnccngtnacngtngayytnccnggngayg
cngtngcnytnggnytncartgyttyacnytngayytnaaraaygtnacn
tgycartggcarcarcargaycaygcnwsnwsncarggnttyttytayca
ywsnmgngcnmgntgytgyccnmgngaymgntayccnathtgggaraayt
gygargargargaraaracnaayccnggnytncaracnccncarttywsn
mgntgycayttyaarwsnmgnaaygaywsnathathcayathytngtnga
rgtnacnacngcnccnggnacngtncaywsntayytnggnwsnccnttyt
ggathcaycargcngtnmgnytnccnacnccnaayytncaytggmgngar
athwsnwsnggncayytngarytngartggcarcayccnwsnwsntgggc
ngcncargaracntgytaycarytnmgntayacnggngarggncaycarg
aytggaargtnytngarccnccnytnggngcnmgnggnggnacnytngar
ytnmgnccnmgnwsnmgntaymgnytncarytnmgngcnmgnytnaaygg
nccnacntaycarggnccntggwsnwsntggwsngayccnacnmgngtng
aracngcnacngaracngcntggathwsnytngtnacngcnytncayytn
gtnytnggnytnwsngcngtnytnggnytnytnytnytnmgntggcartt
yccngcncaytaymgnmgnytnmgncaygcnytntggccnwsnytnccng
ayytncaymgngtnytnggncartayytnmgngayacngcngcnytnwsn
ccnccnaargcnacngtnwsngayacntgygargargtngarccnwsnyt
nytngarathytnccnaarwsnwsngarmgnacnccnytnccnytntgyw
snwsncargcncaratggaytaymgnmgnytncarccnwsntgyytnggn
acnatgccnytnwsngtntgyccnccnatggcngarwsnggnwsntgytg
yacnacncayathgcnaaycaywsntayytnccnytnwsntaytggcarc
arccnatgwsnwsnccnwsnwsnccnttymgngarcarwsnttyytntgy
gcngcnggngaygcnggngargarwsnmgngtncargtnytnaaraayga
rgtnmgnmgnggnwsnccngtnytnytnggntgggtngarcargcntayg
cngayaartgygtntgyggnccnwsngcnccnccngcnccnacnccnccn
wsnytnwsncarmgngtnatgtgyaaygayytnttyaargtnaayccntt
ycarytncarcarttymgngcngayccnwsnacngcnwsnytnytnytnt
gyccnggnggnytngaycayaarytnaayytnmgnggnaargcntggggn
trrtrr

SEQ ID NO: 9: TpoR-CD40

697 amino acids presented in the N- to C-terminus direction, of which 1-491 (bold): TpoR extracellular domain, 492-513 (bold, underlined): TpoR TM domain, 514-635 (bold, italics): TpoR cytoplasmic domain, 636-697 (unformatted): CD40 cytoplasmic domain.

MPSWALFMVTSCLLLAPQNLAQVSSQDVSLLASDSEPLKCFSRTFEDLTC
FWDEEEAAPSGTYQLLYAYPREKPRACPLSSQSMPHFGTRYVCQFPDQEE
VRLFFPLHLWVKNVFLNQTRTQRVLFVDSVGLPAPPSIIKAMGGSQPGEL
QISWEEPAPEISDFLRYELRYGPRDPKNSTGPTVIQLIATETCCPALQRP
HSASALDQSPCAQPTMPWQDGPKQTSPSREASALTAEGGSCLISGLQPGN
SYWLQLRSEPDGISLGGSWGSWSLPVTVDLPGDAVALGLQCFTLDLKNVT
CQWQQQDHASSQGFFYHSRARCCPRDRYPIWENCEEEEKTNPGLQTPQFS
RCHFKSRNDSIIHILVEVTTAPGTVHSYLGSPFWIHQAVRLPTPNLHWRE
ISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHL
VLGLSAVLGLLLL

[symbol]

[symbol]

[symbol]

[symbol]

[symbol]

[symbol]

[symbol]

[symbol]

[symbol]

[symbol]

KKVAKKPTNKAPHPKQEPQEINFPDDLPGSNTAAPVQETLHGCQPVTQED
GKESRISVQERQ**
SEQ ID NO: 23: TpoR-CD40
atgccnwsntgggcnytnttyatggtnacnwsntgyytnytnytngcncc
ncaraayytngcncargtnwsnwsncargaygtnwsnytnytngcnwsng
aywsngarccnytnaartgyttywsnmgnacnttygargayytnacntgy
ttytgggaygargargargcngcnccnwsnggnacntaycarytnytnta
ygcntayccnmgngaraarccnmgngcntgyccnytnwsnwsncarwsna
tgccncayttyggnacnmgntaygtntgycarttyccngaycargargar
gtnmgnytnttyttyccnytncayytntgggtnaaraaygtnttyytnaa
ycaracnmgnacncarmgngtnytnttygtngaywsngtnggnytnccng
cnccnccnwsnathathaargcnatgggnggnwsncarccnggngarytn
carathwsntgggargarccngcnccngarathwsngayttyytnmgnta
ygarytnmgntayggnccnmgngayccnaaraaywsnacnggnccnacng
tnathcarytnathgcnacngaracntgytgyccngcnytncarmgnccn
caywsngcnwsngcnytngaycarwsnccntgygcncarccnacnatgcc
ntggcargayggnccnaarcaracnwsnccnwsnmgngargcnwsngcny
tnacngcngarggnggnwsntgyytnathwsnggnytncarccnggnaay
wsntaytggytncarytnmgnwsngarccngayggnathwsnytnggngg
nwsntggggnwsntggwsnytnccngtnacngtngayytnccnggngayg
cngtngcnytnggnytncartgyttyacnytngayytnaaraaygtnacn
tgycartggcarcarcargaycaygcnwsnwsncarggnttyttytayca
ywsnmgngcnmgntgytgyccnmgngaymgntayccnathtgggaraayt
gygargargargaraaracnaayccnggnytncaracnccncarttywsn
mgntgycayttyaarwsnmgnaaygaywsnathathcayathytngtnga
rgtnacnacngcnccnggnacngtncaywsntayytnggnwsnccnttyt
ggathcaycargcngtnmgnytnccnacnccnaayytncaytggmgngar
athwsnwsnggncayytngarytngartggcarcayccnwsnwsntgggc
ngcncargaracntgytaycarytnmgntayacnggngarggncaycarg
aytggaargtnytngarccnccnytnggngcnmgnggnggnacnytngar
ytnmgnccnmgnwsnmgntaymgnytncarytnmgngcnmgnytnaaygg
nccnacntaycarggnccntggwsnwsntggwsngayccnacnmgngtng
aracngcnacngaracngcntggathwsnytngtnacngcnytncayytn
gtnytnggnytnwsngcngtnytnggnytnytnytnytnmgntggcartt
yccngcncaytaymgnmgnytnmgncaygcnytntggccnwsnytnccng
ayytncaymgngtnytnggncartayytnmgngayacngcngcnytnwsn
ccnccnaargcnacngtnwsngayacntgygargargtngarccnwsnyt
nytngarathytnccnaarwsnwsngarmgnacnccnytnccnytntgyw
snwsncargcncaratggaytaymgnmgnytncarccnwsntgyytnggn
acnatgccnytnwsngtntgyccnccnatggcngarwsnggnwsntgytg
yacnacncayathgcnaaycaywsntayytnccnytnwsntaytggcarc
arccnaaraargtngcnaaraarccnacnaayaargcnccncayccnaar
cargarccncargarathaayttyccngaygayytnccnggnwsnaayac
ngcngcnccngtncargaracnytncayggntgycarccngtnacncarg
argayggnaargarwsnmgnathwsngtncargarmgncartrrtrr

SEQ ID NO: 10: TpoR-ITAM1

676 amino acids presented in the N- to C-terminus direction, of which 1-491 (bold): TpoR extracellular domain, 492-513 (bold, underlined): TpoR TM domain, 514-635 (bold, italics): TpoR cytoplasmic domain, 636-676 (unformatted): ITAM1 cytoplasmic domain.

MPSWALFMVTSCLLLAPQNLAQVSSQDVSLLASDSEPLKCFSRTFEDLTC
FWDEEEAAPSGTYQLLYAYPREKPRACPLSSQSMPHFGTRYVCQFPDQEE
VRLFFPLHLWVKNVFLNQTRTQRVLFVDSVGLPAPPSIIKAMGGSQPGEL
QISWEEPAPEISDFLRYELRYGPRDPKNSTGPTVIQLIATETCCPALQRP
HSASALDQSPCAQPTMPWQDGPKQTSPSREASALTAEGGSCLISGLQPGN
SYWLQLRSEPDGISLGGSWGSWSLPVTVDLPGDAVALGLQCFTLDLKNVT
CQWQQQDHASSQGFFYHSRARCCPRDRYPIWENCEEEEKTNPGLQTPQFS
RCHFKSRNDSIIHILVEVTTAPGTVHSYLGSPFWIHQAVRLPTPNLHWRE
ISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHL
VLGLSAVLGLLLL

[symbol]

[symbol]

[symbol]

[symbol]

[symbol]

[symbol]

[symbol]

[symbol]

[symbol]

[symbol]

RVKFSRSADAPAYQQGQNQLYNELNLGRREEYDVLDKRRGR**
SEQ ID NO: 24: TpoR-ITAM1
atgccnwsntgggcnytnttyatggtnacnwsntgyytnytnytngcncc
ncaraayytngcncargtnwsnwsncargaygtnwsnytnytngcnwsng
aywsngarccnytnaartgyttywsnmgnacnttygargayytnacntgy
ttytgggaygargargargcngcnccnwsnggnacntaycarytnytnta
ygcntayccnmgngaraarccnmgngcntgyccnytnwsnwsncarwsna
tgccncayttyggnacnmgntaygtntgycarttyccngaycargargar
gtnmgnytnttyttyccnytncayytntgggtnaaraaygtnttyytnaa
ycaracnmgnacncarmgngtnytnttygtngaywsngtnggnytnccng
cnccnccnwsnathathaargcnatgggnggnwsncarccnggngarytn
carathwsntgggargarccngcnccngarathwsngayttyytnmgnta
ygarytnmgntayggnccnmgngayccnaaraaywsnacnggnccnacng
tnathcarytnathgcnacngaracntgytgyccngcnytncarmgnccn
caywsngcnwsngcnytngaycarwsnccntgygcncarccnacnatgcc
ntggcargayggnccnaarcaracnwsnccnwsnmgngargcnwsngcny
tnacngcngarggnggnwsntgyytnathwsnggnytncarccnggnaay
wsntaytggytncarytnmgnwsngarccngayggnathwsnytnggngg
nwsntggggnwsntggwsnytnccngtnacngtngayytnccnggngayg
cngtngcnytnggnytncartgyttyacnytngayytnaaraaygtnacn
tgycartggcarcarcargaycaygcnwsnwsncarggnttyttytayca
ywsnmgngcnmgntgytgyccnmgngaymgntayccnathtgggaraayt
gygargargargaraaracnaayccnggnytncaracnccncarttywsn
mgntgycayttyaarwsnmgnaaygaywsnathathcayathytngtnga
rgtnacnacngcnccnggnacngtncaywsntayytnggnwsnccnttyt
ggathcaycargcngtnmgnytnccnacnccnaayytncaytggmgngar
athwsnwsnggncayytngarytngartggcarcayccnwsnwsntgggc
ngcncargaracntgytaycarytnmgntayacnggngarggncaycarg
aytggaargtnytngarccnccnytnggngcnmgnggnggnacnytngar
ytnmgnccnmgnwsnmgntaymgnytncarytnmgngcnmgnytnaaygg
nccnacntaycarggnccntggwsnwsntggwsngayccnacnmgngtng
aracngcnacngaracngcntggathwsnytngtnacngcnytncayytn
gtnytnggnytnwsngcngtnytnggnytnytnytnytnmgntggcartt
yccngcncaytaymgnmgnytnmgncaygcnytntggccnwsnytnccng
ayytncaymgngtnytnggncartayytnmgngayacngcngcnytnwsn
ccnccnaargcnacngtnwsngayacntgygargargtngarccnwsnyt
nytngarathytnccnaarwsnwsngarmgnacnccnytnccnytntgyw
snwsncargcncaratggaytaymgnmgnytncarccnwsntgyytnggn
acnatgccnytnwsngtntgyccnccnatggcngarwsnggnwsntgytg
yacnacncayathgcnaaycaywsntayytnccnytnwsntaytggcarc
arccnmgngtnaarttywsnmgnwsngcngaygcnccngcntaycarcar
ggncaraaycarytntayaaygarytnaayytnggnmgnmgngargarta
ygaygtnytngayaarmgnmgnggnmgntrrtrr

SE0 ID NO: 11: TpoR TpoR-cyt LMP1-cyt

836 amino acids presented in the N- to C-terminus direction, of which 1-491 (bold): TpoR extracellular domain, 492-513 (bold, underlined): TpoR TM domain, 514-635 (bold, italics): TpoR cytoplasmic domain, 636-836 (unformatted): LMP-1 cytoplasmic domain.

MPSWALFMVTSCLLLAPQNLAQVSSQDVSLLASDSEPLKCFSRTFEDLTC
FWDEEEAAPSGTYQLLYAYPREKPRACPLSSQSMPHFGTRYVCQFPDQEE
VRLFFPLHLWVKNVFLNQTRTQRVLFVDSVGLPAPPSIIKAMGGSQPGEL
QISWEEPAPEISDFLRYELRYGPRDPKNSTGPTVIQLIATETCCPALQRP
HSASALDQSPCAQPTMPWQDGPKQTSPSREASALTAEGGSCLISGLQPGN
SYWLQLRSEPDGISLGGSWGSWSLPVTVDLPGDAVALGLQCFTLDLKNVT
CQWQQQDHASSQGFFYHSRARCCPRDRYPIWENCEEEEKTNPGLQTPQFS
RCHFKSRNDSIIHILVEVTTAPGTVHSYLGSPFWIHQAVRLPTPNLHWRE
ISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHL
VLGLSAVLGLLLL

[symbol]

[symbol]

[symbol]

[symbol]

[symbol]

[symbol]

[symbol]

[symbol]

[symbol]

[symbol]

YHGQRHSDEHHHDDSLPHPQQATDDSGHESDSNSNEGRHEILLVSGAGDG
PPLCSQNLGAPGGGPDNGPQDPDNTDDNGPQDPDNTDDNGPHDPLPQDPD
NTDDNGPQDPDNTDDNGPHDPLPHSPSDSAGNDGGPPQLTEEVENKGGDQ
GPPLMTDGGGGHSHDSGHGGGDPHLPTLLLGSSGSGGDDDDPHGPVQLSY
YD**
SEQ ID NO: 25: TpoR.TpoR-cyt.LMP1-cyt
atgccnwsntgggcnytnttyatggtnacnwsntgyytnytnytngcncc
ncaraayytngcncargtnwsnwsncargaygtnwsnytnytngcnwsng
aywsngarccnytnaartgyttywsnmgnacnttygargayytnacntgy
ttytgggaygargargargcngcnccnwsnggnacntaycarytnytnta
ygcntayccnmgngaraarccnmgngcntgyccnytnwsnwsncarwsna
tgccncayttyggnacnmgntaygtntgycarttyccngaycargargar
gtnmgnytnttyttyccnytncayytntgggtnaaraaygtnttyytnaa
ycaracnmgnacncarmgngtnytnttygtngaywsngtnggnytnccng
cnccnccnwsnathathaargcnatgggnggnwsncarccnggngarytn
carathwsntgggargarccngcnccngarathwsngayttyytnmgnta
ygarytnmgntayggnccnmgngayccnaaraaywsnacnggnccnacng
tnathcarytnathgcnacngaracntgytgyccngcnytncarmgnccn
caywsngcnwsngcnytngaycarwsnccntgygcncarccnacnatgcc
ntggcargayggnccnaarcaracnwsnccnwsnmgngargcnwsngcny
tnacngcngarggnggnwsntgyytnathwsnggnytncarccnggnaay
wsntaytggytncarytnmgnwsngarccngayggnathwsnytnggngg
nwsntggggnwsntggwsnytnccngtnacngtngayytnccnggngayg
cngtngcnytnggnytncartgyttyacnytngayytnaaraaygtnacn
tgycartggcarcarcargaycaygcnwsnwsncarggnttyttytayca
ywsnmgngcnmgntgytgyccnmgngaymgntayccnathtgggaraayt
gygargargargaraaracnaayccnggnytncaracnccncarttywsn
mgntgycayttyaarwsnmgnaaygaywsnathathcayathytngtnga
rgtnacnacngcnccnggnacngtncaywsntayytnggnwsnccnttyt
ggathcaycargcngtnmgnytnccnacnccnaayytncaytggmgngar
athwsnwsnggncayytngarytngartggcarcayccnwsnwsntgggc
ngcncargaracntgytaycarytnmgntayacnggngarggncaycarg
aytggaargtnytngarccnccnytnggngcnmgnggnggnacnytngar
ytnmgnccnmgnwsnmgntaymgnytncarytnmgngcnmgnytnaaygg
nccnacntaycarggnccntggwsnwsntggwsngayccnacnmgngtng
aracngcnacngaracngcntggathwsnytngtnacngcnytncayytn
gtnytnggnytnwsngcngtnytnggnytnytnytnytnmgntggcartt
yccngcncaytaymgnmgnytnmgncaygcnytntggccnwsnytnccng
ayytncaymgngtnytnggncartayytnmgngayacngcngcnytnwsn
ccnccnaargcnacngtnwsngayacntgygargargtngarccnwsnyt
nytngarathytnccnaarwsnwsngarmgnacnccnytnccnytntgyw
snwsncargcncaratggaytaymgnmgnytncarccnwsntgyytnggn
acnatgccnytnwsngtntgyccnccnatggcngarwsnggnwsntgytg
yacnacncayathgcnaaycaywsntayytnccnytnwsntaytggcarc
arccntaycayggncarmgncaywsngaygarcaycaycaygaygaywsn
ytnccncayccncarcargcnacngaygaywsnggncaygarwsngayws
naaywsnaaygarggnmgncaycayytnytngtnwsnggngcnggngayg
gnccnccnytntgywsncaraayytnggngcnccnggnggnggnccngay
aayggnccncargayccngayaayacngaygayaayggnccncargaycc
ngayaayacngaygayaayggnccncaygayccnytnccncargayccng
ayaayacngaygayaayggnccncargayccngayaayacngaygayaay
ggnccncaygayccnytnccncaywsnccnwsngaywsngcnggnaayga
yggnggnccnccncarytnacngargargtngaraayaarggnggngayc
arggnccnccnytnatgacngayggnggnggnggncaywsncaygaywsn
ggncayggnggnggngayccncayytnccnacnytnytnytnggnwsnws
nggnwsnggnggngaygaygaygayccncayggnccngtncarytnwsnt
aytaygaytrrtrr

SEQ ID NO: 12: TpoR LMP1-cyt

714 amino acids presented in the N- to C-terminus direction, of which 1-491 (bold): TpoR extracellular domain, 492-513 (bold, underlined): TpoR TM domain, 514-714 (unformatted): LMP-1 cytoplasmic domain.

MPSWALFMVTSCLLLAPQNLAQVSSQDVSLLASDSEPLKCFSRTFEDLTC
FWDEEEAAPSGTYQLLYAYPREKPRACPLSSQSMPHFGTRYVCQFPDQEE
VRLFFPLHLWVKNVFLNQTRTQRVLFVDSVGLPAPPSIIKAMGGSQPGEL
QISWEEPAPEISDFLRYELRYGPRDPKNSTGPTVIQLIATETCCPALQRP
HSASALDQSPCAQPTMPWQDGPKQTSPSREASALTAEGGSCLISGLQPGN
SYWLQLRSEPDGISLGGSWGSWSLPVTVDLPGDAVALGLQCFTLDLKNVT
CQWQQQDHASSQGFFYHSRARCCPRDRYPIWENCEEEEKTNPGLQTPQFS
RCHFKSRNDSIIHILVEVTTAPGTVHSYLGSPFWIHQAVRLPTPNLHWRE
ISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHL
VLGLSAVLGLLLLYHGQRHSDEHHHDDSLPHPQQATDDSGHESDSNSNEG
RHHLLVSGAGDGPPLCSQNLGAPGGGPDNGPQDPDNTDDNGPQDPDNTDD
NGPHDPLPQDPDNTDDNGPQDPDNTDDNGPHDPLPHSPSDSAGNDGGPPQ
LTEEVENKGGDQGPPLMTDGGGGHSHDSGHGGGDPHLPTLLLGSSGSGGD
DDDPHGPVQLSYYD**
SEQ ID NO: 26: TpoR.LMP1-cyt
atgccnwsntgggcnytnttyatggtnacnwsntgyytnytnytngcncc
ncaraayytngcncargtnwsnwsncargaygtnwsnytnytngcnwsng
aywsngarccnytnaartgyttywsnmgnacnttygargayytnacntgy
ttytgggaygargargargcngcnccnwsnggnacntaycarytnytnta
ygcntayccnmgngaraarccnmgngcntgyccnytnwsnwsncarwsna
tgccncayttyggnacnmgntaygtntgycarttyccngaycargargar
gtnmgnytnttyttyccnytncayytntgggtnaaraaygtnttyytnaa
ycaracnmgnacncarmgngtnytnttygtngaywsngtnggnytnccng
cnccnccnwsnathathaargcnatgggnggnwsncarccnggngarytn
carathwsntgggargarccngcnccngarathwsngayttyytnmgnta
ygarytnmgntayggnccnmgngayccnaaraaywsnacnggnccnacng
tnathcarytnathgcnacngaracntgytgyccngcnytncarmgnccn
caywsngcnwsngcnytngaycarwsnccntgygcncarccnacnatgcc
ntggcargayggnccnaarcaracnwsnccnwsnmgngargcnwsngcny
tnacngcngarggnggnwsntgyytnathwsnggnytncarccnggnaay
wsntaytggytncarytnmgnwsngarccngayggnathwsnytnggngg
nwsntggggnwsntggwsnytnccngtnacngtngayytnccnggngayg
cngtngcnytnggnytncartgyttyacnytngayytnaaraaygtnacn
tgycartggcarcarcargaycaygcnwsnwsncarggnttyttytayca
ywsnmgngcnmgntgytgyccnmgngaymgntayccnathtgggaraayt
gygargargargaraaracnaayccnggnytncaracnccncarttywsn
mgntgycayttyaarwsnmgnaaygaywsnathathcayathytngtnga
rgtnacnacngcnccnggnacngtncaywsntayytnggnwsnccnttyt
ggathcaycargcngtnmgnytnccnacnccnaayytncaytggmgngar
athwsnwsnggncayytngarytngartggcarcayccnwsnwsntgggc
ngcncargaracntgytaycarytnmgntayacnggngarggncaycarg
aytggaargtnytngarccnccnytnggngcnmgnggnggnacnytngar
ytnmgnccnmgnwsnmgntaymgnytncarytnmgngcnmgnytnaaygg
nccnacntaycarggnccntggwsnwsntggwsngayccnacnmgngtng
aracngcnacngaracngcntggathwsnytngtnacngcnytncayytn
gtnytnggnytnwsngcngtnytnggnytnytnytnytntaycayggnca
rmgncaywsngaygarcaycaycaygaygaywsnytnccncayccncarc
argcnacngaygaywsnggncaygarwsngaywsnaaywsnaaygarggn
mgncaycayytnytngtnwsnggngcnggngayggnccnccnytntgyws
ncaraayytnggngcnccnggnggnggnccngayaayggnccncargayc
cngayaayacngaygayaayggnccncargayccngayaayacngaygay
aayggnccncaygayccnytnccncargayccngayaayacngaygayaa
yggnccncargayccngayaayacngaygayaayggnccncaygayccny
tnccncaywsnccnwsngaywsngcnggnaaygayggnggnccnccncar
ytnacngargargtngaraayaarggnggngaycarggnccnccnytnat
gacngayggnggnggnggncaywsncaygaywsnggncayggnggnggng
ayccncayytnccnacnytnytnytnggnwsnwsnggnwsnggnggngay
gaygaygayccncayggnccngtncarytnwsntaytaygaytrrtrr

SEQ ID NO: 13: TpoRec.TpoRtm.CD137cyto

555 amino acids presented in the N- to C-terminus direction, of which 1-491 (bold): TpoR extracellular domain, 492-513 (bold, underlined): TpoR TM domain, 514-555 (unformatted): CD137 cytoplasmic domain.

MPSWALFMVTSCLLLAPQNLAQVSSQDVSLLASDSEPLKCFSRTFEDLTC
FWDEEEAAPSGTYQLLYAYPREKPRACPLSSQSMPHFGTRYVCQFPDQEE
VRLFFPLHLWVKNVFLNQTRTQRVLFVDSVGLPAPPSIIKAMGGSQPGEL
QISWEEPAPEISDFLRYELRYGPRDPKNSTGPTVIQLIATETCCPALQRP
HSASALDQSPCAQPTMPWQDGPKQTSPSREASALTAEGGSCLISGLQPGN
SYWLQLRSEPDGISLGGSWGSWSLPVTVDLPGDAVALGLQCFTLDLKNVT
CQWQQQDHASSQGFFYHSRARCCPRDRYPIWENCEEEEKTNPGLQTPQFS
RCHFKSRNDSIIHILVEVTTAPGTVHSYLGSPFWIHQAVRLPTPNLHWRE
ISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHL
VLGLSAVLGLLLLKRGRKKLLYIFKQPFMRPVQTTQEEDGCSCRFPEEEE
GGCEL**
SEQ ID NO: 27: TpoRec.TpoRtm.CD137cyto
atgccnwsntgggcnytnttyatggtnacnwsntgyytnytnytngcncc
ncaraayytngcncargtnwsnwsncargaygtnwsnytnytngcnwsng
aywsngarccnytnaartgyttywsnmgnacnttygargayytnacntgy
ttytgggaygargargargcngcnccnwsnggnacntaycarytnytnta
ygcntayccnmgngaraarccnmgngcntgyccnytnwsnwsncarwsna
tgccncayttyggnacnmgntaygtntgycarttyccngaycargargar
gtnmgnytnttyttyccnytncayytntgggtnaaraaygtnttyytnaa
ycaracnmgnacncarmgngtnytnttygtngaywsngtnggnytnccng
cnccnccnwsnathathaargcnatgggnggnwsncarccnggngarytn
carathwsntgggargarccngcnccngarathwsngayttyytnmgnta
ygarytnmgntayggnccnmgngayccnaaraaywsnacnggnccnacng
tnathcarytnathgcnacngaracntgytgyccngcnytncarmgnccn
caywsngcnwsngcnytngaycarwsnccntgygcncarccnacnatgcc
ntggcargayggnccnaarcaracnwsnccnwsnmgngargcnwsngcny
tnacngcngarggnggnwsntgyytnathwsnggnytncarccnggnaay
wsntaytggytncarytnmgnwsngarccngayggnathwsnytnggngg
nwsntggggnwsntggwsnytnccngtnacngtngayytnccnggngayg
cngtngcnytnggnytncartgyttyacnytngayytnaaraaygtnacn
tgycartggcarcarcargaycaygcnwsnwsncarggnttyttytayca
ywsnmgngcnmgntgytgyccnmgngaymgntayccnathtgggaraayt
gygargargargaraaracnaayccnggnytncaracnccncarttywsn
mgntgycayttyaarwsnmgnaaygaywsnathathcayathytngtnga
rgtnacnacngcnccnggnacngtncaywsntayytnggnwsnccnttyt
ggathcaycargcngtnmgnytnccnacnccnaayytncaytggmgngar
athwsnwsnggncayytngarytngartggcarcayccnwsnwsntgggc
ngcncargaracntgytaycarytnmgntayacnggngarggncaycarg
aytggaargtnytngarccnccnytnggngcnmgnggnggnacnytngar
ytnmgnccnmgnwsnmgntaymgnytncarytnmgngcnmgnytnaaygg
nccnacntaycarggnccntggwsnwsntggwsngayccnacnmgngtng
aracngcnacngaracngcntggathwsnytngtnacngcnytncayytn
gtnytnggnytnwsngcngtnytnggnytnytnytnytnaarmgnggnmg
naaraarytnytntayathttyaarcarccnttyatgingnccngtncar
acnacncargargargayggntgywsntgymgnttyccngargargarga
rggnggntgygarytntrrtrr

SEQ ID NO: 14: TpoRec.TpoRtm.CD28cyto

554 amino acids presented in the N- to C-terminus direction, of which 1-491 (bold): TpoR extracellular domain, 492-513 (bold, underlined): TpoR TM domain, 514-554 (unformatted): CD28 cytoplasmic domain.

MPSWALFMVTSCLLLAPQNLAQVSSQDVSLLASDSEPLKCFSRTFEDLTC
FWDEEEAAPSGTYQLLYAYPREKPRACPLSSQSMPHFGTRYVCQFPDQEE
VRLFFPLHLWVKNVFLNQTRTQRVLFVDSVGLPAPPSIIKAMGGSQPGEL
QISWEEPAPEISDFLRYELRYGPRDPKNSTGPTVIQLIATETCCPALQRP
HSASALDQSPCAQPTMPWQDGPKQTSPSREASALTAEGGSCLISGLQPGN
SYWLQLRSEPDGISLGGSWGSWSLPVTVDLPGDAVALGLQCFTLDLKNVT
CQWQQQDHASSQGFFYHSRARCCPRDRYPIWENCEEEEKTNPGLQTPQFS
RCHFKSRNDSIIHILVEVTTAPGTVHSYLGSPFWIHQAVRLPTPNLHWRE
ISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHL
VLGLSAVLGLLLLRSKRSRLLHSDYMNMTPRRPGPTRKHVQPVAPPRDFA
AVRS**
SEQ ID NO: 28: TpoRec.TpoRtm.CD28cyto
atgccnwsntgggcnytnttyatggtnacnwsntgyytnytnytngcncc
ncaraayytngcncargtnwsnwsncargaygtnwsnytnytngcnwsng
aywsngarccnytnaartgyttywsnmgnacnttygargayytnacntgy
ttytgggaygargargargcngcnccnwsnggnacntaycarytnytnta
ygcntayccnmgngaraarccnmgngcntgyccnytnwsnwsncarwsna
tgccncayttyggnacnmgntaygtntgycarttyccngaycargargar
gtnmgnytnttyttyccnytncayytntgggtnaaraaygtnttyytnaa
ycaracnmgnacncarmgngtnytnttygtngaywsngtnggnytnccng
cnccnccnwsnathathaargcnatgggnggnwsncarccnggngarytn
carathwsntgggargarccngcnccngarathwsngayttyytnmgnta
ygarytnmgntayggnccnmgngayccnaaraaywsnacnggnccnacng
tnathcarytnathgcnacngaracntgytgyccngcnytncarmgnccn
caywsngcnwsngcnytngaycarwsnccntgygcncarccnacnatgcc
ntggcargayggnccnaarcaracnwsnccnwsnmgngargcnwsngcny
tnacngcngarggnggnwsntgyytnathwsnggnytncarccnggnaay
wsntaytggytncarytnmgnwsngarccngayggnathwsnytnggngg
nwsntggggnwsntggwsnytnccngtnacngtngayytnccnggngayg
cngtngcnytnggnytncartgyttyacnytngayytnaaraaygtnacn
tgycartggcarcarcargaycaygcnwsnwsncarggnttyttytayca
ywsnmgngcnmgntgytgyccnmgngaymgntayccnathtgggaraayt
gygargargargaraaracnaayccnggnytncaracnccncarttywsn
mgntgycayttyaarwsnmgnaaygaywsnathathcayathytngtnga
rgtnacnacngcnccnggnacngtncaywsntayytnggnwsnccnttyt
ggathcaycargcngtnmgnytnccnacnccnaayytncaytggmgngar
athwsnwsnggncayytngarytngartggcarcayccnwsnwsntgggc
ngcncargaracntgytaycarytnmgntayacnggngarggncaycarg
aytggaargtnytngarccnccnytnggngcnmgnggnggnacnytngar
ytnmgnccnmgnwsnmgntaymgnytncarytnmgngcnmgnytnaaygg
nccnacntaycarggnccntggwsnwsntggwsngayccnacnmgngtng
aracngcnacngaracngcntggathwsnytngtnacngcnytncayytn
gtnytnggnytnwsngcngtnytnggnytnytnytnytnmgnwsnaarmg
nwsnmgnytnytncaywsngaytayatgaayatgacnccnmgnmgnccng
gnccnacnmgnaarcaytaycarccntaygcnccnccnmgngayttygcn
gcntaymgnwsntrrtrr

The invention is further described by the following numbered paragraphs:

1. A T or NK cell comprising a chimeric recombinant growth factor receptor (CrGFR) comprising:
(i) an extracellular (EC) domain;
(ii) a thrombopoietin transmembrane (TM) domain; and
(iii) a first intracellular (IC) domain; and, optionally,
(iv) a second intracellular domain.
2. The T or NK cell according to paragraph 1 wherein binding of a ligand to the CrGFR induces proliferation of the T or NK cell.
3. The T or NK cell according to paragraph 2 wherein the ligand is human thrombopoietin, a thrombopoietin receptor agonist, or a tumour associated antigen.
4. The T or NK cell according to paragraph 3 wherein the thrombopoietin receptor agonist binds to the TM domain.
5. The T or NK cell according to paragraph 3 or paragraph 4 wherein the thrombopoietin receptor agonist is selected from Eltrombopag and Romiplostim.
6. The T or NK cell according to the preceding paragraphs wherein the EC domain comprises the human c-mpl (thrombopoietin) EC domain.
7. The T or NK cell according to paragraphs 1 to 5 wherein the EC domain comprises one or more of i) a truncated EC domain, ii) a truncated c-mpl EC domain, iii) a domain that binds to a tumour associated antigen, iv) an antibody or antibody fragment that binds to a tumour associated antigen; and v) a selection marker.
8. The T or NK cell according to the preceding paragraphs wherein the first IC domain is selected from human growth hormone receptor, human prolactin receptor, human thrombopoietin receptor (c-mpl), G-CSF receptor, GM-CSF receptor, LMP, IL2, CD28 or CD137.
9. The T or NK cell according to the preceding paragraphs wherein the first IC domain comprises the IC domain from human thrombopoietin receptor (c-mpl), or a truncated IC domain from human thrombopoietin receptor (c-mpl).
10. The T or NK cell according to the preceding paragraphs wherein the second IC domain is from human growth hormone receptor, human prolactin receptor, human thrombopoietin receptor (c-mpl), G-CSF receptor or GM-CSF receptor, a costimulatory receptor, a cytokine receptor or a cosignalling receptor.
11. The T or NK cell according to paragraph 8 or paragraph 9 wherein the second IC domain is selected from human thrombopoietin receptor (c-mpl), or a truncated IC domain from human thrombopoietin receptor (c-mpl) preferably TpoR 460, CD40, IL2rβ, IL2Rγ, ITAM1 or LMP1.
12. The T or NK cell according to the preceding paragraphs wherein the CrGFR comprises the TM sequence shown in SEQ ID NO: 1, or a variant thereof having at least 80% sequence identity, which binds human thrombopoietin or a thrombopoietin receptor agonist.
13. The T or NK cell comprising a chimeric recombinant growth factor receptor (CrGFR), wherein the CrGFR comprises the sequence shown as SEQ ID NOS: 3, 4, 5, 6, 7, 8, 9, 10, 11, 12, 13, or 14, or a variant thereof having at least 80%, 85%, 90% 95% 97% or 99% sequence identity which binds human thrombopoietin or a thrombopoietin receptor agonist.
14. A T or NK cell according to paragraph 13 wherein binding by thrombopoietin, or a human thrombopoietin receptor agonist induces cell proliferation and/or survival.
15. The T cell or NK cell according to any preceding paragraph which binds to Eltrombopag.
16. The T cell or NK cell according to any preceding paragraph wherein the T cell is selected from a Tumour Infiltrating Lymphocyte (TIL) a T Regulatory Cell (Treg) or a primary T cell.
17. The T cell or NK cell according to any preceding paragraph further comprising a recombinant T-cell receptor (TCR) and/or Chimeric Antigen Receptor (CAR).
18. A chimeric recombinant growth factor receptor (CrGFR) as defined in any preceding paragraph.
19. A cell comprising the chimeric recombinant growth factor receptor (CrGFR) according to paragraph 18.
20. A nucleic acid sequence encoding the CrGFR as defined in any preceding paragraph.
21. A nucleic acid sequence according to paragraph 20 which comprises the sequence shown as SEQ ID NOS: 15, 16, 17, 18, 19, 20, 21, 22, 23, 24, 25, 26, 27 or 28.
22. A vector which comprises a nucleic acid sequence according to paragraph 20 or 21.
23. A method for making a T cell or NK cell according to any of paragraphs 1-17, which comprises the step of introducing a nucleic acid according to paragraph 20 or 21, or vector according to paragraph 22, into a T cell or NK cell.
24. A pharmaceutical composition which comprises a vector according to paragraph 22 or a T or NK cell according to paragraphs 1-17, together with a pharmaceutically acceptable carrier, diluent or excipient.
25. A method of in-vivo cell expansion comprising administering the cells of paragraphs 1-17, or pharmaceutical composition of paragraph 24 to a subject.
26. A method of in-vivo cell expansion according to paragraph 25 comprising administering thrombopoietin, or a thrombopoietin receptor agonist such as Eltrombopag or Romiplostim, to a subject.
27. A T or NK cell according to any of paragraphs 1-17, or vector according to paragraph 22, for use in adoptive cell therapy.
28. A T or NK cell according to any of paragraphs 1-17, or vector according to paragraph 22, for use in a method of treating cancer.
29. A method for treating cancer which comprises the step of administering the T cell or NK cell according to any of paragraphs 1-17 to a subject.
30. The use of a vector according to paragraph 22 or the T or NK cell according to any of paragraphs 1-17 in the manufacture of a medicament for treating cancer.
31. Eltrombopag for use in the in-vitro or in-vivo expansion of T or NK cells according to any of paragraphs 1-17.
32. A composition comprising a T or NK cell according to paragraphs 1 to 17 for use in combination with thrombopoietin or a thrombopoietin receptor agonist in the treatment of a cancer.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A T or NK cell comprising a chimeric recombinant growth factor receptor (CrGFR) comprising:
(i) an extracellular (EC) domain;
(ii) a thrombopoietin transmembrane (TM) domain; and
(iii) a first intracellular (IC) domain; and, optionally,
(iv) a second intracellular domain.

2. The T or NK cell according to claim 1 wherein binding of a ligand to the CrGFR induces proliferation of the T or NK cell.

3. The T or NK cell according to claim 2 wherein the ligand is human thrombopoietin, a thrombopoietin receptor agonist, or a tumour associated antigen.

4. The T or NK cell according to claim 3 wherein the thrombopoietin receptor agonist binds to the TM domain.

5. The T or NK cell according to claim 3 wherein the thrombopoietin receptor agonist is Eltrombopag or Romiplostim.

6. The T or NK cell according claim 1 wherein the EC domain comprises the human c-mpl (thrombopoietin) EC domain.

7. The T or NK cell according to claim 1 wherein the EC domain comprises one or more of i) a truncated EC domain, ii) a truncated c-mpl EC domain, iii) a domain that binds to a tumour associated antigen, iv) an antibody or antibody fragment that binds to a tumour associated antigen; and v) a selection marker.

8. The T or NK cell according to claim 1 wherein the first IC domain is selected from human growth hormone receptor, human prolactin receptor, human thrombopoietin receptor (c-mpl), G-CSF receptor, GM-CSF receptor, LMP, IL2, CD28 or CD137.

9. The T or NK cell according to claim 1 wherein the first IC domain comprises the IC domain from human thrombopoietin receptor (c-mpl), or a truncated IC domain from human thrombopoietin receptor (c-mpl).

10. The T or NK cell according to claim 1 wherein the second IC domain is from human growth hormone receptor, human prolactin receptor, human thrombopoietin receptor (c-mpl), G-CSF receptor or GM-CSF receptor, a costimulatory receptor, a cytokine receptor or a cosignalling receptor.

11. The T or NK cell according to claim 8 or claim 9 wherein the second IC domain is selected from human thrombopoietin receptor (c-mpl), or a truncated IC domain from human thrombopoietin receptor (c-mpl) preferably TpoR Δ60, CD40, IL2rβ, IL2Rγ, ITAM1 or LMP1.

12. The T or NK cell according to claim 1 wherein the CrGFR comprises the TM sequence shown in SEQ ID NO: 1, or a variant thereof having at least 80% sequence identity, which binds human thrombopoietin or a thrombopoietin receptor agonist.

13. The T or NK cell comprising a chimeric recombinant growth factor receptor (CrGFR), wherein the CrGFR comprises the sequence shown as SEQ ID NOS: 3, 4, 5, 6, 7, 8, 9, 10, 11, 12, 13, or 14, or a variant thereof having at least 80%, 85%, 90% 95% 97% or 99% sequence identity which binds human thrombopoietin or a thrombopoietin receptor agonist.

14. A T or NK cell according to claim 13 wherein binding by thrombopoietin, or a human thrombopoietin receptor agonist induces cell proliferation and/or survival.

15. The T cell or NK cell according to claim 1 which binds to Eltrombopag.

16. The T cell or NK cell according to claim 1 wherein the T cell is selected from a Tumour Infiltrating Lymphocyte (TIL) a T Regulatory Cell (Treg) or a primary T cell.

17. The T cell or NK cell according to claim 1 further comprising a recombinant T-cell receptor (TCR) and/or Chimeric Antigen Receptor (CAR).

18. A chimeric recombinant growth factor receptor (CrGFR) as defined in claim 1.

19. A cell comprising the chimeric recombinant growth factor receptor (CrGFR) according to claim 18.

20. A nucleic acid sequence encoding the CrGFR as defined in claim 1.

21. A nucleic acid sequence according to claim 20 which comprises the sequence shown as SEQ ID NOS: 15, 16, 17, 18, 19, 20, 21, 22, 23, 24, 25, 26, 27 or 28.

22. A vector which comprises a nucleic acid sequence according to claim 20.

23. A method for making a T cell or NK cell according to claim 1, which comprises the step of introducing a nucleic acid according to claim 20 into a T cell or NK cell.

24. A pharmaceutical composition which comprises a vector according to claim 22 or a T or NK cell according to claim 1, together with a pharmaceutically acceptable carrier, diluent or excipient.

25. A method of in-vivo cell expansion comprising administering the cells of claim 1, or pharmaceutical composition of claim 24 to a subject.

26. A method of in-vivo cell expansion according to claim 25 comprising administering thrombopoietin, or a thrombopoietin receptor agonist such as Eltrombopag or Romiplostim, to a subject.

27. A T or NK cell according to claim 1, or vector according to claim 22, for use in adoptive cell therapy.

28. A T or NK cell according to claim 1, or vector according to claim 22, for use in a method of treating cancer.

29. A method for treating cancer which comprises the step of administering the T cell or NK cell according to claim 1 to a subject.
